(19) United States
(12) Patent Application Publication
(10) Pub. No.: US 2024/0307419 A1
(43) Pub. Date: Sep. 19, 2024

(54) CONFORMATIONALLY-CONSTRAINED INHIBITORS OF 3C OR 3C-LIKE PROTEASES

(71) Applicants: Kansas State University Research Foundation, Manhattan, KS (US); Wichita State University, Wichita, KS (US); University of Iowa Research Foundation, Iowa City, IA (US)

(72) Inventors: Kyeong-Ok CHANG, Manhattan, KS (US); Yunjeong KIM, Manhattan, KS (US); William C. GROUTAS, Wichita, KS (US); Stanley PERLMAN, Iowa City, IA (US)

(21) Appl. No.: 18/262,970

(22) PCT Filed: Jan. 28, 2022
(86) PCT No.: PCT/US2022/014375

§ 371 Date: Jul. 26, 2023

Related U.S. Application Data
(60) Provisional application No. 63/143,627, filed on Jan. 29, 2021.

(51) Int. Cl. A61K 31/675; A61K 31/4025; A61K 31/438; A61P 31/12; C07D 207/267; C07D 403/12; C07F 9/6584

(52) U.S. Cl. CPC A61K 31/675; A61K 31/4025; A61K 31/438; A61P 31/12; C07D 207/267; C07D 403/12; C07F 9/65844

(57) ABSTRACT

Compounds and treatment methods with compounds exhibiting antiviral activity and/or inhibition of viral replication against viruses, particularly those belonging to the picornavirus-like supercluster, including coronavirus.

### CROSS-REFERENCE TO RELATED APPLICATIONS

The present application claims the priority benefit of U.S. Provisional Patent Application Ser. No. 63/143,627, filed Jan. 29, 2021, entitled CONFORMATIONALLY-CONSTRAINED INHIBITORS OF 3C OR 3C-LIKE PROTEASES, incorporated by reference in its entirety herein.

### STATEMENT REGARDING FEDERALLY-FUNDED RESEARCH

This invention was made with U.S. Government support under grant numbers R01 AI109039 and R01 161085 awarded by the National Institutes of Health. The government has certain rights in the invention.

### BACKGROUND

### Field

The present disclosure relates to broad-spectrum antiviral compounds targeting the 3C-like proteases of coronavirus.

### Description of Related Art

Many viruses encode polyproteins with proteases which catalyze their subsequent cleavage to the mature functional proteins and are essential for viral replication. Previous attempts have been made to inhibit viral activity by targeting such proteases. However, most protease inhibitors have a short range of specificity that is genus-, species-, or even strain-specific due to structural variations in the viral proteases. Thus, broad spectrum antivirals are rare and have proven elusive to researchers.

Highly pathogenic coronaviruses are a significant threat to public health, as exemplified by Severe Acute Respiratory Syndrome coronavirus (SARS-CoV), Middle East respiratory syndrome coronavirus (MERS-CoV), and more recently emerged SARS-CoV-2, the causative agent of coronavirus disease 2019 (COVID19). Other members of the picornavirus-like supercluster, such as caliciviruses (including norovirus and sapovirus genera) and picornaviruses share a common feature with coronaviruses in that they also possess a viral 3C or 3CL protease which is responsible for most cleavages of the corresponding viral polyprotein. These 3C and 3CL proteases share some common characteristics, including atypical chymotrypsin-like fold and a catalytic triad (or dyad) with Cys-His-Glu (or Asp) on the protease, and a preference for a Glu or Gln residue at the P1 position on the substrate. Caliciviruses include noroviruses (Norwalk virus [NV]), feline calicivirus, MD145, murine norovirus [MNV], vesicular exanthema of swine virus, and rabbit hemorrhagic disease virus. Picornaviruses include enteroviruses (such as enterovirus 71), poliovirus, coxsackievirus, foot-and-mouth disease virus (FMDV), hepatitis A virus (HAV), porcine teschovirus, and rhinovirus (cause of common cold).

Coronaviruses, in particular, are a large group of viruses that can cause a wide variety of diseases in humans and animals. Coronaviruses include human coronavirus (cause of the common cold such as 229E strain), transmissible gastroenteritis virus (TGEV), murine hepatitis virus (MHV), bovine coronavirus (BCV), feline infectious peritonitis virus (FIPV), and the above-mentioned MERS and SARS viruses. Most human coronaviruses generally cause the common cold, a mild upper respiratory illness. However, global outbreaks of new human coronavirus infections with severe respiratory disease have periodically emerged from animals, which includes SARS-CoV, MERS-CoV, and most recently, SARS-CoV-2, which emerged in China in December 2019 and quickly spread throughout the world. The genetic analysis of SARS-CoV-2 showed it to be closely related to SARS-like beta-coronaviruses of bat origin, bat-SL-CoVZC45 and bat-SL-CoVZXC21. Despite the periodic emergence of novel coronaviruses infecting humans, there are no broadly effective FDA-approved vaccines or antiviral drugs against these viruses, underscoring an urgent need for the development of preventive and therapeutic measures against coronaviruses.

The SARS-CoV-2 genome is large (˜30 kb) and similar to the genomes of SARS-CoV and MERS-CoV (˜80% and ˜50% sequence identity, respectively). It contains two open reading frames (ORF1a and ORF1b) and encodes multiple structural and nonstructural proteins. Translation of the genomic mRNA of ORF1a yields a polyprotein (pp1a), while a second polyprotein (pp1b) is the product of a ribosomal frameshift that joins ORF1a together with ORF1b. The two polyproteins are processed by a 3C-like protease (3CLpro, also referred to as Main protease, Mpro) (11 cleavage sites) and a papain-like cysteine protease (PLpro), resulting in 16 mature nonstructural proteins including an RNA-dependent RNA polymerase (RdRp) which are involved in the replication-transcription complex. Both 3CLpro and PLpro are essential for viral replication, making them attractive targets for drug development. Coronavirus 3CLpro is a chymotrypsin-like cysteine protease that has two N-terminal domains containing two β-barrel chymotrypsin-like folds. The active site of 3CLpro is located in the cleft between the two domains and is characterized by a catalytic Cys148-His41 dyad.

Two years since its emergences, the COVID-19 pandemic remains a major concern for public health worldwide and there is an urgent need for the creation of effective therapeutics, including vaccines, biologics, and small molecule therapeutics, to combat SARS-CoV-2, and emerging variants. Inspection of the virus life cycle reveals multiple viral and host-based choke points that can be exploited to combat the virus. SARS-CoV-2 3CL protease, an enzyme essential for viral replication, is an attractive viral choke point and the design of inhibitors of the protease may lead to the development of effective SARS-CoV-2-specific antivirals.

### SUMMARY

Our foray in this area has resulted in the development of broad-spectrum inhibitors of an array of viruses, including coronaviruses and noroviruses that encode 3CLpro as well as the first demonstration of clinical efficacy of a coronavirus 3CLpro inhibitor (GC376, currently in clinical development, see U.S. Pat. No. 9,474,759, issued Oct. 25, 2016, incorporated by reference herein in its entirety). Specifically, administration of a 3CLpro inhibitor to cats with feline infectious peritonitis (FIP), a coronavirus-induced systemic disease that is 100% fatal, reversed the progression of FIP and resulted in clinical remission. Here, we report a series of non-deuterated and deuterated 3CLpro inhibitors that are highly potent against multiple coronaviruses including MERS-CoV, SARS-CoV, and SARS-CoV-2.

These novel compounds incorporate new design elements (designated as X in structure (I)) into the inhibitor backbone which leverage spatial and/or 3-dimensional geometric orientation with their target(s) (e.g., the S4 pocket in the active site of 3CLpro), thus allowing the compound moieties to be projected in multiple vectors at the target site and enhance binding. These design elements include bicyclic and tricyclic cycloalkane derivatives, particularly bridge bi- or tricycles (preferably cyclohexanes), as well as nitrogen, oxygen or sulfur-containing heterocycles, including bi- or tricycles, such as pyrrolidine derivatives, piperidine derivatives, spirocycles, and as well as phosphorous-containing rings such as phospholane derivatives, and the like. Some embodiments include conformationally-constrained moieties to reduce isomerization or lock in cis or trans conformations and reduce the conformational variability of the compounds envisaged to exploit new chemical space and to optimally engage in favorable binding interactions with the active site of the target protease. Furthermore, several deuterated variants are also synthesized to potentially improve the PK properties and ancillary parameters of the inhibitor.

The basic backbone (I) acts as a starting point for synthesizing numerous compounds in this series, as follows:

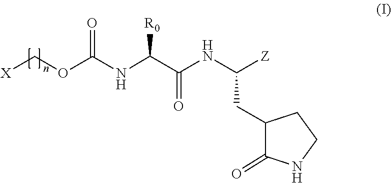

In the foregoing structures, each R₀ is a branched or unbranched alkyl, fluorine-containing branched or unbranched alkyl, cycloalkyl, aryl, arylalkyl, alkenyl, alkynyl, natural or unnatural amino acid side chain, or a combination thereof, and in particular leucine (Leu), cyclohexylalanine (Cha), or a fluorinated side chain. In combination with the glutamine surrogate, the side chain forms part of the recognition element of the inhibitor with substrate specificity for the target protease subsite. The selectivity of the inhibitor for the targeted enzyme is embodied in the structure of the recognition element (glutamine surrogate fragment with side chain).

In the foregoing structures, each X is part of the peptidyl design element in the structure responsible for correct positioning of the inhibitor relative to the active site of the target enzyme, resulting in the reversible formation of the initial enzyme:inhibitor complex. The X moiety can be directly connected to the backbone (i.e., where n is 0) or via a branched or unbranched C₁-C₆ alkyl linkage (i.e., where n 1-6) (or deuterated variant thereof). In one or more embodiments, X is selected from the group consisting of polycyclic cycloalkanes, particularly polycyclic cyclohexane derivatives, preferably bridged polycycles (bi- and tri-), as well as nitrogen, oxygen, phosphorous, or sulfur-containing heterocycles and polycycles, particularly 5-member heterocycles, such as piperidine derivatives, pyrrolidine derivatives, pyrrolidinones, pyrrolidine, phospholane derivatives, as well as azetidines, and spirocycles, as well as halogenated derivatives thereof (preferably fluorinated). In one or more embodiments, X is a polycyclic cycloalkane, bridged polycycle, nitrogen-containing heterocycle or polycycle, azetidine, or spirocycle. In one or more embodiments, X is preferably a nitrogen-containing heterocycle or polycycle. In one or more embodiments, the nitrogen-containing heterocycle comprises at least one ring nitrogen atom and 0-3 additional ring heteroatoms selected from the group consisting of nitrogen, oxygen, phosphorous, and sulfur. In most embodiments, X preferably comprises saturated moieties, although in some embodiments, one or more of the cyclic moieties may include an unsaturated (double) bond. In some embodiments, saturated cyclic moieties may be substituted with one or more aromatic moieties, preferably phenyl or benzyl groups, directly or via a C₁-C₃ alkyl linkage.

In the foregoing, structures, Z is the warhead, which denotes the moiety in the inhibitor structure that reacts with the active site cysteine resulting in inactivation of the enzyme. Each Z is selected from the group consisting of C₁-C₆ hydroxyalkyl, aldehydes, alpha-ketoamides, alpha-ketoheterocycles, and bisulfite salts (aldehyde bisulfite adducts), as well as the bisulfite adducts of alpha-ketoamides and alpha-ketocycles. In particular, Z can be —CH₂OH, —CHO, —CH(OH)SO₃⁻Na⁺, —[O(C═O)R_{z}]SO₃⁻Na⁺, and —(C═O)heterocycle, where R_{z} is an alkyl or arylalkyl with —CH₃ and —CH₂CH₃ being preferred, and the heterocycle is a benzothiazole, oxadiazole, and the like.

The Examples exemplify a series of non-deuterated and deuterated dipeptidyl aldehyde inhibitors that incorporate in their structure a conformationally-constrained polycyclohexane moieties that were synthesized and found to potently inhibit SARS-CoV-2 3CL protease in biochemical and cell-based assays, as well as inhibitors containing pyrrolidine derivatives, azetidines, and spirocycles. The corresponding latent aldehyde bisulfite adducts are also examined found to be equipotent to the precursor aldehydes. High-resolution cocrystal structures confirmed the mechanism of action and illuminated the structural determinants involved in binding.

The spatial disposition of the compounds disclosed herein provides an effective means of accessing new chemical space and optimizing pharmacological activity. Furthermore, the lack of cytotoxicity and cellular permeability of the identified inhibitors warrants their advancement as potential therapeutics for COVID-19.

Thus, embodiments described herein include methods of treating or preventing viral infection in a subject from one or more coronaviruses as well as against other viruses that belong to the picornavirus-like supercluster, including caliciviruses and picornaviruses is also provided. The method comprises administering to said subject a therapeutically-effective amount of a first antiviral compound according to the various embodiments described herein.

A broad spectrum antiviral composition is also disclosed. The composition comprises a first antiviral compound according to the various embodiments described herein dispersed in a pharmaceutically-acceptable carrier.

A kit is also provided herein. The kit comprises: an antiviral compound according to the various embodiments described herein; and instructions for administering the compound to a subject in need thereof.

A method of preventing or inhibiting replication of a virus in a cell is also disclosed. The method comprises contacting a coronavirus, picornavirus, or calicivirus cell with a compound according to the various embodiments described herein.

The disclosure is also concerned with the use of a compound according to the various embodiments described herein to prepare a therapeutic or prophylactic medicament for the treatment or prevention of a viral infection from coronaviruses, picornavirus, or calicivirus in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows reaction Scheme 1 for the stepwise compound synthesis with intermediate compounds for 3C-like protease (3CLpro) inhibitors 2a-2o and 3a-3o.

FIG. 2 shows X-ray crystal structures for the binding mode of 2a (gray) with SARS-CoV-2 3CLpro associated with subunit A (A, C) and subunit B (B, D). Fo-Fc Polder omit map (green mesh) contoured at 3a (A, B). Hydrogen bond interactions (dashed lines) (C, D). Surface representations showing the orientation of 2a in subunit A (E) and subunit B (F) near the S4 subsite of SARS2 3CLpro with neighboring residues colored yellow (nonpolar), cyan (polar), and white (weakly polar).

FIG. 3 shows X-ray crystal structures for the binding mode of 3c (A, B, E) and its deuterated analog 3d (C, D, F) with SARS-CoV-2 3CLpro. Fo-Fc Polder omit map (green mesh) contoured at 36 (A, B). Hydrogen bond interactions (dashed lines) (C, D). E/F: Surface representation showing the orientation of 3c (E) and 3d (F) near the S4 subsite of SARS-CoV-2 3CLpro with neighboring residues colored yellow (nonpolar), cyan (polar), and white (weakly polar).

FIG. 4 shows X-ray crystal structures for the binding mode of 2f (A, B), 2k (C, D) and 3e (E, F) with SARS2-CoV-2 3CLpro. Fo-Fc Polder omit map (green mesh) contoured at 3a (A, C, E). Hydrogen bond interactions (dashed lines) (B, D, F).

FIG. 5 shows X-ray crystal structures showing superposition of all seven inhibitor bound structures 2a (red), 3b (blue), 2f (cyan), 2k (yellow), 3c (coral), 3d (magenta) and 3e (green). The bicyclic rings cover a space of approximately 6.3 Å in the S4 subsite.

FIG. 6 shows X-ray crystal structures for the binding mode of the deuterated analog 3b (gray) with SARS-CoV-2 3CLpro associated with subunit A (A, C) and subunit B (B, D). Fo-Fc Polder omit map (green mesh) contoured at 3a (A, B). Hydrogen bond interactions (dashed lines) (C, D). Superposition of 3b (gold) with 2a (gray) associated with subunit A (E) and subunit B (F).

FIG. 7 shows a comparison of inhibitors bound to SARS-CoV-2 3CLpro. Superposition of 3c (gray) and its deuterated analog 3d (gold).

FIG. 8 is a surface representation showing the orientation 2f (A), 2k (B) and 3e (C) subunit B near the S4 subsite of SARS-CoV-2 3CLpro with neighboring residues colored yellow (nonpolar), cyan (polar), and white (weakly polar).

FIG. 9 shows inhibition curves of selected compounds, 7b, 7c, 14b and 14c in the cell-based SARS-CoV-2 replicon assay.

FIG. 10 shows binding mode of the azetidine-derived inhibitor 14c to MERS-CoV 3CL^{pro} (A and C) and SARS-CoV-2 3CL^{pro} (B and D). Fo-Fc omit map (green mesh) contoured at 3σ (A and B). Hydrogen bond interactions (dashed lined) (C and D). PDB IDs: 14c with MERS-CoV 3CL^{pro} (7T41), 14c with SARS-CoV-2 3CLpro (7T4B).

FIG. 11 shows comparison of 15c bound to MERS-CoV 3CLpro and SARS-CoV-2 3CLpro. Surface representation showing the orientation of the inhibitor in MERS-CoV 3CLpro (A) and SARS-CoV-2 3CLpro (B) active site. Neighboring residues are colored yellow (nonpolar), cyan (polar), and white (weakly polar). Comparison of 15c binding mode in the MERS-CoV 3CLpro (gray) superimposed with SARS-CoV-2 3CLpro (gold) (C). PDB IDs: 15c bound to MERS-CoV 3CLpro (7T41), 15c SARS-CoV-2 3CLpro (7T4B).

FIG. 12 shows binding modes of 2-azaspiro [3.3] inhibitors 2c (A and D), 3c (B and E) and 4c (C and F) with SARS-CoV-2 3CL^{pro}. Fo-Fc omit map (green mesh) contoured at 3a (A-C). Hydrogen bond interactions (dashed lined) (D-F). PDB IDs: 2c (7T42), 3c (7T43), 4c (7T44).

FIG. 13 shows comparison of azaspiro [3.3] inhibitors bound to SARS-CoV-2 3CLpro. Surface representation showing the orientation of inhibitors 2c (A), 3c (B) and 4c (C) in the SARS-CoV-2 3CLpro active site. Neighboring residues are colored yellow (nonpolar), cyan (polar), and white (weakly polar). PDB IDs: 2c (7T42), 3c (7T43), 4c (7T44).

FIG. 14 shows superposition of 2c (blue), 3c (gold) and 4c (green) inhibitors bound to SARS-CoV-2 3CL^{pro} highlighting the broad conformations in the spirocyclic regions. PDB IDs: 2c (7T42), 3c (7T43), 4c (7T44).

FIG. 15 shows binding modes of 6-azaspiro [3.5] inhibitors 8c (A and D), 9c (B and E) and 10c (C and F) with SARS-CoV-2 3CL^{pro}. Fo-Fc omit map (green mesh) contoured at 3a (A-C). Hydrogen bond interactions (dashed lined) (D-F). PDB IDs: 8c (7T46), 9c (7T48), 10c (7T49).

FIG. 16 shows binding modes of azaspiro [3.5] inhibitors 7c (A and C) and 11c (B and D) with SARS-CoV-2 3CLpro. Fo-Fc omit map (green mesh) contoured at 3a (A and B). Hydrogen bond interactions (dashed lined) (C and D). PDB IDs: 7c (7T45), 11c (7T4A).

FIG. 17 shows comparison of 6-azaspiro [3.5] inhibitors complexed with SARS-CoV-2 3CL^{pro}. Superposition of 9c (coral) and 10c (gray) in complex with SARS-CoV-2 3CL^{pro}. The protein residues are colored gold and magenta for 9c and 10c respectively (A). Superposition of 7c (green), 8c (cyan), 9c (coral), 10c (gray) and 11c (pink) (B).

FIG. 18 shows comparison of azaspiro [3.5] inhibitors bound to SARS-CoV-2 3CLpro. Surface representation showing the orientation of inhibitor 7c (A), 8c (B), 11c (C), 10c (D) and 9c (E) with SARS-CoV-2 3CLpro. Neighboring residues are colored yellow (nonpolar), cyan (polar), and white (weakly polar). PDB IDs: 7c (7T45), 8c (7T46), 11c (7T4A), 10c (7T49) and 9c (7T48).

FIG. 19 shows binding modes of 6-azaspiro [3.5] inhibitors 8c (A and D), 9c (B and E) and 10c (C and F) with MERS-CoV 3CL^{pro}. Fo-Fc omit map (green mesh) contoured at 3σ (A-C). Hydrogen bond interactions (dashed lined) (D-F). PDB IDs: 8c (7T3Y), 9c (7T3Z), 10c (7T40).

FIG. 20 shows comparison of azaspiro [3.5] inhibitors 8c (A), 9c (B) and 10c (C) with MERS-CoV-2 3CLpro showing surface representations in the active site. Neighboring residues are colored yellow (nonpolar), cyan (polar), and white (weakly polar). PDB IDs: 8c (7T3Y), 9c (7T3Z) and 10c (7T40).

FIG. 21 shows superimposed MERS-CoV 3CLpro inhibitor bound structures 8c (coral), 9c (blue) and 10c (green). PDB IDs: 8c (7T3Y), 9c (7T3Z) and 10c (7T40).

### DETAILED DESCRIPTION

A series of novel protease inhibitors has been synthesized and shown to possess broad-spectrum activity against multiple coronaviruses including MERS-CoV, SARS-CoV, and SARS-CoV-2, as well as against other viruses that belong to the picornavirus-like supercluster, including caliciviruses and picornaviruses, in enzymatic and cell-based assays. The efficacy of the compounds in an animal model of MERS-CoV infection is also demonstrated. Members of this series of compounds are highly effective as antiviral therapeutics targeting a specific virus or, more importantly, they are broad-spectrum antivirals targeting multiple viruses. The wide applicability of the latter constitutes a significant advance in antiviral research and public health.

Embodiments described herein include antiviral compounds having broad-spectrum (multivalent) activity against coronaviruses as well as against other viruses that belong to the picornavirus-like supercluster, including caliciviruses and picornaviruses. The compounds are small-molecule based antivirals that effectively target and inhibit viral 3CL protease activity across multiple virus species, strains, and subtypes, thereby preventing formation of the mature virus and inhibiting virus replication in the host cell. In some embodiments, the compounds are prodrugs that are converted into active compounds that target and inhibit viral 3CL protease activity.

In some embodiments, antiviral compounds comprising (consisting essentially or even consisting of) formula (I), the series derivatives B, D, E, and F, described herein, or the pharmaceutically-acceptable salts thereof, are provided. In such compounds, the design element (X group) is selected from the group consisting of polycyclic cyclohexane derivatives, preferably bridged polycycles (bi- and tri-), as well as nitrogen, oxygen, phosphorous, or sulfur-containing heterocycles and polycycles, particularly 5-member heterocycles, such as piperidine derivatives, pyrrolidine derivatives, pyrrolidinones, fluorinated pyrrolidine, phospholane derivatives, as well as 4-member azetidines, and 4-, 5-, and 6-member spirocyclic compounds.

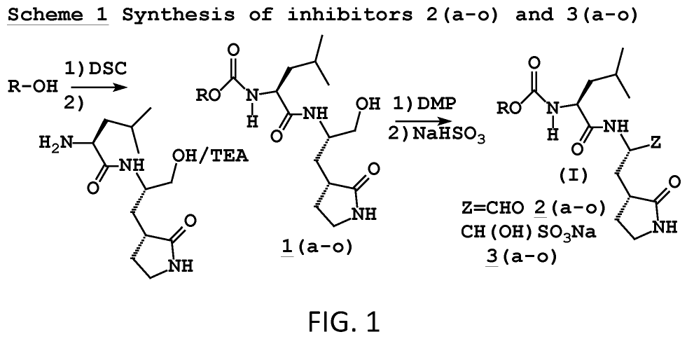

The recognition element in the inhibitor compounds encompasses the R₀ sidechain (preferably leucine) and glutamine surrogate fragment which drive substrate specificity to the viral protease, as well as the peptidyl design element (X group), which is configured to control spatial orientation of the compound for enhanced binding as well as other pharmacokinetic features of the inhibitor compounds. The design element X can be derived from one of following precursor compounds having a reactive primary or secondary alcohol group for conjugation with the glutamine surrogate and side chain fragment for synthesis of the inhibitors. In an improved reaction scheme, as illustrated in FIG. 1, the desired compound with a reactive primary or secondary alcohol group is first activated with N,N′-disuccinimidyl carbonate (DSC) to yield a mixed carbonate, followed by coupling with an amino alcohol to yield an intermediate alcohol product. This intermediate alcohol product can then be oxidized with Dess-Martin periodinane (DMP) to generate the desired aldehyde on the inhibitor. If desired, the aldehyde can be subsequently converted to the corresponding bisulfite adducts, or other warhead (Z) groups disclosed herein. Table 1 lists exemplary alcohol inputs for potential design moieties, as well as possible substituents therefor.

TABLE 1
X Design Moieties-Alcohol Inputs
Conformationally constrained cyclohexane derivatives (Series B)

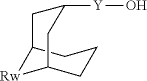

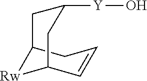

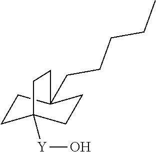

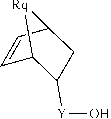

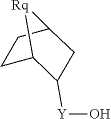

where each Y is (CRᵢRⱼ)ₙ, where n can be 0 (meaning Y is not present and the oxygen is directly
bonded to carbon of the ring) or 1-6 (meaning Y = CRᵢRⱼ where R₁ and Rⱼ can both be H or D, or
both methyl, or one H or D and the other methyl, each RW is independently selected from the
group consisting of —CH₂, —CF₂, and each Rq is independently —CH₂, —CH₂OH, —CH₂F, —COOH,
—(C═O)NHR′, or —(C═O)NHR′R′, or —CH(OH)R′, —CH₂OCH₂R′, where R′ is alkyl, phenyl,
substituted phenyl, or arylalkyl.

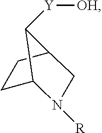

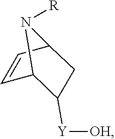

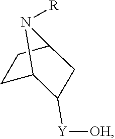

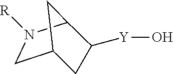

where each Y is (CRᵢRⱼ)ₙ, where n can be 0 (meaning Y is not present and the oxygen is directly
bonded to carbon of the ring) or 1-6 (meaning Y = CRᵢRⱼ where R₁ and Rⱼ can both be H or D, or
both methyl, or one H or D and the other methyl, and each R is H, alkyl, phenyl, substituted
phenyl, COOR′, SO₂R′, COR′, CONHR′, where R′ is an alkyl, aryl (phenyl), or arylalkyl.

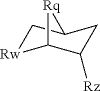

where Rq is —(CH₂)ₙ (where n is 1-2), —CHOH, —CH(OH)R′, —CH2OR′, —CHO(C═O)R′, —C═O,
—O, —OC═O, —CF₂, (C═O)R″, (C═O)NHR″, SO2R″, (C═O)OR″, NSO₂R″, NCOR′, NCONHR′,
and NR″, and where Rw is —CH2, —CF₂, —C═O, —CH(OH), _CH(OR″), NSO₂R′, NCOR′,
NCONHR′, and NR″, where R′ is alkyl, phenyl, substituted phenyl, or arylalkyl, and R″ is
alkyl, COOR′, phenyl, substituted phenyl or arylalkyl, and Rz is —H, —OH, —CH₂OH, or —CD₂OH.

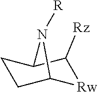

where R is H, alkyl, phenyl, substituted phenyl, COOR′, SO₂R′, COR′, CONHR′, where R′ is
an alkyl, aryl (phenyl), or arylalkyl, where Rw is —CH2, —CF₂, —C═O, —CH(OH), —CH(OR′),
NSO₂R′, NCOR′, NCONHR′, and NR″, where R′ is alkyl, phenyl, substituted phenyl, or
arylalkyl, and R″ is alkyl, COOR′, phenyl, substituted phenyl or arylalkyl, and Rz is —OH, —H,
—CH₂OH, or —CD₂OH.

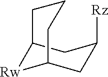

wherein Rz is —OH, —CH₂OH, —CD₂OH, —COOH, —COOR′, —CONHR′, —CONR′R′, wherein R′
is alkyl, phenyl, substituted phenyl, or arylalkyl, and Rw is —CO, —CF₂, —CHOH, —CHOR′,
—C═CHCOOR, —(CH₂)₂OH, —CH₂CD₂OH, wherein R′ is alkyl, phenyl, substituted phenyl, or
arylalkyl.

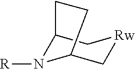

wherein Rw is —CHOH, —C(OH)R′, —C═O, —CHOR′, —(CH₂)₂OH, CH₂CD2OH, where R′ is alkyl,
arylalkyl, COOalkyl, SO2alkyl, or CONHalkyl, and R is H, alkyl, aryl, arylalkyl, substituted
aryl, (C═O)R′, (C═O)OR′, or SO₂R′, —CH₃SO₂, —PhCH₂CO, n-C₆H₁₁CO as well as a Cbz
(COOC(benzyl) or Boc protecting group (COOC(CH₃)₃), where R′ is an alkyl or arylalkyl.

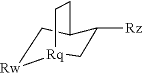

where Rw is —C(═O), —CHOH, —CF₂, —CH(OR′), —NHR′, —NR′R′, where R′ is an alkyl, phenyl,
substituted phenyl, arylalkyl, Rz is —OH, —COOH, —COOR′, —CH₂OH, —CD₂OH, and Rq
—CH₂OH, —CD₂OH, or N.

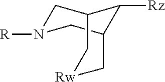

R is H, alkyl, phenyl, substituted phenyl, COOR′, SO₂R′, COR′, CONHR′, where R′ is an alkyl,
aryl (phenyl), or arylalkyl, and Rz is —OH, —CH₂OH, —CD₂OH, —COOH, —COOR′, —CONHR′,
—CONR′R′, and Rw is O, (S═O)ₙ where n is 0, 1, or 2, NR″, where R″ is COOR′, COR′, or
SO₂R′, wherein R′ is alkyl, phenyl, substituted phenyl, or arylalkyl.

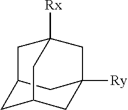

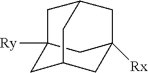

where each Rx is —OH, —CH₂OH, —CD₂OH, —(CH₂)₂OH, or —CH₂CD₂OH, and each Ry is —H,
—OH, —CH₂COOH, —(CH₂)₂OH, or —CH₂CD₂OH.

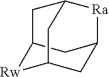

where Ra is —COOR, —CONHR′, —SO₂R′, or NR′, and Rw is —C═O, —CHOH, —CHOR′,
—(CH₂)₂OH, or —CH₂CD₂OH, where each R′ is an alkyl or arylalkyl.

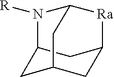

where each R is —COOR, —CONHR′, —SO₂R′, and each Ra is —C═O, —CHOH, —CHOR′,
—(CH₂)₂OH, or —CH₂CD₂OH, where each R′ is an alkyl or arylalkyl.

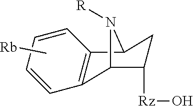

where each R is H, alkyl, arylalkyl, COOR′, SO2R′, COR′, CONHR′, where R′ is an alkyl or
arylalkyl, each Rz is —CH₂OH or —CD₂OH, and the aromatic ring can be substituted or
unsubstituted, e.g., where Rb is H, or a halogen (preferably F or Cl), or CN, or OR′, where R′
is an alkyl or aryl alkyl.

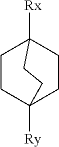

where Rx is —CH₂OH or —CD₂OH, and Ry is —COOR′, or —CONHR′ where R′ is an alkyl or
arylalkyl.
Pyrrolidine derivates (Series F), including hydroxy pyrrolidinones

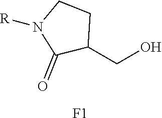

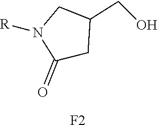

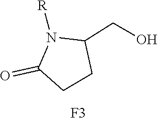

where R is H, alkyl, aryl, cycloalkene, arylalkyl, substituted aryl, (C═O)R′, (C═O)OR′, or
SO₂R′, —CH₃SO₂, CONHR′, —PhCH₂CO, n-C₆H₁₁CO, where R′ is an alkyl or arylalkyl. R can
also be a Cbz or Boc protecting group, which may be subsequently removed and derivatized to
one of the above substituents as described herein.
Carboxybenzyl (Cbz) Pyrrolidines (aka proline derivatives, L- or D- isomers possible)

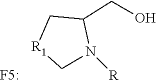

where R₁ is H or CF₂, and where R is H, alkyl, aryl, cycloalkene, arylalkyl, substituted aryl,
(C═O)R′, (C═O)OR′, or SO₂R′, —CH₃SO₂, CONHR′, —PhCH₂CO, n-C₆H₁₁CO, where R′ is an
alkyl or arylalkyl. R can also be a Cbz or Boc protecting group, which may be subsequently
removed and derivatized to one of the above substituents as described herein.

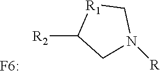

where R₁ is —CHOH, —CH(CH₂)₂OH, CHCH₂CD₂OH, and R₂ is alkyl, aryl alkyl, (C═O)R′,
(C═O)OR′, SO2R′, and R is H, alkyl, aryl, cycloalkene, arylalkyl, substituted aryl, (C═O)R′,
(C═O)OR′, or SO₂R′, —CH₃SO₂, CONHR′, —PhCH₂CO, n-C₆H₁₁CO, where R′ is an alkyl or
arylalkyl. R can also be a Cbz or Boc protecting group, which may be subsequently removed
and derivatized to one of the above substituents as described herein.

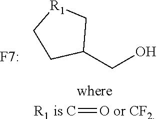

Phospholane derivatives

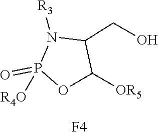

where R₃ is benzyl or isobutyl, R₄ is isopropyl, or 4-fluorobenzyl, and R₅ is H or D. Syn or anti
conformations of this structure are possible to access different regions in the S4 subsite, and can
be isolated separately after synthesis, or used as a mixture.
Azetidine derivatives (Series D)

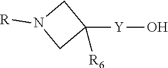

where each Y is (CRᵢRⱼ)ₙ, where n can be 0 (meaning Y is not present and the oxygen is directly
bonded to carbon of the ring) or 1-2 (meaning Y = CRᵢRⱼ where Rᵢ and Rⱼ can both be H or D, or
both methyl, or one H or D and the other methyl, and where R is H, alkyl, aryl, cycloalkene,
arylalkyl, substituted aryl, (C═O)R′, (C═O)OR′, or SO₂R′, —CH₃SO₂, CONHR′, —PhCH₂CO, n-
C₆H₁₁CO, where R′ is an alkyl or arylalkyl. R can be a Cbz or Boc protecting group, which
may be subsequently removed and derivatized to one of the above substituents as described
herein. R₆ is H, C₁-C₄ alkyl (preferably methyl), aryl, or alkylaryl (preferably benzyl).
Spirocycles (Series E)

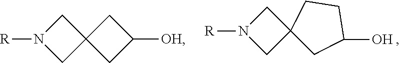

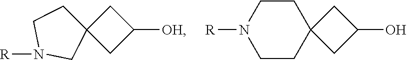

where each R is H, alkyl, aryl, cycloalkene, arylalkyl, substituted aryl, (C═O)R′, (C═O)OR′, or
SO₂R′, —CH₃SO₂, CONHR′, —PhCH₂CO, n-C₆H₁₁CO, where R′ is an alkyl or arylalkyl. R can
also be a Cbz or Boc protecting group, which may be subsequently removed and derivatized to
one of the above substituents as described herein. It is also contemplated that the hydroxy group
depicted above may be connected to the scaffold via a C₁-C₄ alkyl instead of directly attached
as shown.

Further embodiments contemplated herein for X design elements include conformationally-constrained piperidine bicycles:

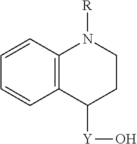

where Y is (CRᵢRⱼ)ₙ, where n can be 0 (meaning Y is not present and the oxygen is directly bonded to carbon of the ring) or 1-2 (meaning Y═CRᵢRⱼ where Rᵢ and Rⱼ can both be H or D, or both alkyl (e.g., methyl), or one H or D and the other alkyl (methyl)), and R is COOR, SO₂R, CONHR, alkyl, arylalkyl, substituted or unsubstituted phenyl, or CN. An exemplary synthesis of these compounds is illustrated below.

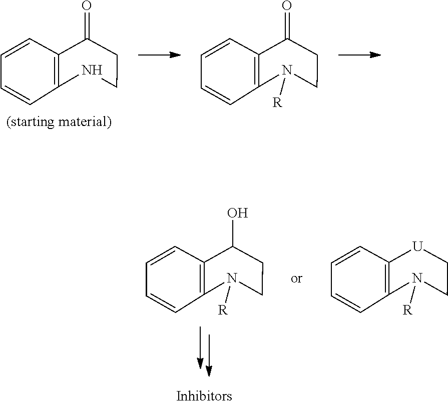

The initial step starts with reaction of the starting material scaffold with the desired derivative R group (e.g., as carboxylic acid, RSO₂Cl, phenyl, substituted phenyl, etc.), followed by mixing with 1-ethyl-3-(3-dimethylaminopropyl) carbodiimide (EDCI) to form the acid, and then treated with diisopropylethylamine (DIEA), using a traditional approach as described in U.S. Pat. No. 11,013,779, followed by reduction to form the alcohol, which can then be conjugated to the glutamine surrogate/side chain fragment using the new synthesis approach illustrated in FIG. 1.

Additional substituents for any of the foregoing X design elements may be defined further below with respect to specific compounds. Each of the foregoing structures is subject to the proviso that at least one group is an OH group for participation in the reaction described above. For example, at least one position in the structure contains an OH group, and when that position contains the primary or secondary alcohol for reaction, the other position(s) in the compound can be derivatized. As a nonlimiting example, an N-Boc protecting group can be removed during the synthesis process, to give a secondary amine, which can be derivatized by introducing an alkyl, arylalkyl, substituted aryl, C═O)alkyl, C═O)Oalkyl, SO₂alkyl, and the like, such that the other positions in the structure contain a substituent comprising an OH group (e.g., —CHOH, —C(OH)alkyl, —(CH₂)2OH, —CH₂CD₂OH, and the like. Similarly, typically when there is a nitrogen in the scaffold (e.g., piperidines or constrained piperidines, pyrrolidines or constrained pyrrolidines, etc.), it is preferably that the R group on the nitrogen will selected from H, alkyl, aryl, arylalkyl, substituted aryl, CN, (C═O)R′, (C═O)OR′, or SO₂R′, —CH₃SO₂, CONHR′, —PhCH₂CO, n-C₆H₁₁CO as well as a Cbz (COOC(benzyl) or Boc protecting group (COOC(CH₃)₃), where R′ is an alkyl or arylalkyl. As used herein, the term “alkyl” refers to straight chained and branched saturated hydrocarbon groups containing one to thirty carbon atoms, for example, one to twenty carbon atoms, or one to ten carbon atoms, preferably one to six carbon atoms. The term Cₙ means the alkyl group has “n” carbon atoms. For example, C₄ alkyl refers to an alkyl group that has 4 carbon atoms. C₁-C₆ alkyl refers to an alkyl group having a number of carbon atoms encompassing the entire range (e.g., 1 to 6 carbon atoms), as well as all subgroups (e.g., 1-6, 2-7, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl. Unless otherwise indicated, an alkyl group can be an unsubstituted alkyl group or a substituted alkyl group.

In some embodiments, particularly preferred X moieties include the following structures below connected to the backbone via the dashed line.

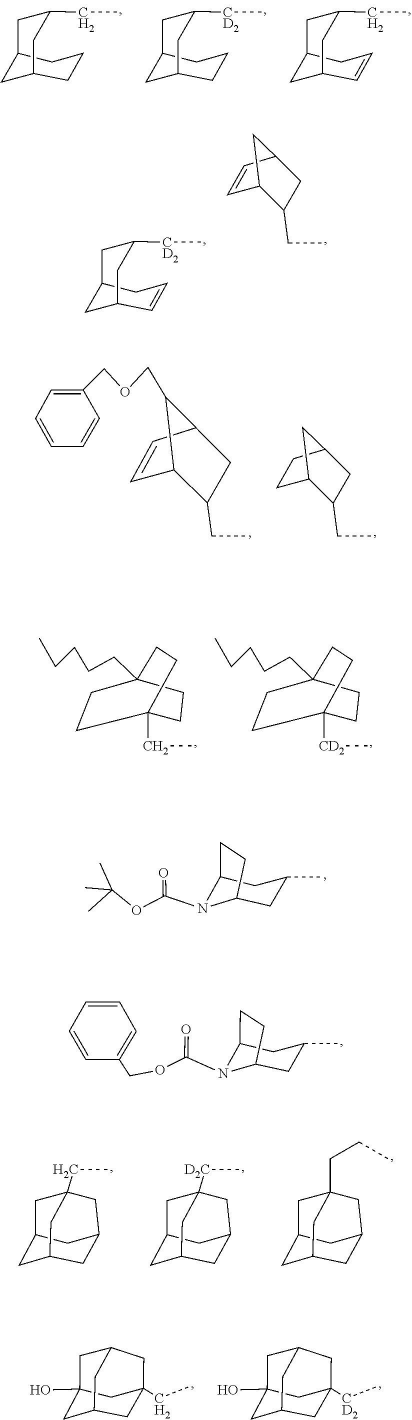

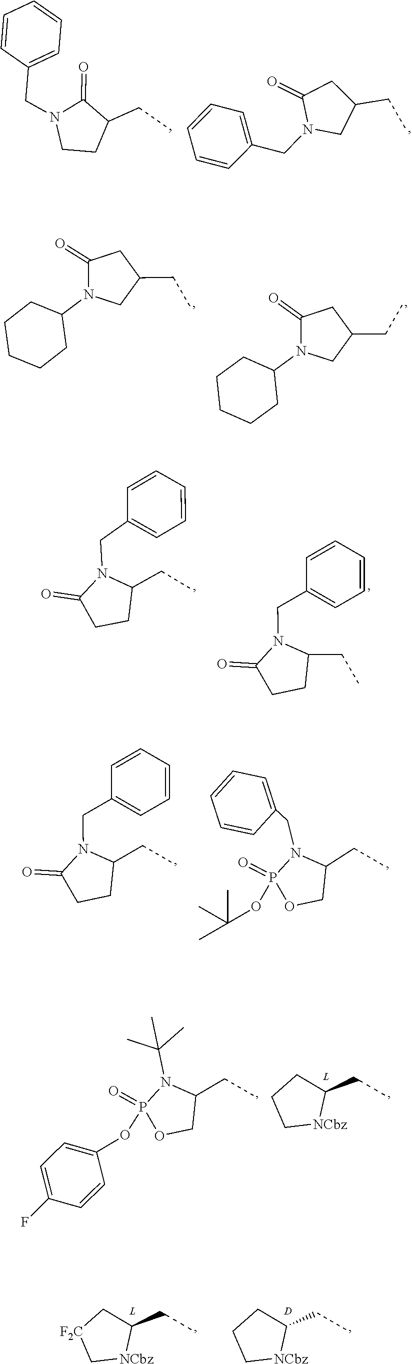

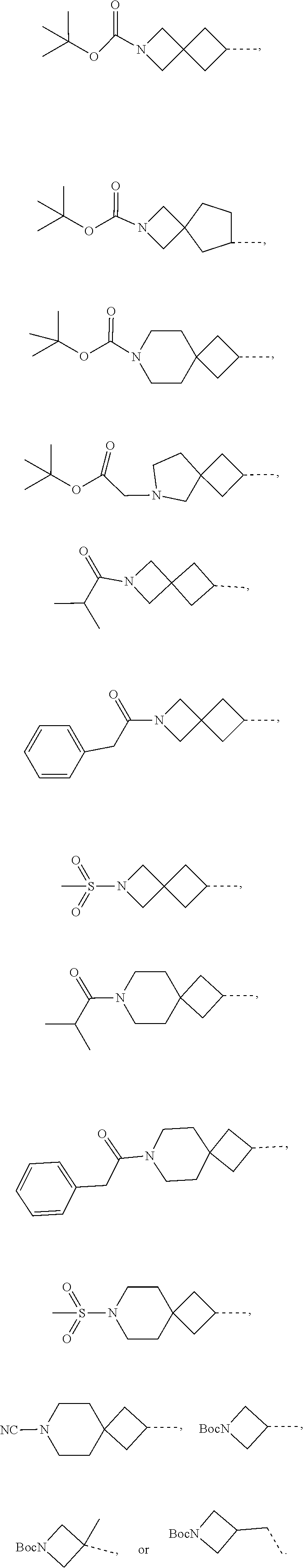

As noted elsewhere, it is contemplated that in any of the foregoing structures, the X moiety may be connected directly to the oxygen in the inhibitor backbone, or via a branched or unbranched alkyl linkage.

In one or more embodiments, using the foregoing backbone and alcohol input structures, exemplary aldehyde or bisulfite adduct inhibitors would thus comprise a structure such as:

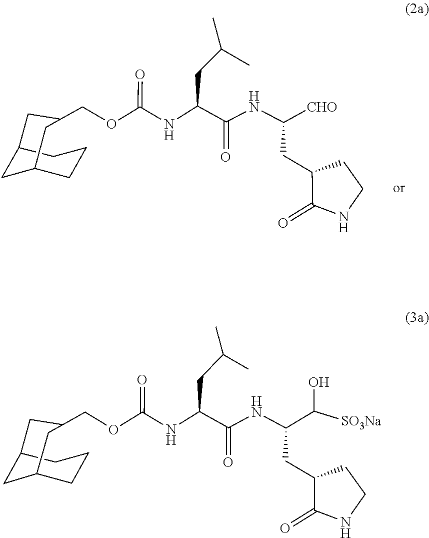

The present disclosure encompasses deuterated forms of the foregoing compounds, for example where hydrogen groups within the metabolically active sites of the X design element cyclic rings and/or in the adjacent carbon groups (e.g., methylene linkage) can be substituted with deuterium to generate deuterated variants of the compounds. It will be appreciated that pharmaceutically-acceptable salts of any of the compounds described here as well as their prodrug forms are also contemplated herein.

The term “pharmaceutically-acceptable salt” refers to an acid or base salt of a compound of the disclosure, which salt possesses the desired antiviral activity and is neither biologically nor otherwise undesirable. The present disclosure also includes prodrugs (ester, amide, carbamate, carbonate, ether, imine, phosphate, etc. derivatives) of the disclosed compounds. For example, the warhead, Z moiety, can be modified to generate prodrug forms of the compounds, which are described in detail in U.S. Pat. No. 11,033,600, incorporated by reference herein in its entirety.

Prophylactic and/or therapeutic compositions with specific or broad-spectrum antiviral activities are also disclosed. Combinations of one or more of the foregoing compounds are also contemplated. The compositions comprise an antiviral compound described herein dispersed in a pharmaceutically-acceptable carrier. The term carrier is used herein to refer to diluents, excipients, vehicles, and the like, in which the antiviral may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the compound or other agents and to minimize any adverse side effects in the subject. Pharmaceutically-acceptable ingredients include those acceptable for veterinary use as well as human pharmaceutical use, and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO) or other acceptable vehicles, and the like.

The composition can comprise a therapeutically effective amount of the compound dispersed in the carrier. As used herein, a “therapeutically effective” amount refers to the amount that will elicit the biological or medical response of a tissue, system, or subject that is being sought by a researcher or clinician, and in particular elicit some desired therapeutic or prophylactic effect as against the viral infection by slowing and/or inhibiting 3CL protease activity and/or viral replication. One of skill in the art recognizes that an amount may be considered therapeutically “effective” even if the condition is not totally eradicated or prevented, but it or its symptoms and/or effects are improved or alleviated partially in the subject. In some embodiments, the composition will comprise from about 5% to about 95% by weight of an antiviral compound described herein, and preferably from about 30% to about 90% by weight of the antiviral compound, based upon the total weight of the composition taken as 100% by weight. In some embodiments, combinations of more than one type of the described antiviral compounds can be included in the composition, in which case the total levels of all such compounds will preferably fall within the ranges described above.

Other ingredients may be included in the composition, such as adjuvants, other active agents, preservatives, buffering agents, salts, other pharmaceutically-acceptable ingredients. The term “adjuvant” is used herein to refer to substances that have immunopotentiating effects and are added to or co-formulated in a therapeutic composition in order to enhance, elicit, and/or modulate the innate, humoral, and/or cell-mediated immune response against the active ingredients. Other active agents that could be included in the composition include other antiviral compounds (e.g., cathepsins) or any immunogenic active components (e.g., antigens) such as those that resemble a disease-causing microorganism or infectious agent, and/or are made from weakened or killed forms of the same, its toxins, subunits, particles, and/or one of its surface proteins, such that it provokes an immune response to that microorganism or infectious agent. In addition to live, modified, or attenuated vaccine components, active agents using synthetic peptides, carbohydrates, or antigens can also be used.

Compositions according to the embodiments disclosed herein are useful in inhibiting protease activity. More specifically, the compositions can be used to inhibit viral infection or viral replication, such as by treating and/or preventing viral infection from a variety of causes, including caliciviruses (noroviruses), picornaviruses, and/or coronaviruses in a subject. Viruses in the picornavirus-like supercluster include important human and animal pathogens. For example, caliciviruses include noroviruses (Norwalk virus [NV]), feline calicivirus, MD145, murine norovirus [MNV], vesicular exanthema of swine virus, and rabbit hemorrhagic disease virus. Picornaviruses include enteroviruses (such as enterovirus 71), poliovirus, coxsackievirus, foot-and-mouth disease virus (FMDV), hepatitis A virus (HAV), porcine teschovirus, and rhinovirus (cause of common cold). Coronaviruses include human coronavirus (cause of common cold such as 229E strain), transmissible gastroenteritis virus (TGEV), murine hepatitis virus (MHV), bovine coronavirus (BCV), feline infectious peritonitis virus (FIPV), severe acute respiratory syndrome coronavirus (SARS-Co), SARS-CoV2 (causative agent of COVID-19), and Middle East respiratory syndrome coronavirus (MERS-CoV).

Compositions according to the embodiments disclosed herein are useful in treating and/or preventing viral infection from coronaviruses as well as against other viruses that belong to the picornavirus-like supercluster, including caliciviruses and picornaviruses in a subject. Thus, embodiments described herein have broad-spectrum therapeutic and/or prophylactic uses. The terms “therapeutic” or “treat,” as used herein, refer to processes that are intended to produce a beneficial change in an existing condition (e.g., viral infection, disease, disorder) of a subject, such as by reducing the severity of the clinical symptoms and/or effects of the infection, and/or reducing the duration of the infection/symptoms/effects. The terms “prophylactic” or “prevent,” as used herein, refer to processes that are intended to inhibit or ameliorate the effects of a future viral infection or disease to which a subject may be exposed (but is not currently infected with). In some cases, the composition may prevent the development of observable morbidity from viral infection (i.e., near 100% prevention). In other cases, the composition may only partially prevent and/or lessen the extent of morbidity due to the viral infection (i.e., reduce the severity of the symptoms and/or effects of the infection, and/or reduce the duration of the infection/symptoms/effects, or increase the rate of recovery from the condition). In either case, the compounds are still considered to “prevent” the target infection or disease.

In use, a therapeutically-effective amount of an antiviral compound is administered to a subject. In some embodiments, a composition comprising a therapeutically-effective amount of an antiviral compound is administered to a subject. Regardless, the compound or pharmaceutically acceptable salt thereof will preferably be administered to the subject in an amount sufficient to provide antiviral compound levels (independent of salt, if any) of from about 0.1 mg to about 1,000 mg of compound per kg of body weight of the subject, preferably from about 1 mg/kg to about 100 mg/kg of body weight of the subject, and more preferably from about 10 mg/kg to about 50 mg/kg of body weight of the subject. Thus, it will be appreciated that in the case of compound salts, for example, the formulation may be administered in amounts greater than the above ranges to provide sufficient levels of the active compound.

In some embodiments, the subject is afflicted with or suffering from a condition (e.g., infection, disease, or disorder) before the compounds are administered, wherein methods described herein are useful for treating the condition and/or ameliorating the effects of the condition. Preferably, the antiviral compound is administered as soon as possible after infection, preferably within about 7 days from onset of observable symptoms, more preferably within about 5 days from onset of observable symptoms, even more preferably within 3 days from onset of observable symptoms. It will be appreciated that the sooner the compound(s) is administered, the increased chance of successfully reducing effects of the viral infection. In other embodiments, the subject is free of a given condition before administering the compound, wherein the methods described herein are useful for preventing the occurrence or incidence of the condition and/or preventing the effects of the condition, as described above.

The disclosed embodiments are suitable for various routes of administration, depending upon the particular carrier and other ingredients used. For example, the prophylactic and/or therapeutic compounds or compositions can be injected intramuscularly, subcutaneously, intradermally, or intravenously. They can also be administered via mucosa such as intranasally or orally. The compounds or compositions can also be administered through the skin via a transdermal patch.

In some embodiments, the compound or compositions can be provided in unit dosage form in a suitable container. The term “unit dosage form” refers to a physically discrete unit suitable as a unitary dosage for human or animal use. Each unit dosage form may contain a predetermined amount of a compound disclosed herein (and/or other active agents) in the carrier calculated to produce a desired effect. In other embodiments, the compound can be provided separate from the carrier (e.g., in its own vial, ampule, sachet, or other suitable container) for on-site mixing before administration to a subject. A kit comprising the antiviral compound(s) is also disclosed herein. The kit further comprises instructions for administering the compound to a subject. The antiviral compound(s) can be provided as part of a dosage unit, already dispersed in a pharmaceutically-acceptable carrier, or it can be provided separately from the carrier. The kit can further comprise instructions for preparing the antiviral compounds for administration to a subject, including for example, instructions for dispersing the compounds in a suitable carrier.

It will be appreciated that therapeutic and prophylactic methods described herein are applicable to humans as well as any suitable animal, including, without limitation, dogs, cats, and other pets or captive animals (e.g., zoo animals, research subjects), as well as, rodents, primates, horses, cattle, pigs, etc. The methods can be also applied for clinical research and/or study. Additional advantages of the various embodiments of the disclosure will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present disclosure encompasses a variety of combinations and/or integrations of the specific embodiments described and claimed herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the disclosure. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration and nothing therein should be taken as a limitation upon the overall scope of the disclosure. Except where noted, precursor, intermediate, and final compounds described in the synthesis reactions below are independently numbered in each Example. Structures are indicated in the Tables below for avoidance of doubt.

### Introduction

Coronaviruses are enveloped, positive-sense, single-stranded RNA viruses that belong to the family Coronaviridae. Among human coronaviruses, several strains (229E, NL63, OC43, and KHU1) are the cause of mild upper respiratory infections; however, a few coronaviruses have emerged from animals that cause severe respiratory disease, including Severe Acute Respiratory Syndrome Coronavirus (SARS-CoV), Middle East Respiratory Syndrome Coronavirus (MERS-CoV) and Severe Acute Respiratory Syndrome Coronavirus-2 (SARS-CoV-2). Of particular concern is SARS-CoV-2, the highly pathogenic causative agent of COVID-19 which is associated with a high mortality rate and is a significant threat to public health worldwide. The problem is further compounded by the current lack of effective vaccines or small molecule therapeutics for the treatment of SARS-CoV-2 infections, underscoring the urgent and dire need for the development of prophylactic and therapeutic countermeasures to combat infections by pathogenic coronaviruses.

The SARS-CoV-2 genome is large (˜30 kb) and similar to the genomes of SARS-CoV and MERS-CoV (˜80% and ˜50% sequence identity, respectively). It contains two open reading frames (ORF1a and ORF1b) and encodes multiple structural and nonstructural proteins. Translation of the genomic mRNA of ORF1a yields a polyprotein (pp1a), while a second polyprotein (pp1b) is the product of a ribosomal frameshift that joins ORF1a together with ORF1b. The two polyproteins are processed by a 3C-like protease (3CLpro, also referred to as Main protease, M^{rpo}) (11 cleavage sites) and a papain-like cysteine protease (PLpro), resulting in 16 mature nonstructural proteins which are involved in the replication-transcription complex. The two proteases are essential for viral replication, making them attractive targets for therapeutic intervention.

SARS-CoV-2 3CLpro is a homodimer with a catalytic Cys-His dyad (Cys¹⁴⁵-His⁴¹) and an extended binding cleft. Substrate specificity profiling studies have shown that the protease displays a strong preference for a —Y—Z-Leu-Gln-X sequence, where X is a small amino acid, Y is a hydrophobic amino acid, and Z is solvent-exposed and fairly diverse (V/T/K), corresponding to the subsites —S₄-S₃—S₂—S₁-S₁′—. Cleavage is at the P₁-P₁′ scissile bond. The 3D structure of SARS-CoV-2 3CLpro is similar to that of SARS-CoV 3CLpro, however, the S₂ subsite of SARS-CoV-2 3CLpro displays considerable plasticity and can accommodate natural and unnatural amino acids with smaller side chains. High-resolution crystal structures with bound inhibitors have been determined, enabling the use of structure-guided approaches in the design of inhibitors. In continuing our foray in this area, we report herein the results of preliminary studies related to the inhibition of SARS-CoV-2 protease by a series of inhibitors (I) that incorporate in their structure a conformationally-constrained cyclohexane moiety envisaged to exploit new chemical space and to optimally engage in favorable binding interactions with the active site of the protease.

Inhibitor Design Rationale. The design of inhibitor (I) (Scheme) included the use of a P₁ glutamine surrogate residue and a P₂ Leu residue as recognition elements congruent with the substrate specificity of the protease, as well as an aldehyde warhead or a latent aldehyde bisulfite adduct. The design of inhibitor (I) was further abetted by insights gained from examining the available X-ray crystal structures of the protease with inhibitors.

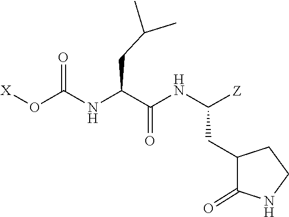

### Example 1

In this Example, a series of non-deuterated and deuterated dipeptidyl aldehyde and masked aldehyde inhibitors (2 (a-o)) and bisulfite adducts thereof (3 (a-o)) that incorporate in their structure a conformationally-constrained cyclohexane moiety was synthesized and found to potently inhibit SARS-CoV-2 3CL protease in biochemical and cell-based assays. Several of the inhibitors were also found to be nanomolar inhibitors of MERS-CoV 3CL protease. The corresponding latent aldehyde bisulfite adducts were found to be equipotent to the precursor aldehydes. High-resolution cocrystal structures confirmed the mechanism of action and illuminated the structural determinants involved in binding. The spatial disposition of the compounds disclosed herein provides an effective means of accessing new chemical space and optimizing pharmacological activity. The cellular permeability of the identified inhibitors and lack of cytotoxicity warrants their advancement as potential therapeutics for COVID-19.

### Results and Discussion

### Inhibitor Design Rationale

The design of the inhibitors (FIG. 1) included the use of a P₁ glutamine surrogate residue and a P₂ Leu residue as recognition elements congruent with the substrate specificity of the protease, as well as an aldehyde warhead or a latent aldehyde bisulfite adduct. The design of inhibitor (I) was further abetted by insights gained from examining the available X-ray crystal structures of the protease with inhibitors and the results of recent studies with cyclohexyl-derived inhibitors with demonstrated efficacy in a mouse model of MERS-CoV-2 infection and potent inhibition against SARS-CoV-2 3CL protease.

### Chemistry

The synthesis of inhibitors 2(a-o) and 3(a-o) was readily accomplished by activating the precursor primary or secondary alcohol inputs (Table 2) with N,N′-disuccinimidyl carbonate (DSC) and coupling the mixed carbonate with the readily-accessible Leu-Gln surrogate amino alcohol to yield alcohol product 1 which was oxidized with Dess-Martin periodinane (DMP) to generate the corresponding aldehyde 2 (FIG. 1, Scheme 1, Z═CHO). The aldehydes were subsequently converted to the corresponding bisulfite adducts 3 (FIG. 1, Scheme 1, Z═CH(OH)SO₃Na)

TABLE 2
Series B Alcohol Inputs for derivatives of inhibitors 2 (aldehyde) and
3 (bisulfite adducts)

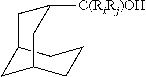

| Compound | Ri | Rj |
|---|---|---|
| a | H | H |
| b | D | D |

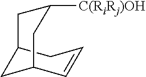

| Compound | Ri | Rj |
|---|---|---|
| c | H | H |
| d | D | D |

| Compound e | 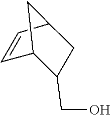 |
|---|---|
| Compound f | 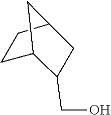 |

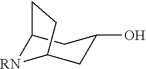

| Compound | R |
|---|---|
| g | t-Boc |
| h | Cbz |

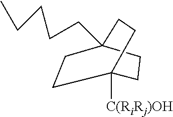

| Compound | Ri | Rj |
|---|---|---|
| i | H | H |
| j | D | D |

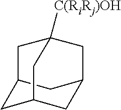

| Compound | Ri | Rj |
|---|---|---|
| k | H | H |
| l | D | D |

| | |
|---|---|
| Compound m | 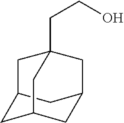 |

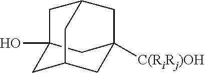

| Compound | Ri | Rj |
|---|---|---|
| n | H | H |
| o | D | D |

### Biochemical Studies

The inhibitory activity of the aldehydes (compounds 2 (a-o)) and bisulfite adducts (compounds 3 (a-o)) against SARS-CoV-2 3CL protease and their activity in a cell-based system, were determined as described in the experimental section. The IC₅₀ values (50% inhibitory concentration in the enzyme assay); EC₅₀ values (50% effective concentration in cell culture) for two representative inhibitors (2a/3a), and the CC₅₀ values (50% cytotoxic concentration in cell-based assays) in Huh-7, CRFK, or CCL1 cells are summarized in Table 3 and they are the average of at least two determinations. The inhibitory activity of compounds 2a/3a, 2f/3f, and 2k/3k against IVERS-CoV 3CL protease was also determined as described previously and the IC₅₀ values are listed in Table 4.

TABLE 3
IC₅₀ and CC₅₀ values of SARS-CoV-2 3CLpro inhibitors 2-3(a-o)

| Compound | IC₅₀ (μM) | CC₅₀ (μM) |
|---|---|---|
| 2a | 0.18 ± 0.03^{a} | >100 |
| 3a | 0.17 ± 0.02^{a} | >100 |
| 2b | 0.29 ± 0.05 | >100 |
| 3b | 0.29 ± 0.01 | >100 |
| 2c | 0.31 ± 0.06 | >100 |
| 3c | 0.26 ± 0.05 | >100 |
| 2d | 0.28 ± 0.08 | >100 |
| 3d | 0.30 ± 0.03 | >100 |
| 2e | 0.26 ± 0.05 | >100 |
| 3e | 0.28 ± 0.03 | >100 |
| 2f | 0.14 ± 0.02 | >100 |
| 3f | 0.10 ± 0.01 | >100 |
| 2g | 1.90 ± 0.14 | >100 |
| 3g | 1.71 ± 0.16 | >100 |
| 2h | 0.39 ± 0.02 | >100 |
| 3h | 0.37 ± 0.03 | >100 |
| 2i | 0.27 ± 0.02 | 21 ± 1 |
| 3i | 0.30 ± 0.02 | 21 ± 3 |
| 2j | 0.31 ± 0.03 | 20 ± 3 |
| 3j | 0.31 ± 0.06 | 23 ± 1 |
| 2k | 0.18 ± 0.04 | >100 |
| 3k | 0.15 ± 0.04 | >100 |
| 2l | 0.29 ± 0.04 | >100 |
| 3l | 0.27 ± 0.04 | >100 |
| 2m | 0.27 ± 0.02 | >100 |
| 3m | 0.25 ± 0.04 | >100 |
| 2n | 0.82 ± 0.19 | >100 |
| 3n | 1.03 ± 0.24 | >100 |
| 2o | 0.74 ± 0.26 | >100 |
| 3o | 0.78 ± 0.18 | >100 |

^{a}The EC₅₀ values for inhibitors 2a and 3a against SARS-CoV-2 in Vero E6 cells were 0.035 ± 0.001 and 0.032 ± 0.001 μM, respectively.

TABLE 4
IC₅₀ values of MERS-CoV 3CLpro inhibitors
2a/3a, 2f/3f and 2k/3k.

| Compound | IC₅₀ (μM) |
|---|---|
| 2a | 0.052 ± 0.001 |
| 3a | 0.049 ± 0.002 |
| 2f | 0.063 ± 0.003 |
| 3f | 0.058 ± 0.002 |
| 2k | 0.055 ± 0.002 |
| 3k | 0.053 ± 0.003 |

It is clearly evident from the results shown in Table 3 that the synthesized compounds display high potency in biochemical assays, with most IC₅₀ values in the sub-micromolar range. Furthermore, the inhibitors were found to be devoid of cytotoxicity and the Safety Index (SI), defined as the CC₅₀/IC₅₀ ratio, ranged between ˜78 to 1110. The potency of deuterated variants 2b/3b decreased ˜1.6-fold (aldehydes) and ˜1.7-fold (bisulfite adducts) as compared to the respective non-deuterated compounds 2a/3a and remained essentially the same in the case of non-deuterated 2n/3n and deuterated 2o/3o inhibitors, respectively. A change in geometry from a cyclohexene (2e/3e) to a cyclohexane (2f/3f) resulted in a 2 to 3-fold increase in potency. The ˜5-fold decrease in potency of compounds 2n/3n compared to 2k/3k presumably reflects the inimical effect on potency of the 3° hydroxyl group. Importantly, the EC₅₀ values of two representative inhibitors (2a/3a) against SARS-CoV-2 in Vero E6 cells were found to be ˜4.6-fold lower (EC₅₀ 0.035 and 0.032 μM, respectively) than the corresponding IC₅₀ values, and the selectivity indices of compounds 2a/3a were very high (2857 and 3125, respectively). The significance of these findings was further augmented by the notable inhibition of MERS-CoV 3CL protease by a select number of inhibitors (Table 3, compounds 2a/3a, 2f/3f and 2k/3k), demonstrating the broad spectrum of antiviral activity displayed by this series of compounds.

Emergence of viral resistance to antiviral drugs is a major concern. We previously reported that GC376 has a high barrier to resistance to feline infectious peritonitis virus (FIPV) in cell culture and naturally infected animals with long term treatment. We also examined several compounds similar to the series in this report for emergence of viral resistance by serial passaging FIPV in the presence of each compound in cell culture. The EC₅₀ values of the compounds did not increase at up to 10 passage number, and the 3CLpro of viruses passaged with each compound has the same sequence as mock-passaged viruses. These results suggest that this series of compounds have a high barrier to resistance.

### X-Ray Crystallography Studies

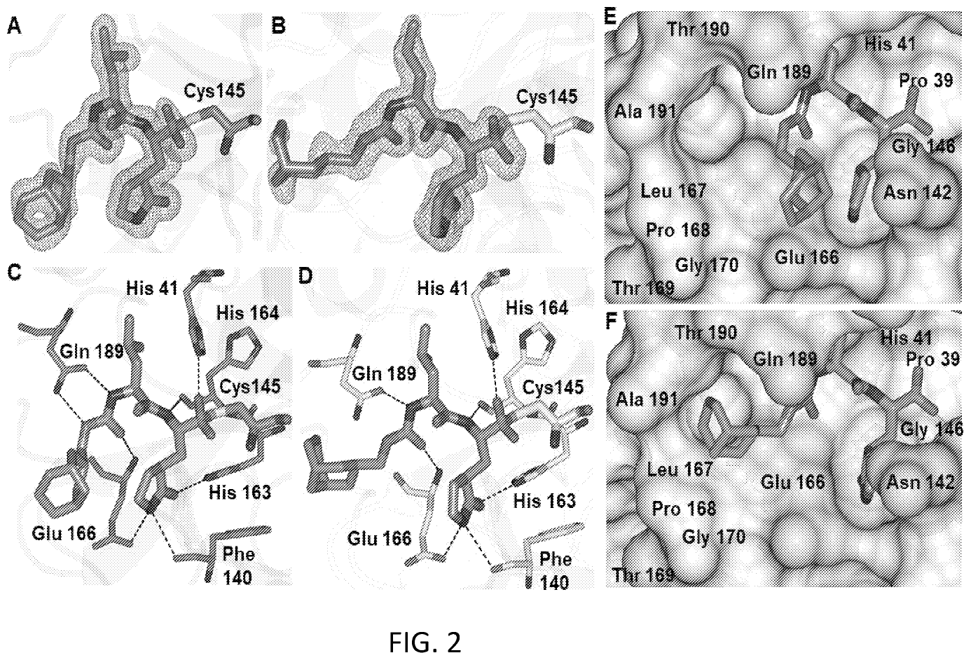

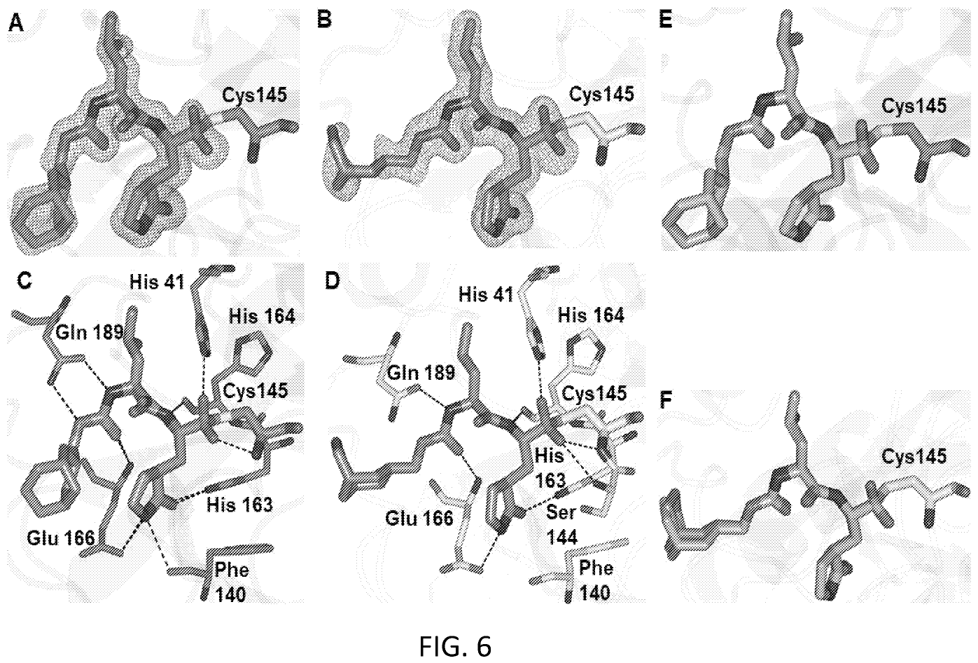

In order to elucidate the mechanism of action of the inhibitors as well as identify the structural determinants associated with the binding of inhibitors to the active site of SARS-CoV-2 3CL protease, high-resolution cocrystal structures were determined for inhibitors 2a and its deuterated analog 3b, 2f, 2k, 3c and its deuterated analog 3d and 3e. The structure of SARS-CoV-2 3CLpro in complex with compound 2a contained prominent difference electron density consistent with the inhibitor covalently bound to Cys 145 in each subunit (FIGS. 2A & B). The electron density was consistent with the inhibitor aldehyde carbon covalently bound to the Sγ atom of the catalytic Cys145 residue and the formation of a tetrahedral hemithioacetal, confirming the mechanism of action. Both the R and S-enantiomers were observed at the newly formed stereocenter and each enantiomer was modeled with 0.5 occupancy and was observed for all structures described here. Interestingly, 2a adopts two conformations in which the bicyclic ring is projected away from the S4 subsite in subunit A and is positioned in the S4 pocket in subunit B. The inhibitor engages in multiple favorable binding interactions with the enzyme, including direct hydrogen bond interactions with His163 and Glu166 (γ-lactam C═O and N—H, respectively), His41, Phe 140, Ser144, His164, and Gln189 (FIGS. 2C & D). The isobutyl side chain of Leu is ensconced in the hydrophobic S₂ pocket and the γ-lactam ring of the P₁ Gln surrogate is nestled in the S₁ subsite forming hydrogen bonds with His163 and Glu166. In addition, the lipophilic bicyclic ring in subunit A is directed towards the surface whereas in subunit B is anchored in the vicinity of the hydrophobic S₄ pocket that is lined by Ala191, Leu197, and Pro168 (FIGS. 2E & F). It should be noted that the eleven sites in the pp1a and pp1ab polyproteins cleaved by the protease are all characterized by the presence of a P₁ Gln residue, which is conserved in all known coronavirus 3CLpro cleavage sites. Interestingly, the deuterated analog 3b adopts the same binding mode and superimposes nearly identical to 2a as shown in FIG. 6. The root mean square deviation (RMSD) between the C-alpha atoms of 2a and 3b was 0.27 Å for 594 residues aligned.

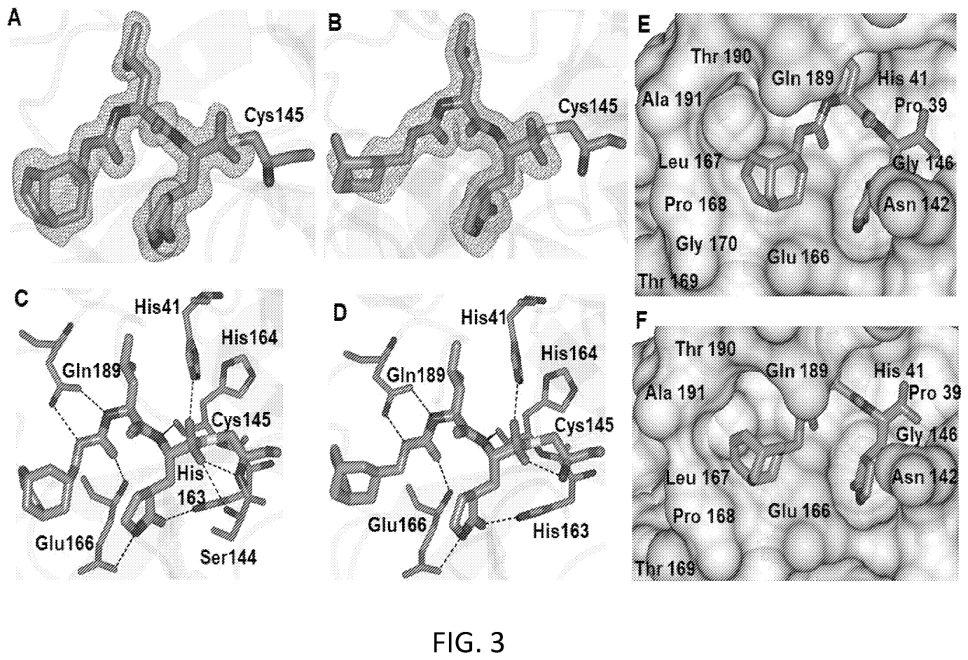

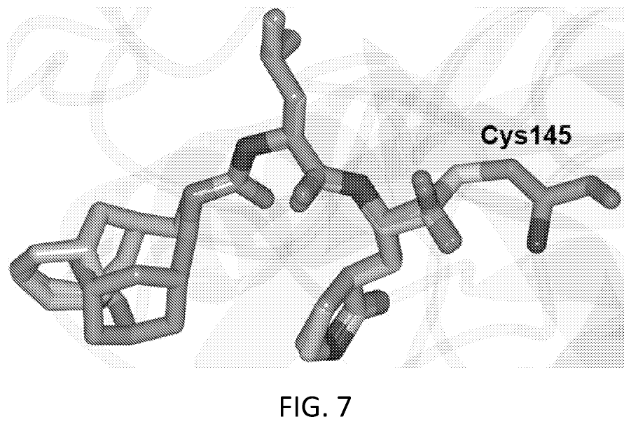

Likewise, the structure of 3c shows similar binding mode properties as observed for 2a (FIGS. 3A, C & E). The inhibitor engages in multiple favorable binding interactions with the enzyme, including direct hydrogen bond interactions with His163 and Glu166 (γ-lactam C═O and N—H, respectively), His41, Ser144, His164, and Gln189. The bicyclic ring is oriented within the hydrophobic S₄ pocket, in both subunits. Likewise, the structure of SARS-CoV-2 3CL protease with deuterated inhibitor 3d adopts a very similar binding mode (FIGS. 3 C, D & F). The main difference is that the electron density for 3c is most consistent with an axial conformation of the carbon atom attached to the bicyclic ring whereas 3d appears to adopt an equatorial orientation (FIG. 7). The structures are very similar overall and the superposition yielded an RMSD deviation between the C-alpha atoms of 0.25 Å for 596 residues aligned.

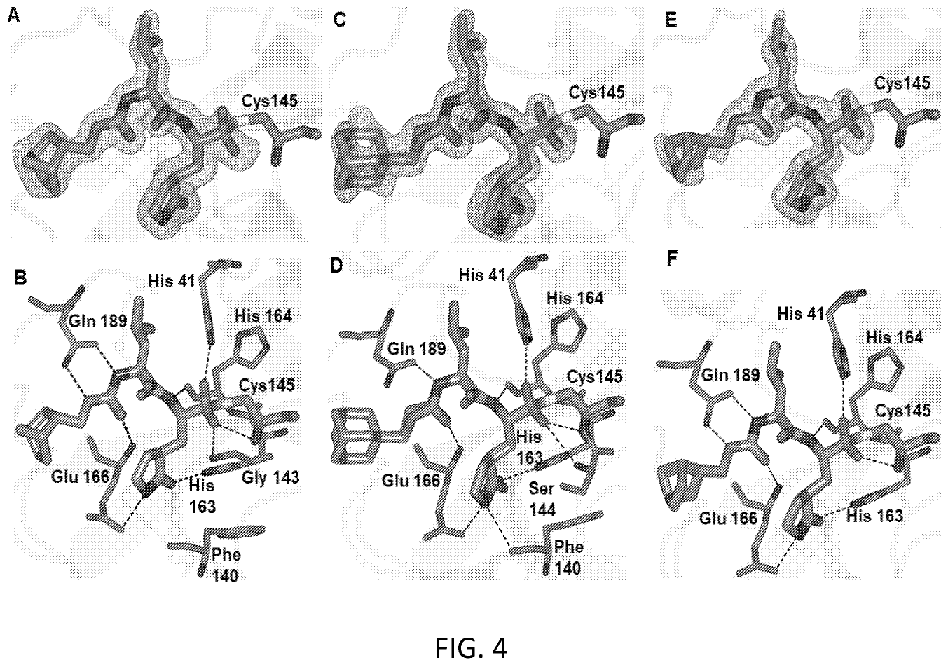

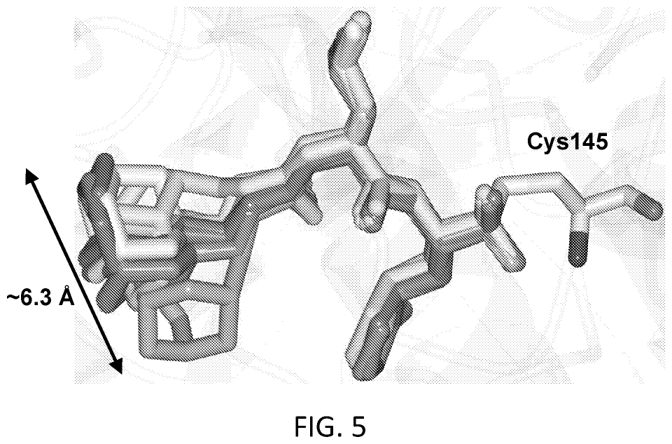

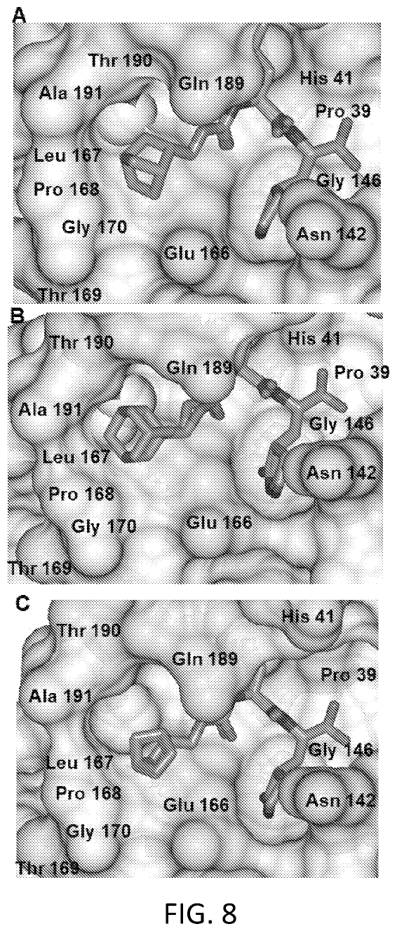

Similarly, inhibitors 2f, 2k and 3e in complex with SARS-CoV-2 were found to adopt similar binding modes in the active site of the protease as shown in FIG. 4 and FIG. 8. Collectively, the bicyclic rings of the inhibitors span a relatively small region in the active site and cover a space of approximately 6.3 Å (FIG. 5). As such, these cocrystal structures provide valuable insights for further structure-guided multiparameter optimization.

### Conclusions

Given the major clinical importance associated with the SARS-CoV-2 pandemic and the current paucity of effective countermeasures, the results of the studies described herein can serve as a launching pad for conducting further pre-clinical studies. Most of the compounds exhibited high potency in biochemical assays and, for two of the compounds tested, in cellular assays. Furthermore, members of this series were also found to potently inhibit MERS-CoV 3CL protease, suggesting that the compounds can be developed into broad-spectrum antivirals. Since there are no known human proteases that have a primary substrate specificity P₁ residue that is Gln, these inhibitors could also display high selectivity and diminished off-target effects. Furthermore, the utilization of an aldehyde warhead, or a latent aldehyde functionality that can rapidly generate the aldehyde in vivo, in the design of transition state inhibitors is advantageous for several reasons, including rapid engagement with the target leading to the reversible formation of a covalent adduct. The high reactivity of aldehydes is generally viewed as a toxicity alert, however, the safety indices for most of the compounds reported herein were found to be high. Indeed, a number of pharmaceuticals that incorporate in their structure an aldehyde functionality are currently in clinical use and, furthermore, toxicity arising from the presence of the aldehyde is context-specific, as is presumably the case here. Finally, the present study also sought to exploit the kinetic isotope effect associated with the H/D bioisosteric replacement in order to dampen oxidative metabolism at the —CH₂O— metabolic soft spot in the inhibitors, as well as to reduce toxicity. Thus, the availability of equipotent deuterated analogs that display improved pharmacokinetics (PK) characteristics enhances further the significance of the results reported herein. Evaluation of a select number of inhibitors in a mouse model of SARS-CoV-2 infection is in progress and the results will be reported in due course. In conclusion, a series of potent transition state inhibitors of SARS-CoV-2 3CL protease that incorporate in their structures a conformationally-constrained cyclohexyl moiety is reported.

### Experimental Section

### General

Reagents and dry solvents were purchased from various chemical suppliers (Sigma-Aldrich, Acros Organics, Chem-Impex, TCI America, Oakwood chemical, APExBIO, Cambridge Isotopes, Alpha Aesar, Fisher and Advanced Chemblocks) and were used as obtained. Silica gel (230-450 mesh) used for flash chromatography was purchased from Sorbent Technologies (Atlanta, GA). Thin layer chromatography was performed using Analtech silica gel plates. Visualization was accomplished using UV light and/or iodine. NMR spectra were recorded in CDCl₃ or DMSO-d₆ using Varian XL-400 spectrometer. Melting points were recorded on a Mel-Temp apparatus and are uncorrected. High resolution mass spectrometry (HRMS) was performed at the Wichita State University Mass Spectrometry lab using Orbitrap Velos Pro mass spectrometer (ThermoFisher, Waltham, MA) equipped with an electrospray ion source. The purity of all final compounds was >95% as evidenced by NMR analysis.

### Synthesis of Compounds

Preparation of compounds 1(a-o). General procedure. As illustrated in FIG. 1, to a solution of alcohol (1 eq) (Table 2) in anhydrous acetonitrile (10 mL/g alcohol) was added N,N′-disuccinimidyl carbonate (1.2 eq) and triethyl amine (TEA) (3.0 eq) and the reaction mixture was stirred for 4 h at room temperature. The solvent was removed in vacuo and the residue was dissolved in ethyl acetate (40 mL/g alcohol). The organic phase was washed with saturated aqueous NaHCO₃(2×20 mL/g alcohol), followed by brine (20 mL/g alcohol). The organic layers were combined and dried over anhydrous Na₂SO₄, filtered and concentrated in vacuo to yield the mixed carbonate which was used in the next step without further purification.

To a solution of Leu-Gln surrogate amino alcohol (1.0 eq) in dry methylene chloride (10 mL/g of amino alcohol) was added TEA (1.5 eq) and the reaction mixture was stirred for 20 min at room temperature (solution 1). In a separate flask, the mixed carbonate was dissolved in dry methylene chloride (10 mL/g of carbonate) (solution 2). Solution 1 was added to solution 2 and the reaction mixture was stirred 3 h at room temperature. Methylene chloride was added to the organic phase (40 mL/g of carbonate) and then washed with saturated aqueous NaHCO₃(2×20 mL/g alcohol), followed by brine (20 mL/g alcohol). The organic phase was dried over anhydrous Na₂SO₄, filtered and concentrated in vacuo. The resultant crude product was purified by flash chromatography (hexane/ethyl acetate) to yield the dipeptidyl alcohol 1 for each respective precursor, as a white solid.

((1R,5S)-Bicyclo[3.3.1]nonan-3-yl)methyl ((S)-1-(((S)-1-hydroxy-3-((S)-2-oxopyrrolidin-3-yl)propan-2-yl)amino)-4-methyl-1-oxopentan-2-yl)carbamate (1a). Yield (3 5%); ¹H NMR (400 MHz, CDCl₃) δ 7.75 (d, J=7.2 Hz, 1H), 6.24 (s, 1H), 5.31 (d, J=9.1 Hz, 1H), 4.29-4.10 (m, 1H), 4.10-3.93 (m, 1H), 3.89 (d, J=6.3 Hz, 1H), 3.71-3.54 (m, 2H), 3.48 (d, J=2.0 Hz, 1H), 3.39-3.27 (m, 2H), 2.55-2.25 (m, 3H), 2.03 (d, J=11.9 Hz, 3H), 1.96-1.77 (m, 4H), 1.77-1.57 (m, 4H), 1.57-1.44 (m, 1H), 1.37 (d, J=9.5 Hz, 4H), 1.27-1.15 (m, 1H), 1.09 (dd, J=12.8, 2.6 Hz, 1H), 0.95 (d, J=6.3 Hz, 6H), 0.91-0.80 (m, 2H).

((1R,3S,5S)-Bicyclo[3.3.1]nonan-3-yl)methyl-d2 ((S)-1-(((S)-1-hydroxy-3-((S)-2-oxopyrrolidin-3-yl)propan-2-yl)amino)-4-methyl-1-oxopentan-2-yl)carbamate (1b). Yield (36%); ¹H NMR (400 MHz, CDCl₃) δ 7.77 (d, J=7.1 Hz, 1H), 6.25 (s, 1H), 5.30 (s, 1H), 4.27-3.93 (m, 2H), 3.69-3.55 (m, 2H), 3.39-3.32 (m, 2H), 2.53-2.35 (m, 2H), 2.09-1.96 (m, 4H), 1.96-1.77 (m, 3H), 1.77-1.58 (m, 5H), 1.58-1.47 (m, 1H), 1.45-1.30 (m, 5H), 1.09 (d, J=12.8 Hz, 1H), 0.95 (d, J=6.4 Hz, 6H), 0.88 (d, J=13.1 Hz, 2H).

((1S,5R)-Bicyclo[3.3.1]non-6-en-3-yl)methyl((S)-1-(((S)-1-hydroxy-3-((S)-2-oxopyrrolidin-3-yl)propan-2-yl)amino)-4-methyl-1-oxopentan-2-yl)carbamate (1c). Yield (3 5%). ¹H NMR (400 MHz, CDCl₃) δ 7.74 (s, 1H), 5.98 (s, 1H), 5.85 (t, J=8.0 Hz, 1H), 5.58 (t, J=9.7 Hz, 1H), 5.19 (s, 1H), 4.21-4.16 (m, 1H), 4.07 (d, J=2.5 Hz, 2H), 4.02-3.95 (m, 1H), 3.68-3.56 (m, 2H), 3.38-3.32 (m, 2H), 2.50-2.36 (m, 2H), 2.36-2.31 (m, 1H), 2.31-2.25 (m, 2H), 2.17-2.09 (m, 1H), 2.03-1.95 (m, 1H), 1.95-1.87 (m, 1H), 1.87-1.81 (m, 2H), 1.81-1.73 (m, 2H), 1.73-1.59 (m, 3H), 1.56-1.46 (m, 2H), 1.46-1.35 (m, 2H), 0.95 (d, J=6.5 Hz, 6H).

((1S,3S,5R)-Bicyclo[3.3.1]non-6-en-3-yl)methyl-d2((S)-1-(((S)-1-hydroxy-3-((S)-2-oxopyrrolidin-3-yl)propan-2-yl)amino)-4-methyl-1-oxopentan-2-yl)carbamate (1d). Yield (36%). ¹H NMR (400 MHz, CDCl₃) δ 7.72 (s, 1H), 6.04 (s, 1H), 5.85 (t, J=8.1 Hz, 1H), 5.62-5.55 (m, 1H), 5.20 (s, 1H), 4.23-4.15 (m, 1H), 4.02-3.93 (m, 1H), 3.68-3.54 (m, 2H), 3.39-3.33 (m, 2H), 2.49-2.31 (m, 3H), 2.30-2.26 (m, 1H), 2.16-2.09 (m, 1H), 2.03-1.95 (m, 2H), 1.93-1.78 (m, 4H), 1.78-1.59 (m, 4H), 1.59-1.46 (m, 2H), 1.46-1.35 (m, 2H), 0.95 (d, J=6.5 Hz, 6H).

Bicyclo[2.2.1]hept-5-en-2-ylmethyl ((S)-1-(((S)-1-hydroxy-3-((S)-2-oxopyrrolidin-3-yl)propan-2-yl)amino)-4-methyl-1-oxopentan-2-yl)carbamate (1e). Yield (44%). ¹H NMR (400 MHz, CDCl₃) δ 7.74 (s, 1H), 6.38 (s, 1H), 6.14 (dd, J=5.7, 3.0 Hz, 1H), 5.93 (dd, J=5.8, 2.9 Hz, 1H), 5.41-5.32 (m, 1H), 4.27-4.19 (m, 1H), 4.19-4.07 (m, 1H), 4.06-3.89 (m, 1H), 3.88-3.80 (m, 1H), 3.69-3.53 (m, 3H), 3.35 (dd, J=10.6, 4.2 Hz, 2H), 2.87-2.77 (m, 2H), 2.52-2.34 (m, 2H), 2.10-1.87 (m, 1H), 1.87-1.77 (m, 1H), 1.77-1.57 (m, 3H), 1.57-1.47 (m, 1H), 1.47-1.40 (m, 1H), 1.37-1.18 (m, 1H), 1.18-1.10 (m, 1H), 0.96 (d, J=6.5 Hz, 6H), 0.53 (ddd, J=11.7, 4.5, 2.6 Hz, 1H).

Bicyclo[2.2.1]heptan-2-ylmethyl ((S)-1-(((S)-1-hydroxy-3-((S)-2-oxopyrrolidin-3-yl)propan-2-yl)amino)-4-methyl-1-oxopentan-2-yl)carbamate (1f). Yield (44%). ¹H NMR (400 MHz, CDCl₃) δ 7.73 (s, 1H), 6.30 (s, 1H), 5.30 (s, 1H), 4.30-4.15 (m, 1H), 4.15-4.04 (m, 1H), 4.04-3.95 (m, 1H), 3.95-3.85 (m, 1H), 3.78 (d, J=8.0 Hz, 1H), 3.71-3.52 (m, 2H), 3.39-3.32 (m, 2H), 2.48-2.36 (m, 2H), 2.24-2.17 (m, 1H), 2.17-1.96 (m, 1H), 1.97-1.75 (m, 1H), 1.75-1.56 (m, 3H), 1.56-1.41 (m, 3H), 1.41-1.21 (m, 3H), 1.21-0.99 (m, 3H), 0.95 (d, J=6.4 Hz, 6H), 0.66 (dd, J=12.6, 5.1 Hz, 1H).

tert-Butyl (1R,3s,5S)-3-((((S)-1-(((S)-1-hydroxy-3-((S)-2-oxopyrrolidin-3-yl)propan-2-yl)amino)-4-methyl-1-oxopentan-2-yl)carbamoyl)oxy)-8-azabicyclo[3.2.1]octane-8-carboxylate (1g). Yield (45%). ¹H NMR (400 MHz, CDCl₃) δ 7.85 (s, 1H), 6.29 (s, 1H), 5.31 (d, J=9.3 Hz, 1H), 4.94 (s, 1H), 4.29-4.02 (m, 3H), 4.01-3.97 (m, 1H), 3.64-3.59 (m, 2H), 3.39-3.31 (m, 2H), 2.44-2.39 (m, 2H), 2.20-1.89 (m, 8H), 1.84 (dd, J=11.3, 9.0 Hz, 1H), 1.76 (d, J=15.2 Hz, 2H), 1.73-1.60 (m, 2H), 1.60-1.48 (m, 1H), 1.46 (s, 9H), 0.99-0.91 (m, 6H).

Benzyl (1R,3s,5S)-3-((((S)-1-(((S)-1-hydroxy-3-((S)-2-oxopyrrolidin-3-yl)propan-2-yl)amino)-4-methyl-1-oxopentan-2-yl)carbamoyl)oxy)-8-azabicyclo[3.2.1]octane-8-carboxylate (1h). Yield (49%). ¹H NMR (400 MHz, DMSO-d₆) δ 7.60-7.56 (m, 2H), 7.47-7.10 (m, 6H), 5.11 (d, J 10.53 Hz, 2H), 5.02-4.88 (m, 1H), 4.83-4.61 (m, 1H), 4.30-4.15 (m, 2H), 4.05 (d, J=7.11 Hz, 1H), 3.97-3.88 (m, 2H), 3.79-3.71 (m, 1H), 3.65-3.56 (m, 2H), 2.79-2.71 (m, 1H), 2.30-1.35 (m, 5H), 1.35-0.90 (m, 9H), 0.87 (td, J=9.48, 8.02, 8.02 Hz, 6H).

(4-Pentylbicyclo[2.2.2]octan-1-yl)methyl ((S)-1-(((S)-1-hydroxy-3-((S)-2-oxopyrrolidin-3-yl)propan-2-yl)amino)-4-methyl-1-oxopentan-2-yl)carbamate (1i). Yield (3 3%). ¹H NMR (400 MHz, DMSO-d₆) δ 7.63-7.45 (m, 1H), 7.09-6.97 (m, 2H), 6.94-4.71 (m, 2H), 4.69-4.57 (m, 5H), 2.90 (s, 1H), 2.78-2.70 (m, 1H), 1.95-1.87 (m, 5H), 1.32 (d, J=7.74 Hz, 6H), 1.23-1.15 (m, 19H), 0.86 (dd, J=13.86, 6.91 Hz, 6H).

(4-Pentylbicyclo[2.2.2]octan-1-yl)methyl-d2 ((S)-1-(((S)-1-hydroxy-3-((S)-2-oxopyrrolidin-3-yl)propan-2-yl)amino)-4-methyl-1-oxopentan-2-yl)carbamate (1j). Yield (20%). ¹H NMR (400 MHz, DMSO-d₆) δ 7.62-7.47 (m, 1H), 7.10-6.97 (m, 2H), 6.97-4.75 (m, 2H), 4.69-4.59 (m, 3H), 2.92 (s, 1H), 2.78-2.76 (m, 1H), 1.95-1.87 (m, 5H), 1.39 (d, J=7.74 Hz, 6H), 1.23-1.17 (m, 19H), 0.86 (dd, J=13.86, 6.91 Hz, 6H).

((3S,5S,7S)-Adamantan-1-yl)methyl ((S)-1-(((S)-1-hydroxy-3-((S)-2-oxopyrrolidin-3-yl)propan-2-yl)amino)-4-methyl-1-oxopentan-2-yl)carbamate (1k). Yield (7 5%). ¹HNMR (400 MHz, CDCl₃) δ 7.75 (d, J=7.1 Hz, 1H), 6.15 (s, 1H), 5.27 (d, J=8.1 Hz, 1H), 4.23-4.18 (m, 1H), 4.03-3.96 (m, 1H), 3.74-3.57 (m, 4H), 3.39-3.31 (m, 2H), 2.49-2.34 (m, 2H), 1.99-1.95 (m, 4H), 1.88-1.79 (m, 1H), 1.76-1.60 (m, 9H), 1.58-1.46 (m, 7H), 0.96 (dd, J=6.4, 2.4 Hz, 6H).

((3S,5S,7S)-Adamantan-1-yl)methyl-d2 ((S)-1-(((S)-1-hydroxy-3-((S)-2-oxopyrrolidin-3-yl)propan-2-yl)amino)-4-methyl-1-oxopentan-2-yl)carbamate (1i). Yield (26%). ¹H NMR (400 MHz, cdcl₃) δ 7.73 (d, J=7.3 Hz, 1H), 6.19 (s, 1H), 5.26 (d, J=8.1 Hz, 1H), 4.22-4.18 (m, 1H), 4.03-3.96 (m, 1H), 3.67-3.54 (m, 2H), 3.39-3.31 (m, 2H), 2.49-2.33 (m, 2H), 2.06-2.02 (m, 1H), 1.99-1.95 (m, 3H), 1.88-1.79 (m, 2H), 1.78-1.60 (m, 9H), 1.57-1.48 (m, 6H), 0.96 (dd, J=6.4, 2.1 Hz, 6H).

2-((3S,5S,7S)-Adamantan-1-yl)ethyl ((S)-1-(((S)-1-hydroxy-3-((S)-2-oxopyrrolidin-3-yl)propan-2-yl)amino)-4-methyl-1-oxopentan-2-yl)carbamate (1m). Yield (65%). ¹H NMR (400 MHz, CDCl₃) δ 7.73 (d, J=7.0 Hz, 1H), 6.24 (s, 1H), 5.28 (d, J=8.3 Hz, 1H), 4.25-4.20 (m, 1H), 4.17-4.07 (m, 2H), 4.04-3.96 (m, 1H), 3.70-3.44 (m, 2H), 3.39-3.31 (m, 2H), 2.52-2.34 (m, 2H), 2.09-1.97 (m, 1H), 1.97-1.91 (m, 3H), 1.90-1.76 (m, 1H), 1.74-1.57 (m, 9H), 1.55-1.45 (m, 7H), 1.40 (t, J=7.5 Hz, 2H), 0.95 (d, J=6.4 Hz, 6H).

((1R,3R,5R,7S)-3-Hydroxyadamantan-1-yl)methyl ((S)-1-(((S)-1-hydroxy-3-((S)-2-oxopyrrolidin-3-yl)propan-2-yl)amino)-4-methyl-1-oxopentan-2-yl)carbamate (In). Yield (8%). 1H NMR (400 MHz, CDCl₃) δ 7.69 (d, J=7.3 Hz, 1H), 6.34 (s, 1H), 5.45 (d, J=8.0 Hz, 1H), 4.20-4.16 (m, 1H), 4.05-3.87 (m, 1H), 3.74-3.54 (m, 4H), 3.37-3.28 (m, 2H), 2.45-2.40 (m, 2H), 2.21 (s, 2H), 2.11-1.94 (m, 1H), 1.88-1.79 (m, 1H), 1.79-1.59 (m, 8H), 1.59-1.54 (m, 2H), 1.54-1.47 (m, 3H), 1.47-1.32 (m, 3H), 0.99-0.88 (m, 6H).

((1r,3R,5R,7S)-3-Hydroxyadamantan-1-yl)methyl-d2 ((S)-1-(((S)-1-hydroxy-3-((S)-2-oxopyrrolidin-3-yl)propan-2-yl)amino)-4-methyl-1-oxopentan-2-yl)carbamate (1o). Yield (7%). ¹H NMR (400 MHz, CDCl₃) δ 7.70 (d, J=7.1 Hz, 1H), 6.26 (s, 1H), 5.41 (d, J=7.9 Hz, 1H), 4.23-4.14 (m, 1H), 4.03-3.96 (m, 1H), 3.68-3.54 (m, 2H), 3.39-3.32 (m, 2H), 2.43 (s, 2H), 2.27-2.19 (m, 2H), 2.11-1.91 (m, 1H), 1.88-1.80 (m, 1H), 1.77-1.60 (m, 8H), 1.60-1.54 (m, 1H), 1.54-1.47 (m, 4H), 1.43-1.38 (m, 3H), 0.99-0.88 (m, 6H).

Preparation of compounds 2(a-o). General procedure. To a solution of dipeptidyl alcohol 1 (1 eq) in anhydrous dichloromethane (300 mL/g dipeptidyl alcohol) kept at 0-5° C. under a N₂ atmosphere was added DMP reagent (3.0 eq) and the reaction mixture was stirred for 3 h at 15-20° C. The organic phase was washed with 10% aq Na₂S₂O₃ (2×100 mL/g dipeptidyl alcohol), followed by saturated aqueous NaHCO₃(2×100 mL/g dipeptidyl alcohol), distilled water (2×100 mL/g dipeptidyl alcohol), and brine (100 mL/g dipeptidyl alcohol). The organic phase was dried over anhydrous Na₂SO₄, filtered and concentrated in vacuo. The resulting crude product was purified by flash chromatography (hexane/ethyl acetate) to yield aldehyde 2 as a white solid.

((1R,3s,5S)-Bicyclo[3.3.1]nonan-3-yl)methyl ((S)-4-methyl-1-oxo-1-(((S)-1-oxo-3-((S)-2-oxopyrrolidin-3-yl)propan-2-yl)amino)pentan-2-yl)carbamate (2a). Yield (86%). ¹H NMR (400 MHz, CDCl₃) δ 9.50 (s, 1H), 8.31 (s, 1H), 5.21 (d, J=8.7 Hz, 1H), 4.38-4.28 (m, 2H), 3.96-3.87 (m, 2H), 3.40-3.31 (m, 2H), 2.54-2.36 (m, 2H), 2.08-1.99 (m, 3H), 1.99-1.81 (m, 5H), 1.76-1.63 (m, 6H), 1.60-1.49 (m, 1H), 1.46-1.29 (m, 4H), 1.15-1.05 (m, 1H), 0.97 (d, J=6.3 Hz, 6H), 0.88 (d, J=13.4 Hz, 2H). HRMS m/z: [M+H]⁺ Calculated for C₂₄H₄₀N₃O₅: 450.2968, Found: 450.2958, m/z: [M+Na]⁺ Calculated for C₂₄H₃₉N₃NaO₅: 472.2788, Found: 472.2776.

((1R,3s,5S)-Bicyclo[3.3.1]nonan-3-yl)methyl-d2 ((S)-4-methyl-1-oxo-1-(((S)-1-oxo-3-((S)-2-oxopyrrolidin-3-yl)propan-2-yl)amino)pentan-2-yl)carbamate (2b). Yield (85%). ¹H NMR (400 MHz, CDCl₃) δ 9.50 (s, 1H), 8.30 (s, 1H), 6.16 (s, 1H), 5.24 (d, J=8.6 Hz, 1H), 4.38-4.29 (m, 2H), 3.41-3.30 (m, 2H), 2.52-2.34 (m, 2H), 2.12-1.99 (m, 3H), 1.98-1.78 (m, 4H), 1.78-1.62 (m, 4H), 1.61-1.51 (m, 1H), 1.41-1.30 (m, 6H), 1.13-1.06 (m, 1H), 0.97 (d, J=6.3 Hz, 6H), 0.96-0.83 (m, 2H). HRMS m/z: [M+Na]⁺ Calculated for C₂₄H₃₇D₂N₃NaO₅: 474.2913, Found: 474.2897.

((1S,5R)-Bicyclo[3.3.1]non-6-en-3-yl)methyl ((S)-4-methyl-1-oxo-1-(((S)-1-oxo-3-((S)-2-oxopyrrolidin-3-yl)propan-2-yl)amino)pentan-2-yl)carbamate (2c). Yield (5 3%). ¹HNMR (400 MHz, CDCl₃) δ 9.49 (s, 1H), 8.27 (s, 1H), 6.04 (s, 1H), 5.86 (t, J=7.1 Hz, 1H), 5.63-5.55 (m, 1H), 5.18 (d, J=8.5 Hz, 1H), 4.38-4.27 (m, 2H), 4.10-4.03 (m, 1H), 4.03-3.94 (m, 1H), 3.41-3.31 (m, 2H), 2.52-2.37 (m, 1H), 2.28 (s, 2H), 2.18-2.08 (m, 1H), 2.01-1.92 (m, 3H), 1.92-1.83 (m, 3H), 1.83-1.71 (m, 1H), 1.71-1.66 (m, 4H), 1.62-1.49 (m, 3H), 1.49-1.36 (m, 1H), 0.97 (d, J=6.4 Hz, 6H). HRMS m/z: [M+H]⁺ Calculated for C₂₄H₃₈N₃O₅: 448.2811, Found: 448.2810, m/z: [M+Na]⁺ Calculated for C₂₄H₃₇N₃NaO₅: 470.2631, Found: 470.2628.

((1S,3S,5R)-Bicyclo[3.3.1]non-6-en-3-yl)methyl-d2 ((S)-4-methyl-1-oxo-1-(((S)-1-oxo-3-((S)-2-oxopyrrolidin-3-yl)propan-2-yl)amino)pentan-2-yl)carbamate (2d). Yield (88%). ¹H NMR (400 MHz, CDCl₃) δ 9.49 (s, 1H), 8.28 (d, J=6.1 Hz, 1H), 6.33 (s, 1H), 5.85 (t, J=8.0 Hz, 1H), 5.58 (d, J=9.9 Hz, 1H), 5.24 (d, J=8.7 Hz, 1H), 4.38-4.29 (m, 2H), 3.43-3.30 (m, 2H), 2.54-2.32 (m, 2H), 2.31-2.26 (m, 1H), 2.20-2.08 (m, 1H), 2.08-1.62 (m, 9H), 1.62-1.46 (m, 4H), 1.45-1.38 (m, 2H), 0.97 (d, J=6.1 Hz, 6H). HRMS m/z: [M+Na]⁺ Calculated for C₂₄H₃₅D₂N₃NaO₅: 472.2757, Found: 472.2743.

Bicyclo[2.2.1]hept-5-en-2-ylmethyl ((S)-4-methyl-1-oxo-1-(((S)-1-oxo-3-((S)-2-oxopyrrolidin-3-yl)propan-2-yl)amino)pentan-2-yl)carbamate (2e). Yield (84%). ¹H NMR (400 MHz, CDCl₃) δ 9.50 (s, 1H), 8.30 (d, J=6.7 Hz, 1H), 6.39 (s, 1H), 6.14 (dd, J=5.8, 3.1 Hz, 1H), 5.93 (dd, J=5.7, 2.9 Hz, 1H), 5.27 (s, 1H), 4.38-4.31 (m, 2H), 3.89-3.80 (m, 1H), 3.69-3.60 (m, 1H), 3.43-3.30 (m, 2H), 2.89-2.78 (m, 1H), 2.56-2.31 (m, 3H), 2.11-1.96 (m, 1H), 1.96-1.90 (m, 1H), 1.90-1.78 (m, 2H), 1.78-1.63 (m, 2H), 1.63-1.49 (m, 1H), 1.48-1.33 (m, 1H), 1.29-1.18 (m, 1H), 1.19-1.10 (m, 1H), 0.97 (d, J=6.3 Hz, 6H), 0.58-0.50 (m, 1H). HRMS m/z: [M+Na]⁺ Calculated for C₂₂H₃₃N₃NaO₅: 442.2318, Found: 442.2310.

Bicyclo[2.2.1]heptan-2-ylmethyl ((S)-4-methyl-1-oxo-1-(((S)-1-oxo-3-((S)-2-oxopyrrolidin-3-yl)propan-2-yl)amino)pentan-2-yl)carbamate (2f). Yield (88%). ¹H NMR (400 MHz, CDCl₃) δ 9.50 (s, 1H), 8.29 (s, 1H), 6.34 (s, 1H), 5.26 (s, 1H), 4.39-4.29 (m, 2H), 4.14-4.03 (m, 1H), 3.97-3.86 (m, 1H), 3.84-3.72 (m, 1H), 3.43-3.30 (m, 2H), 2.54-2.34 (m, 2H), 2.29-2.15 (m, 2H), 2.16-2.08 (m, 1H), 2.08-1.77 (m, 1H), 1.77-1.63 (m, 3H), 1.59-1.43 (m, 2H), 1.41-1.23 (m, 4H), 1.22-1.02 (m, 2H), 0.97 (d, J=6.3 Hz, 6H), 0.67 (ddd, J=12.5, 5.4, 2.3 Hz, 1H). IRMS m/z: [M+Na]⁺ Calculated for C₂₂H₃₅N₃NaO₅: 444.2475, Found: 444.2467.

tert-Butyl (1R,3s,5S)-3-((((S)-4-methyl-1-oxo-1-(((S)-1-oxo-3-((S)-2-oxopyrrolidin-3-yl)propan-2-yl)amino)pentan-2-yl)carbamoyl)oxy)-8-azabicyclo[3.2.1]octane-8-carboxylate (2g). Yield (90%). ¹H NMR (400 MHz, CDCl₃) δ 9.49 (s, 1H), 8.43 (d, J=5.6 Hz, 1H), 6.07 (s, 1H), 5.20 (d, J=8.6 Hz, 1H), 4.96 (s, 1H), 4.35-4.28 (m, 2H), 4.25-4.09 (m, 2H), 3.41-3.33 (m, 2H), 2.53-2.36 (m, 2H), 2.23-1.94 (m, 8H), 1.94-1.82 (m, 1H), 1.81-1.64 (m, 4H), 1.61-1.52 (m, 1H), 1.46 (s, 9H), 0.98 (d, J=5.0 Hz, 6H). HRMS m/z: [M+H]⁺ Calculated for C₂₆H₄₃N₄O₇: 523.3131, Found: 523.3116. HRMS m/z: [M+Na]⁺ Calculated for C₂₆H₄₂N₄NaO₇: 545.2951, Found: 545.2938.

Benzyl (1R,3s,5S)-3-((((S)-4-methyl-1-oxo-1-(((S)-1-oxo-3-((S)-2-oxopyrrolidin-3-yl)propan-2-yl)amino)pentan-2-yl)carbamoyl)oxy)-8-azabicyclo[3.2.1]octane-8-carboxylate (2h). Yield (66%). ¹H NMR (400 MHz, DMSO-d₆) δ 9.39 (s, 1H), 8.55-8.35 (m, 1H), 8.11-8.02 (m, 1H), 7.64 (s, 1H), 7.45-7.19 (m, 5H), 5.75 (s, 1H), 5.09 (d, J=10.47 Hz, 2H), 4.83-4.71 (m, 2H), 4.21 (s, 2H), 3.73-3.57 (m, 2H), 3.24-3.00 (m, 2H), 2.34-1.79 (m, 3H), 1.79-1.34 (m, 2H), 1.28-1.12 (m, 9H), 0.98-0.77 (m, 6H). HRMS m/z: [M+H]⁺ Calculated for C₂₉H₄₁N₄O₇: 557.2970, Found: 557.2962. HRMS m/z: [M+Na]⁺ Calculated for C₂₉H₄₀N₄NaO₇: 579.2789, Found: 579.2773.

(4-Pentylbicyclo[2.2.2]octan-1-yl)methyl ((S)-4-methyl-1-oxo-1-(((S)-1-oxo-3-((S)-2-oxopyrrolidin-3-yl)propan-2-yl)amino)pentan-2-yl)carbamate (2i). Yield (59%). ¹H NMR (400 MHz, DMSO-d₆) δ 9.49 (s, 1H), 7.66-7.58 (m, 1H), 7.54-7.44 (m, 2H), 5.72-5.64 (m, 2H), 3.71-3.53 (m, 5H), 3.21-3.13 (m, 1H), 2.93-2.85 (m, 1H), 2.77-2.69 (m, 1H), 2.30-2.22 (m, 1H), 1.91 (s, 1H), 1.63-1.55 (m, 5H), 1.42-0.96 (m, 20H), 0.87 (td, J=19.27, 6.96, 6.96 Hz, 6H). HRMS m/z: [M+Na]⁺ Calculated for C₂₈H₄₇N₃NaO₅: 528.3414, Found: 528.3391.

(4-Pentylbicyclo[2.2.2]octan-1-yl)methyl-d2 ((S)-4-methyl-1-oxo-1-(((S)-1-oxo-3-((S)-2-oxopyrrolidin-3-yl)propan-2-yl)amino)pentan-2-yl)carbamate (2j). Yield (49%). ¹H NMR (400 MHz, DMSO-d₆) δ 9.49 (s, 1H), 7.66-7.58 (m, 1H), 7.54-7.44 (m, 2H), 5.72-5.64 (m, 2H), 3.71-3.63 (m, 3H), 3.31 (s, 1H), 2.93-2.85 (m, 1H), 2.85-2.69 (m, 1H), 2.30-2.22 (m, 1H), 1.91 (s, 1H), 1.42-0.96 (m, 25H), 0.87 (td, J=19.21, 6.96, 6.96 Hz, 6H). HRMS m/z: [M+Na]⁺ Calculated for C₂₈H₄₅D₂N₃NaO₅: 530.3539, Found: 530.3536.

((3S,5S,7S)-Adamantan-1-yl)methyl((S)-4-methyl-1-oxo-1-(((S)-1-oxo-3-((S)-2-oxopyrrolidin-3-yl)propan-2-yl)amino)pentan-2-yl)carbamate (2k). Yield (90%). ¹H NMR (400 MHz, CDCl₃) δ 9.50 (s, 1H), 8.31 (d, J=5.8 Hz, 1H), 6.36 (s, 1H), 5.27 (s, 1H), 4.39-4.29 (m, 2H), 3.66 (s, 2H), 3.41-3.30 (m, 2H), 2.55-2.34 (m, 2H), 2.08-1.92 (m, 3H), 1.92-1.78 (m, 2H), 1.78-1.60 (m, 9H), 1.52 (d, J=2.9 Hz, 7H), 1.00-0.94 (m, 6H). HRMS m/z: [M+Na]⁺ Calculated for C₂₅H₃₉N₃NaO₅: 484.2788, Found: 484.2780.

((3S,5S,7S)-Adamantan-1-yl)methyl-d2 ((S)-4-methyl-1-oxo-1-(((S)-1-oxo-3-((S)-2-oxopyrrolidin-3-yl)propan-2-yl)amino)pentan-2-yl)carbamate (2). Yield (8 8%). ¹H NMR (400 MHz, cdcl₃) δ 9.49 (s, 1H), 8.32 (d, J=5.9 Hz, 1H), 6.13 (d, J=8.1 Hz, 1H), 5.23 (s, 1H), 4.68-4.58 (m, 1H), 4.38-4.25 (m, 1H), 3.41-3.27 (m, 2H), 2.52-2.34 (m, 2H), 2.06-2.01 (m, 2H), 2.00-1.89 (m, 3H), 1.88-1.78 (m, 4H), 1.78-1.55 (m, 9H), 1.52 (d, J=2.9 Hz, 3H), 1.00-0.91 (m, 6H). HRMS m/z: [M+Na]⁺ Calculated for C₂₅H₃₇D₂N₃NaO₅: 486.2913, Found: 486.2910.

2-((3S,5S,7S)-Adamantan-1-yl)ethyl ((S)-4-methyl-1-oxo-1-(((S)-1-oxo-3-((S)-2-oxopyrrolidin-3-yl)propan-2-yl)amino)pentan-2-yl)carbamate (2m). Yield (83%). ¹H NMR (400 MHz, CDCl₃) δ 9.50 (s, 1H), 8.30 (d, J=5.9 Hz, 1H), 6.09 (d, J=11.7 Hz, 1H), 5.19 (d, J=8.7 Hz, 1H), 4.38-4.28 (m, 2H), 4.26-4.05 (m, 2H), 3.41-3.33 (m, 2H), 2.54-2.35 (m, 2H), 2.01-1.91 (m, 4H), 1.91-1.79 (m, 1H), 1.74-1.58 (m, 9H), 1.51 (d, J=2.9 Hz, 6H), 1.41 (t, J=7.4 Hz, 2H), 1.28-1.23 (m, 1H), 0.97 (d, J=6.4 Hz, 6H). HRMS m/z: [M+H]⁺ Calculated for C₂₆H₄₂N₃O₅: 476.3124, Found: 476.3124. HRMS m/z: [M+Na]⁺ Calculated for C₂₆H₄₁N₃NaO₅: 498.2944, Found: 498.2938.

((1r,3R,5R,7S)-3-Hydroxyadamantan-1-yl)methyl ((S)-4-methyl-1-oxo-1-(((S)-1-oxo-3-((S)-2-oxopyrrolidin-3-yl)propan-2-yl)amino)pentan-2-yl)carbamate (2n). Yield (47%). ¹H NMR (400 MHz, CDCl₃) δ 9.49 (s, 1H), 8.28 (d, J=23.9 Hz, 1H), 6.19 (s, 1H), 5.19 (s, 1H), 4.34-4.03 (m, 2H), 4.02-3.61 (m, 2H), 2.89-2.58 (m, 2H), 2.58-2.28 (m, 2H), 2.28-2.13 (m, 3H), 2.09-1.76 (m, 2H), 1.78-1.60 (m, 6H), 1.57-1.31 (m, 9H), 1.01-0.91 (m, 6H). HRMS m/z: [M+Na]⁺ Calculated for C₂₅H₃₉N₃NaO₆: 500.2737, Found: 500.2739.

((1r,3R,5R,7S)-3-Hydroxyadamantan-1-yl)methyl-d2 ((S)-4-methyl-1-oxo-1-(((S)-1-oxo-3-((S)-2-oxopyrrolidin-3-yl)propan-2-yl)amino)pentan-2-yl)carbamate (2o). Yield (44%). ¹H NMR (400 MHz, CDCl₃) δ 9.48 (s, 1H), 8.28 (d, J=6.5 Hz, 1H), 6.39 (s, 1H), 5.12 (s, 1H), 4.40-4.09 (m, 2H), 2.89-2.59 (m, 2H), 2.56-2.30 (m, 2H), 2.30-2.20 (m, 3H), 2.18-1.77 (m, 2H), 1.77-1.61 (m, 6H), 1.59-1.39 (m, 9H), 1.01-0.89 (m, 6H). HRMS m/z: [M+Na]⁺ Calculated for C₂₅H₃₇D₂N₃NaO₆: 502.2862, Found: 502.2860.

Preparation of compounds 3(a-o). General procedure. To a solution of dipeptidyl aldehyde 2 (1 eq) in ethyl acetate (10 mL/g of dipeptidyl aldehyde) was added absolute ethanol (5 mL/g of dipeptidyl aldehyde) with stirring, followed by a solution of sodium bisulfite (1 eq) in water (1 mL/g of dipeptidyl aldehyde). The reaction mixture was stirred for 3 h at 50° C. The reaction mixture was allowed to cool to room temperature and then vacuum filtered. The solid was thoroughly washed with absolute ethanol and the filtrate was dried over anhydrous sodium sulfate, filtered, and concentrated to yield a white solid. The white solid was stirred with dry ethyl ether (3×10 mL/g of dipeptidyl aldehyde), followed by careful removal of the solvent using a pipette and dried using a vacuum pump for 2 h to yield dipeptidyl bisulfite adduct 3 as a white solid.

Sodium (2S)-2-((2S)-2-(((((1R,5S)-bicyclo[3.3.1]nonan-3-yl)methoxy)carbonyl)amino)-4-methylpentanamido)-1-hydroxy-3-((S)-2-oxopyrrolidin-3-yl)propane-1-sulfonate (3a). Yield (66%). ¹H NMR (400 MHz, DMSO-d₆) δ 7.61 (d, J=9.0 Hz, 1H), 7.57-7.44 (m, 1H), 7.28-7.08 (m, 1H), 5.62 (d, J=6.1 Hz, 1H), 5.47 (d, J=5.9 Hz, 1H), 4.12-3.91 (m, 2H), 3.91-3.73 (m, 2H), 3.16-3.00 (m, 2H), 2.24-2.07 (m, 2H), 2.06-1.95 (m, 2H), 1.83 (dt, J=17.2, 7.2 Hz, 4H), 1.74-1.51 (m, 5H), 1.51-1.38 (m, 2H), 1.35-1.15 (m, 6H), 1.14-1.00 (m, 2H), 0.99-0.81 (m, 6H). HRMS m/z: [M+Na]⁺ Calculated for C₂₄H₄₀N₃Na₂O₈S: 576.2332, Found: 576.2329, m/z: [M]⁻ Calculated for C₂₄H₄₀N₃O₈S: 530.2536, Found: 530.2529.

Sodium (2S)-2-((S)-2-(((((1R,3s,5S)-bicyclo[3.3.1]nonan-3-yl)methoxy-d2)carbonyl)amino)-4-methylpentanamido)-1-hydroxy-3-((S)-2-oxopyrrolidin-3-yl)propane-1-sulfonate (3b). Yield (75%). ¹H NMR (400 MHz, DMSO-d₆) δ 7.63-7.43 (m, 2H), 7.20 (dd, J=20.3, 8.2 Hz, 1H), 5.52-5.33 (m, 1H), 4.06-3.78 (m, 2H), 3.20-2.99 (m, 2H), 2.33-2.04 (m, 3H), 2.04-1.93 (m, 2H), 1.94-1.74 (m, 2H), 1.74-1.50 (m, 3H), 1.50-1.38 (m, 2H), 1.38-1.19 (m, 5H), 1.11-0.98 (m, 2H), 0.97-0.80 (m, 10H). HRMS m/z: [M+Na]⁺ Calculated for C₂₄H₃₈D₂N₃Na₂O₈S: 578.2457, Found: 578.2432.

Sodium (2S)-2-((2S)-2-(((((1S,5R)-bicyclo[3.3.1]non-6-en-3-yl)methoxy)carbonyl)amino)-4-methylpentanamido)-1-hydroxy-3-((S)-2-oxopyrrolidin-3-yl)propane-1-sulfonate (3c). Yield (66%). ¹H NMR (400 MHz, DMSO-d₆) δ 7.52 (d, J=9.5 Hz, 1H), 7.47 (d, J=4.5 Hz, 1H), 7.18 (ddd, J=19.5, 8.5, 2.7 Hz, 1H), 5.89-5.79 (m, 1H), 5.58-5.52 (m, 1H), 3.97 (h, J=8.1 Hz, 1H), 3.90-3.70 (m, 1H), 3.27-3.00 (m, 2H), 2.35-2.19 (m, 3H), 2.19-2.02 (m, 3H), 2.02-1.68 (m, 6H), 1.68-1.49 (m, 4H), 1.49-1.25 (m, 6H), 1.13-1.04 (m, 1H), 0.91-0.79 (m, 6H). HRMS m/z: [M+Na]⁺ Calculated for C₂₄H₃₈N₃Na2O₈S: 574.2175, Found: 574.2163, m/z: [M]⁻ Calculated for C₂₄H₃₈N₃O₈S: 528.2379, Found: 528.2367.

Sodium (2S)-2-((S)-2-(((((1S,3S,5R)-bicyclo[3.3.1]non-6-en-3-yl)methoxy-d2)carbonyl) amino)-4-methylpentanamido)-1-hydroxy-3-((S)-2-oxopyrrolidin-3-yl)propane-1-sulfonate (3d). Yield (53%). ¹H NMR (400 MHz, DMSO-d₆) δ 7.62-7.39 (m, 2H), 7.18 (dd, J=24.9, 8.5 Hz, 1H), 5.89-5.80 (m, 1H), 5.59-5.52 (m, 1H), 5.43-5.26 (m, 1H), 4.06-3.67 (m, 2H), 3.22-2.98 (m, 2H), 2.37-2.03 (m, 5H), 2.03-1.64 (m, 5H), 1.64-1.52 (m, 3H), 1.52-1.29 (m, 5H), 1.13-1.02 (m, 1H), 0.89-0.80 (m, 6H). HRMS m/z: [M+Na]⁺ Calculated for C₂₄H₃₆D₂N₃Na₂O₈S: 576.2301, Found: 576.2275.

Sodium (2S)-2-((2S)-2-(((bicyclo[2.2.1]hept-5-en-2-ylmethoxy)carbonyl)amino)-4-methylpentanamido)-1-hydroxy-3-((S)-2-oxopyrrolidin-3-yl)propane-1-sulfonate (3e). Yield (56%). ¹H NMR (400 MHz, DMSO-d₆) δ 7.68-7.37 (m, 2H), 7.32-7.17 (m, 1H), 6.20-6.02 (m, 1H), 6.02-5.86 (m, 1H), 5.59-5.30 (m, 1H), 4.11-3.79 (m, 2H), 3.79-3.41 (m, 2H), 3.21-2.98 (m, 2H), 2.87-2.66 (m, 2H), 2.39-2.04 (m, 3H), 2.04-1.85 (m, 1H), 1.85-1.70 (m, 1H), 1.70-1.51 (m, 2H), 1.50-1.36 (m, 2H), 1.28 (dd, J=39.4, 7.9 Hz, 2H), 1.18-1.00 (m, 1H), 0.93-0.79 (m, 6H), 0.47 (d, J=11.6 Hz, 1H). HRMS m/z: [M+Na]⁺ Calculated for C₂₂H₃₄N₃Na₂O₈S: 546.1862, Found: 546.1842.

Sodium (2S)-2-((2S)-2-(((bicyclo[2.2.1]heptan-2-ylmethoxy)carbonyl)amino)-4-methyl pentanamido)-1-hydroxy-3-((S)-2-oxopyrrolidin-3-yl)propane-1-sulfonate (3f). Yield (66%). ¹H NMR (400 MHz, DMSO-d₆) δ 7.61-7.39 (m, 2H), 7.32-7.08 (m, 1H), 5.41 (dd, J=55.1, 5.9 Hz, 1H), 4.07-3.55 (m, 2H), 3.18-2.99 (m, 2H), 2.28-2.04 (m, 6H), 2.04-1.81 (m, 1H), 1.81-1.53 (m, 3H), 1.53-1.36 (m, 5H), 1.36-1.18 (m, 3H), 1.18-0.91 (m, 2H), 0.90-0.75 (m, 6H), 0.70-0.59 (m, 1H). HRMS m/z: [M+Na]⁺ Calculated for C₂₂H₃₆N₃Na₂O₈S: 548.2019, Found: 548.1999.

Sodium (2S)-2-((S)-2-(((((1R,3s,5S)-8-(tert-butoxycarbonyl)-8-azabicyclo[3.2.1]octan-3-yl)oxy)carbonyl)amino)-4-methylpentanamido)-1-hydroxy-3-((S)-2-oxopyrrolidin-3-yl)propane-1-sulfonate (3g). Yield (52%). ¹H NMR (400 MHz, DMSO-d₆) δ 7.64-7.51 (m, 1H), 7.46 (d, J=3.8 Hz, 1H), 7.30 (dd, J=19.2, 8.3 Hz, 1H), 5.55-5.31 (m, 1H), 4.76 (s, 1H), 4.02-3.78 (m, 4H), 3.22-2.98 (m, 2H), 2.27-1.68 (m, 9H), 1.66-1.57 (m, 4H), 1.51-1.43 (m, 2H), 1.40 (s, 9H), 1.13-1.02 (m, 1H), 0.91-0.81 (m, 6H). HRMS m/z: [M+Na]⁺ Calculated for C₂₆H₄₃N₄Na₂O₁₀S: 649.2496, Found: 649.2500.

Sodium (2S)-2-((S)-2-(((((1R,3s,5S)-8-((benzyloxy)carbonyl)-8-azabicyclo[3.2.1]octan-3-yl)oxy)carbonyl)amino)-4-methylpentanamido)-1-hydroxy-3-((S)-2-oxopyrrolidin-3-yl)propane-1-sulfonate (3h). Yield (53%). 1H NMR (400 MHz, DMSO-d₆) δ 7.37 (t, J=4.74, 4.74 Hz, 8H), 5.09 (d, J=11.06 Hz, 2H), 4.29-4.13 (m, 5H), 3.96-3.82 (m, 1H), 3.79-3.69 (m, 1H), 3.17-3.09 (m, 1H), 3.06-2.98 (m, 1H), 2.05-1.81 (m, 5H), 1.74-1.63 (m, 3H), 1.58-1.49 (m, 4H), 1.47-1.33 (m, 2H), 1.08 (dd, J=13.72, 6.78 Hz, 1H), 0.84 (ddd, J=10.35, 8.11, 4.59 Hz, 6H). HRMS m/z: [M+Na]⁺ Calculated for C₂₉H₄₁N₄Na₂O₁₀S: 683.2339, Found: 683.2317.

Sodium (2S)-1-hydroxy-2-((S)-4-methyl-2-((((4-pentylbicyclo[2.2.2]octan-1-yl)methoxy) carbonyl)amino)pentanamido)-3-((S)-2-oxopyrrolidin-3-yl)propane-1-sulfonate (3i). Yield (57%). 1H NMR (400 MHz, DMSO-d₆) δ 7.51-7.40 (m, 3H), 4.00-3.53 (m, 5H), 3.49-3.40 (m, 1H), 3.16-2.98 (m, 4H), 1.94-1.85 (m, 3H), 1.65-0.96 (m, 25H), 0.85 (dd, J=12.73, 5.76 Hz, 6H). HRMS m/z: [M+Na]⁺ Calculated for C₂₈H₄₈N₃Na₂O₈S: 632.2958, Found: 632.2932. HRMS m/z: [M]⁻ Calculated for C₂₈H₄₈N₃O₈S: 586.3168, Found: 586.3163.

Sodium (2S)-1-hydroxy-2-((S)-4-methyl-2-((((4-pentylbicyclo[2.2.2]octan-1-yl)methoxy-d2)carbonyl)amino)pentanamido)-3-((S)-2-oxopyrrolidin-3-yl)propane-1-sulfonate (3j). Yield (50%). 1H NMR (400 MHz, DMSO-d₆) δ 7.51-7.40 (m, 3H), 4.00-3.53 (m, 3H), 3.40-3.26 (m, 1H), 3.16-2.90 (m, 3H), 2.56-2.44 (m, 1H), 1.95-1.85 (m, 3H), 1.65-0.96 (m, 25H), 0.85 (dd, J=12.73, 5.76 Hz, 6H). HRMS m/z: [M+Na]⁺ Calculated for C₂₅H₄₆D₂N₃Na₂O₈S: 634.3083, Found: 634.3071. HRMS m/z: [M]⁻ Calculated for C₂₈H₄₆D₂N₃O₈S: 588.3287, Found: 588.3399.

Sodium (2S)-2-((S)-2-(((((3S,5S,7S)-adamantan-1-yl)methoxy)carbonyl)amino)-4-methylpentanamido)-1-hydroxy-3-((S)-2-oxopyrrolidin-3-yl)propane-1-sulfonate (3k). Yield (52%). ¹H NMR (400 MHz, DMSO-d₆) δ 7.66-7.41 (m, 2H), 7.25-7.10 (m, 1H), 5.40 (dd, J=47.1, 6.0 Hz, 1H), 4.10-3.70 (m, 2H), 3.69-3.47 (m, 2H), 3.18-3.00 (m, 2H), 2.24-2.03 (m, 2H), 2.03-1.83 (m, 4H), 1.83-1.55 (m, 8H), 1.55-1.33 (m, 6H), 1.13-1.02 (m, 1H), 0.93-0.80 (m, 6H). HRMS m/z: [M+Na]⁺ Calculated for C₂₅H₄₀N₃Na₂O₈S: 588.2332, Found: 588.2310.

Sodium (2S)-2-((S)-2-(((((3S,5S,7S)-adamantan-1-yl)methoxy-d2)carbonyl)amino)-4-methylpentanamido)-1-hydroxy-3-((S)-2-oxopyrrolidin-3-yl)propane-1-sulfonate (3l). Yield (39%). ¹H NMR (400 MHz, DMSO-d₆) δ 7.63-7.50 (m, 1H), 7.47-7.36 (m, 1H), 7.38-7.28 (m, 1H), 5.41 (dd, J=36.5, 6.0 Hz, 1H), 4.27-4.19 (m, 1H), 3.99-3.94 (m, 1H), 3.49-3.34 (m, 1H), 3.21-3.01 (m, 2H), 2.25-1.99 (m, 2H), 1.99-1.90 (m, 3H), 1.89-1.73 (m, 3H), 1.72-1.52 (m, 9H), 1.48 (s, 5H), 0.90-0.76 (m, 6H). HRMS m/z: [M+Na]⁺ Calculated for C₂₅H₃₈D₂N₃Na₂O₈S: 590.2457, Found: 590.2447.

Sodium (2S)-2-((S)-2-(((2-((3S,5S,7S)-adamantan-1-yl)ethoxy)carbonyl)amino)-4-methyl pentanamido)-1-hydroxy-3-((S)-2-oxopyrrolidin-3-yl)propane-1-sulfonate (3m). Yield (60%). ¹H NMR (400 MHz, DMSO-d₆) δ 7.62-7.49 (m, 1H), 7.49-7.43 (m, 1H), 7.24-7.07 (m, 1H), 5.40 (dd, J=48.6, 6.1 Hz, 1H), 4.05-3.81 (m, 4H), 3.20-3.00 (m, 2H), 2.26-2.06 (m, 2H), 2.01-1.87 (m, 3H), 1.84-1.72 (m, 1H), 1.70-1.54 (m, 10H), 1.49 (d, J=3.0 Hz, 6H), 1.45-1.38 (m, 1H), 1.38-1.28 (m, 2H), 0.89-0.80 (m, 6H). HRMS m/z: [M+Na]⁺ Calculated for C₂₆H₄₂N₃Na₂O₈S: 602.2488, Found: 602.2480.

Sodium (2S)-1-hydroxy-2-((S)-2-(((((1r,3R,5R,7S)-3-hydroxyadamantan-1-yl)methoxy) carbonyl)amino)-4-methylpentanamido)-3-((S)-2-oxopyrrolidin-3-yl)propane-1-sulfonate (3n). Yield (69%). ¹H NMR (400 MHz, acetone) 6 7.08-7.03 (m, 1H), 7.00-6.95 (m, 1H), 6.45-6.41 (m, 1H), 4.38-3.89 (m, 2H), 3.89-3.53 (m, 2H), 2.96-2.53 (m, 1H), 2.53-2.25 (m, 2H), 2.16 (s, 2H), 1.99-1.70 (m, 4H), 1.70-1.62 (m, 5H), 1.62-1.53 (m, 4H), 1.53-1.35 (m, 5H), 1.34-1.16 (m, 1H), 0.99-0.85 (m, 6H). HRMS m/z: [M+Na]⁺ Calculated for C₂₅H₄₀N₃Na₂O₉S: 604.2282, Found: 604.2273.

Sodium (2S)-1-hydroxy-2-((S)-2-(((((1r,3R,5R,7S)-3-hydroxyadamantan-1-yl)methoxy-d2)carbonyl)amino)-4-methylpentanamido)-3-((S)-2-oxopyrrolidin-3-yl)propane-1-sulfonate (3o). Yield (73%). ¹H NMR (400 MHz, acetone) 6 7.10-7.05 (m, 1H), 6.91-6.87 (m, 1H), 6.43-6.38 (m, 1H), 4.33-4.06 (m, 2H), 3.33-3.19 (m, 2H), 2.50-2.24 (m, 1H), 2.22-2.10 (m, 3H), 2.03-1.71 (m, 2H), 1.70-1.61 (m, 5H), 1.61-1.52 (m, 5H), 1.52-1.38 (m, 6H), 1.01-0.85 (m, 6H). HRMS m/z: [M+Na]⁺ Calculated for C₂₅H₃₈D₂N₃Na₂O₉S: 606.2406, Found: 606.2409.

### Biochemical Studies

Enzyme assays and inhibition studies. Cloning and expression of the 3CL protease of SARS-CoV-2 and FRET enzyme assays. The codon-optimized cDNA of full length of 3CLpro of SARS-CoV-2 (GenBank number MN908947.3) fused with sequences encoding 6 histidine at the N-terminal was synthesized by Integrated DNA (Coralville, IA). The synthesized gene was subcloned into the pET-28a(+) vector. The expression and purification of SARS-CoV-2 3CLpro were conducted following a standard procedure. Briefly, a stock solution of an inhibitor was prepared in DMSO and diluted in assay buffer comprised of 20 mM HEPES buffer, pH 8, containing NaCl (200 mM), EDTA (0.4 mM), glycerol (60%), and 6 mM dithiothreitol (DTT). The SARS-CoV-2 protease was mixed with serial dilutions of inhibitor or with DMSO in 25 μL of assay buffer and incubated at 37° C. for 1 h, followed by the addition of 25 μL of assay buffer containing substrate (FAM-SAVLQ/SG-QXL®520, AnaSpec, Fremont, CA). The substrate was derived from the cleavage sites on the viral polyproteins of SARS-CoV. Fluorescence readings were obtained using an excitation wavelength of 480 nm and an emission wavelength of 520 nm on a fluorescence microplate reader (FLx800; Biotec, Winoosk, VT) 1 h following the addition of substrate. Relative fluorescence units (RFU) were determined by subtracting background values (substrate-containing well without protease) from the raw fluorescence values using established procedures. The dose-dependent FRET inhibition curves were fitted with a variable slope by using GraphPad Prism software (GraphPad, La Jolla, CA) in order to determine the IC₅₀ values of the compounds. The expression and purification of the 3CLpro of MERS-CoV, as well as the FRET enzyme assays were performed using an established procedure.

Cell-based assay for antiviral activity. Representative compounds 2a and 3a were investigated for their antiviral activity against the replication of SARS-CoV-2. Briefly, confluent Vero E6 cells were inoculated with SARS-CoV-2 at 50-100 plaque forming units/well, and medium containing various concentrations of each compound and agar was applied to the cells. After 48-72 hr, plaques in each well were counted. The 50% effective concentration (EC₅₀) values were determined by GraphPad Prism software using a variable slope (GraphPad, La Jolla, CA).

Nonspecific cytotoxic effects/In vitro cytotoxicity. Confluent cells grown in 96-well plates were incubated with various concentrations (1 to 100 μM) of each compound for 72 h. Cell cytotoxicity was measured by a CytoTox 96 nonradioactive cytotoxicity assay kit (Promega, Madison, WI), and the CC₅₀ values were calculated using a variable slope by GraphPad Prism software. The in vitro Safety Index was calculated by dividing the CC₅₀ by the IC₅₀.

### X-Ray Crystallographic Studies

Crystallization and Data Collection. Purified SARS-2 3CL protease (SARS-2 3CLpro) in 100 mM NaCl, 20 mM Tris buffer, pH 8.0, was concentrated to 9.6 mg/mL (0.28 mM) for crystallization screening. All crystallization experiments were set up using an NT8 drop-setting robot (Formulatrix Inc.) and UVXPO MRC (Molecular Dimensions) sitting drop vapor diffusion plates at 18° C. 100 nL of protein and 100 nL crystallization solution were dispensed and equilibrated against 50 μL of the latter. Stock solutions of the inhibitors (100 mM) were prepared in DMSO and the complexes were obtained by mixing 1 μL of the ligand (2 mM) with 49 μL (0.28 mM) of SARS-2 3CLpro and incubating on ice for 1 h. Crystals were obtained in 1-2 days from the following conditions. 2a and 3b: Berkeley screen (Rigaku Reagents) condition C5 (20% (w/v) PEG 4000, 100 mM Tris pH 8.0), 2f: Index HT screen (Hampton Research) condition H6 (20% (w/v) PEG 3350, 200 mM sodium formate), 2k: Proplex HT screen (Molecular Dimensions) condition D7 (15% (w/v) PEG 6000, 100 mM sodium citrate pH 5.5), 3c and 3d: the Berkeley screen (Rigaku Reagents) condition D9 (20% (w/v) PEG 3350, 100 mM Bis-Tris pH 6.5, 100 mM ammonium phosphate dibasic, 5% (v/v) 2-propanol) and 3e: Index HT screen (Hampton Research) condition C₅ (15% (w/v) PEG 3350, 100 mM succinic acid pH 7.0). Samples were transferred to cryoprotectant solutions, prior to plunging in liquid nitrogen, composed of 80% crystallization solution and 20% (v/v) PEG 200 except for 3c and 3d for which 20% (v/v) ethylene glycol was used as the cryoprotectant. X-ray diffraction data were collected at the Advanced Photon Source IMCA-CAT beamline 17-ID except for the data for the complex with 3c which were collected at the National Synchrotron Light Source II (NSLS-II) AMX beamline 17-ID-1.

Structure Solution and Refinement. Intensities were integrated using XDS (X-ray detector software) via Autoproc and the Laue class analysis and data scaling were performed with Aimless. Structure solution was conducted by molecular replacement with Phaser using a previously determined structure of SARS-2 3CLpro (PDB 6XM1K) as the search model. Structure refinement and manual model building were conducted with Phenix and Coot, respectively. Disordered side chains were truncated to the point for which electron density could be observed. Structure validation was conducted with Molprobity and structure analysis/figure preparation were carried out using the CCP4 mg package. Crystallographic data are provided in Table 5. Coordinates and structure factors for the following SARS2 3CLpro complexes with inhibitors were deposited to the Worldwide Protein Databank (wwPDB) with the accession codes: 7LKR (2a), 7LKS (2f), 7LKT (2k), 7LKU (3b), 7LKV (3c), 7LKW (3d) and 7LKX (3e).

TABLE 5
Crystallographic data for SARS-CoV-2 3CLpro inhibitor complexes.

| | Inhibitor PDB Code | | | | | | |
|---|---|---|---|---|---|---|---|
| | 2a | 2f | 2k | 3b | 3c | 3d | 3e |
| | (7LKR) | (7LKS) | (7LKT) | (7LKU) | (7LKV) | (7LKW) | (7LKX) |
| Data Collection | | | | | | | |
| Unit-cell | a = 55.47 | a = 55.19 | a = 55.10 | a = 54.91 | a = 55.59 | a = 54.91 | a = 55.30 |
| parameters | b = 98.62 | b = 98.76 | b = 98.86 | b = 98.39 | b = 98.88 | b = 98.39 | b = 98.75 |
| (Å, °) | c = 58.94 | c = 58.85 | c = 59.42 | c = 58.19 | c = 58.96 | c = 58.19 | c = 59.09 |
| | β = 108.2 | β = 107.6 | β = 107.9 | β = 107.4 | β = 108.3 | β = 107.4 | β = 108.0 |
| Space group | P2₁ | P2₁ | P2₁ | P2₁ | P2₁ | P2₁ | P2₁ |
| Resolution (Å)¹ | 48.69-1.65 | 49.38-1.70 | 49.08-1.50 | 49.19-1.65 | 46.56-1.55 | 49.19-1.65 | 48.83-1.60 |
| | (1.68-1.65) | (1.73-1.70) | (1.53-1.50) | (1.68-1.65) | (1.58-1.55) | (1.68-1.65) | (1.63-1.60) |
| Wavelength (Å) | 1.0000 | 1.0000 | 1.0000 | 1.0000 | 0.9201 | 1.0000 | 1.0000 |
| Temperature (K) | 100 | 100 | 100 | 100 | 100 | 100 | 100 |
| Observed | 248,528 | 429,846 | 336,406 | 246,165 | 299,258 | 246,165 | 277,540 |
| reflections | | | | | | | |
| Unique | 72,217 | 65,020 | 96,612 | 70,811 | 86,929 | 70,811 | 77,631 |
| reflections | | | | | | | |
| <I/σ(I)>¹ | 8.6 (2.4) | 11.4 (1.7) | 10.5 (1.8) | 9.8 (1.9) | 8.9 (1.8) | 9.8 (1.9) | 9.1 (1.8) |
| Completeness | 99.9 (98.4) | 98.4 (100) | 99.9 (99.9) | 99.9 (100) | 99.4 (99.3) | 99.9 (100) | 97.9 (96.4) |
| (%)¹ | | | | | | | |
| Multiplicity¹ | 3.4 (2.7) | 6.6 (7.0) | 3.5 (3.2) | 3.5 (3.5) | 3.4 (3.6) | 3.5 (3.5) | 3.6 (3.7) |
| R_{merge} (%)^{1, 2} | 9.3 (46.0) | 7.9 (141.5) | 6.3 (67.5) | 5.4 (55.3) | 7.2 (69.9) | 5.4 (55.3) | 7.1 (81.4) |
| Rₘₑₐₛ (%)^{1, 4} | 11.0 (57.0) | 8.6 (153.0) | 7.4 (81.5) | 6.4 (65.3) | 8.5 (82.2) | 6.4 (65.3) | 8.4 (95.3) |
| Rₚᵢₘ (%)^{1, 4} | 5.8 (33.1) | 3.3 (57.6) | 3.9 (45.0) | 3.4 (34.3) | 4.5 (42.9) | 3.4 (34.3) | 4.4 (49.2) |
| CC_{1/2}^{1, 5} | 0.989 (0.819) | 0.999 (0.801) | 0.997 (0.573) | 0.998 (0.883) | 0.996 (0.718) | 0.998 (0.883) | 0.996 (0.696) |
| Refinement | | | | | | | |
| Resolution (Å)¹ | 37.01-1.65 | 36.00-1.70 | 37.21-1.50 | 31.66-1.65 | 46.56-1.55 | 45.72-1.70 | 35.99-1.60 |
| Reflections | 68,631/3,538 | 61,732/3,127 | 91,730/4,828 | 67,198/3,470 | 82,504/4,340 | 61,623/3,225 | 73934/3,644 |
| (working/test)¹ | | | | | | | |
| R_{factor}/R_{free} | 18.0/21.9 | 18.4/22.1 | 17.8/20.5 | 17.8/21.1 | 17.8/20.8 | 18.6/22.7 | 18.4/21.2 |
| (%)^{1, 3} | | | | | | | |
| # of atoms | 4,467/128/ | 4,492/120/ | 4,558/132/ | 4,524/128/ | 4,495/128/ | 4,448/128/ | 4,460/120/ |
| (Protein/ | 369 | 282 | 420 | 323 | 442 | 224 | 329 |
| Ligand/Water) | | | | | | | |
| Model Quality | | | | | | | |
| R.m.s deviations | | | | | | | |
| Bond lengths (Å) | 0.009 | 0.010 | 0.010 | 0.008 | 0.009 | 0.010 | 0.007 |
| Bond angles (°) | 0.959 | 1.068 | 1.070 | 0.932 | 1.012 | 1.051 | 0.901 |
| Mean B-factor | | | | | | | |
| (Å²) | | | | | | | |
| All Atoms | 27.0 | 33.4 | 26.0 | 31.6 | 27.5 | 36.0 | 30.7 |
| Protein | 26.3 | 33.0 | 25.2 | 31.0 | 26.5 | 35.5 | 29.8 |
| Ligand | 31.5 | 35.3 | 23.3 | 35.2 | 29.6 | 41.6 | 34.9 |
| Water | 53.2 | 39.1 | 34.6 | 39.3 | 37.0 | 40.6 | 37.3 |
| Coordinate error | 0.20 | 0.21 | 0.17 | 0.21 | 0.21 | 0.19 | 0.21 |
| (maximum | | | | | | | |
| likelihood) (Å) | | | | | | | |
| Ramachandran | | | | | | | |
| Plot | | | | | | | |
| Most favored | 98.1 | 97.5 | 97.6 | 98.7 | 97.8 | 97.6 | 98.1 |
| (%) | | | | | | | |
| Additionally | 1.9 | 2.5 | 2.4 | 1.3 | 2.2 | 2.2 | 1.9 |
| allowed (%) | | | | | | | |

¹Values in parenthesis are for the highest resolution shell.
²R_{merge} = ΣₕₖₗΣᵢ |Iᵢ(hkl) − <I(hkl)>|/ΣₕₖₗΣᵢ Iᵢ(hkl), where Iᵢ(hkl) is the intensity measured for the ith reflection and <I(hkl)> is the average intensity of all reflections with indices hkl.
³R_{factor} = Σₕₖₗ ||F_{obs} (hkl) | − |F_{calc} (hkl) ||/Σₕₖₗ |F_{obs} (hkl)|; R_{free} is calculated in an identical manner using 5% of randomly selected reflections that were not included in the refinement.
⁴Rₘₑₐₛ = redundancy-independent (multiplicity-weighted) R_{merge}. Rₚᵢₘ = precision-indicating (multiplicity-weighted) R_{merge}.
⁵CC_{1/2} is the correlation coefficient of the mean intensities between two random half-sets of data.

### Example 2

Several compounds were synthetized based upon the Series F compounds using the backbone I and screened against SARS-CoV-2 and MVERS-CoV as described in Example 1 above.

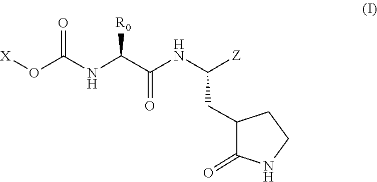

### Synthesis of F1 Series

The synthesis of the compounds shown follows previously published procedures (Heinrich et al J Med Chem 62 (2019) 11119-11134) to make the appropriate precursor carboxylic acids followed by treatment with carbonyl diimidazole and sodium borohydride to furnish the corresponding alcohols which were then used to make the inhibitors. Briefly, a solution of cyclopropyl Meldrum's acid (1.3 eq) in acetonitrile:DMF (10:3 mixture) was reacted with an appropriate amine (R—NH2, 1 eq) at 60° C. for 12 h. The solvents were removed in vacuo and the residue was dissolved in 10% aqueous sodium hydroxide and extracted with ethyl acetate. The layers were separated, and the aqueous layer was then acidified and extracted with ethyl acetate to yield the acid, which was dried thoroughly under vacuum. A solution of the acid (1 eq) in THE was treated with carbonyl diimidazole (1.5 eq) and stirred for 30 minutes. An aqueous solution of sodium borohydride (2.5 eq) was added dropwise and the mixture was stirred overnight. The solution was acidified, the solvent was removed in vacuo and the residue was extracted with ethyl acetate to yield the desired alcohol (91% yield). The reaction sequence shown in Scheme 1 was then used to generate the inhibitors.

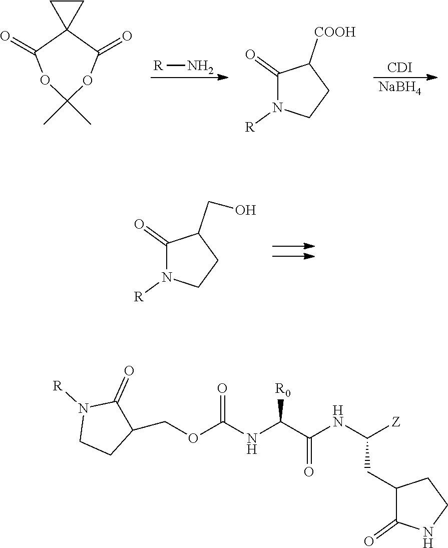

R₀ can be any natural or unnatural amino acid side chain (preferably leucine/isobutyl), and R can be any aliphatic or aromatic amine (substituted or unsubstituted), or a heterocyclic amine.

TABLE 6
Alcohol input

| | |
|---|---|
| | (F1) |
| 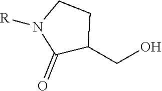 | |

| Com- | | | IC₅₀ (μM) | |
|---|---|---|---|---|
| pound | | | SARS- | MERS- |
| Code | [X] | Z | CoV-2 | CoV |
| 9b 9c | 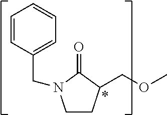 | CHO CH(OH)SO₃Na | 1.2 0.9 | 0.2 0.18 |

### Synthesis of F2 Series

A mixture of dimethyl itaconate (15 mmol), amine (15 mmol) and methanol (1.5 mL) was kept at RT overnight. The reaction mixture was then refluxed for 2 h and the solvent removed. Water (30 mL) was added to the residue and the mixture was extracted with ethyl acetate (3×30 mL). The combined organic extracts were dried over anhydrous sodium sulfate and the drying agent was filtered off. The filtrate was concentrated and the crude product was purified using flash chromatography (silica gel/ethyl acetate/hexane). Lithium borohydride reduction yielded the corresponding alcohols which were then used to make the inhibitors using Scheme 1.

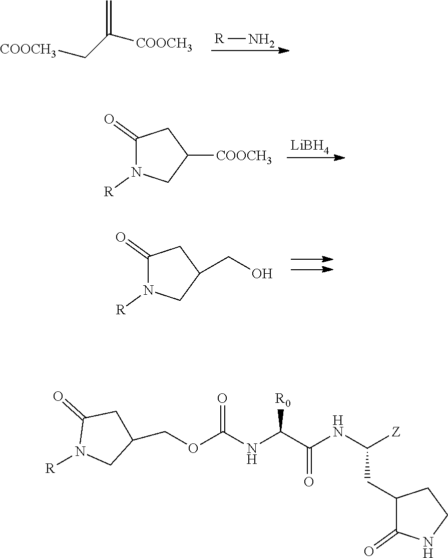

R₀ can be any natural or unnatural amino acid side chain (preferably leucine/isobutyl), and R can be any aliphatic or aromatic amine (substituted or unsubstituted), or a heterocyclic amine.

TABLE 7
Alcohol input

| | | | (F2) |
|---|---|---|---|
| 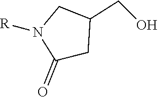 | | | |
| | | | IC₅₀ |
| | | | (μM) |
| Compound | | | SARS- |
| Code | [X] | Z | CoV-2 |
| 9b 9c | 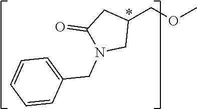 | CHO CH(OH)SO₃Na | 0.53 0.62 |
| 10b 10c | 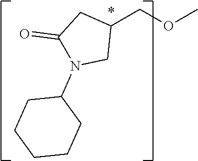 | CHO CH(OH)SO₃Na | TBD TBD |

### Synthesis of F3 Series

Briefly, (L)Boc-glutamic acid dimethyl ester (1 eq) was treated with 4M HCl in dioxane (10 eq) and stirred for 3 h. Removal of the solvent yielded the product, which was dissolved in methanol and treated with triethylamine (4 eq). After stirring for 30 minutes, the mixture was cooled to 0° C., followed by the addition of benzaldehyde (1.1 eq) and sodium borohydride (2 eq) and stirring continued at 0° C. for 2 h. A mixture of 20% HCl and ether (1:1 mixture) was added and the organic layer was removed in vacuo. The aqueous layer was neutralized with solid sodium carbonate and extracted with ether. The isolated amine was dissolved in acetonitrile and then refluxed for 4 h to yield the desired cyclized product. The ester was then treated with lithium borohydride (3 eq) to yield the alcohol which was used to generate the inhibitors as shown in Scheme 1.

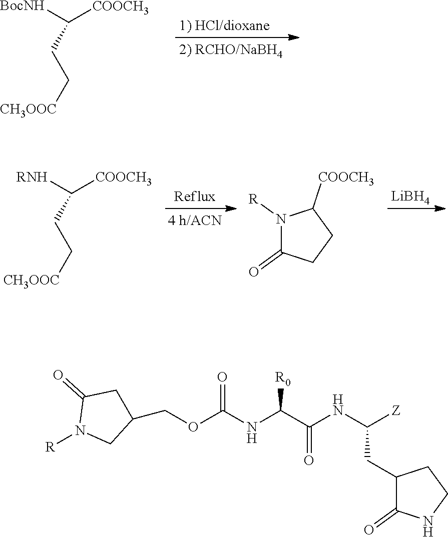

TABLE 8
Alcohol input

| | | | (F-3) |
|---|---|---|---|
| 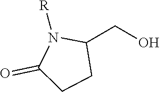 | | | |
| | | | IC₅₀ |
| Com- | | | (μM) |
| pound | | | SARS- |
| Code | [X] | Z | CoV-2 |
| 1b 1c 2c | 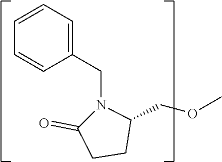 | CHO CH(OH)SO₃Na | 0.41 0.43 TBD |
| 3b 3c | 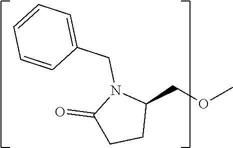 | CH(OH)SO₃Na | TBD TBD |

### Series F4 (1,3,2-Oxazaphospholidin-3-One) Synthesis

The general procedures for generating the P ring (first 3 steps) are reported in Thomson C M et al J Org Chem 55 (1990) 111-116. Briefly, (S) serine methyl ester hydrochloride (0.065 mmol) was dissolved in anhydrous methanol and cooled to 0° C. Triethylamine (0.065 mmol) was added and the reaction mixture was stirred for 10 minutes. Benzaldehyde (0.065 mmol) was added and the reaction mixture was stirred for 2 h, at which time sodium borohydride (0.13 mmol) was added portionwise over 30 minutes. The solution was partitioned between 20% HCl (50 mL) and ether (50 mL). The organic phase was extracted twice with 20-mL portions of 20% HCl and the combined aqueous layers were washed with ether (20 mL). The combined aqueous later was carefully neutralized with solid sodium carbonate and extracted with diethyl ether (3×20 mL). The combined ether extracts were washed with brine (30 mL) and dried over anhydrous sodium sulfate. Evaporation of the solvent yielded (S)-N-benzyl serine methyl ester (70% yield) which was used in the next step without further purification. A solution of (S)-benzyl serine methyl ester (4.78 mmol) in dry toluene (20 mL) was cooled to 0° C. and then treated with triethylamine (10 mmol) followed by phosphorous oxychloride (4.78 mmol) and stirred for 3 h. The reaction mixture was diluted with dry THE (50 mL) and the solution was filtered through a pad of Celite. Evaporation of the solvent yielded a syn- and anti-mixture of diastereomers (if desired, these can be separated by flash chromatography). Alternatively, a solution of the mixture in toluene (15 mL/g of starting material) was treated with an alcohol (R40H) (150 mol %) followed by the addition of triethylamine (100 mol %) with stirring. The reaction was monitored by TLC. The reaction mixture was partitioned between ethyl ether and saturated sodium carbonate. The organic phase was washed sequentially with aqueous sodium carbonate and brine. The ether layer was dried over anhydrous sodium sulfate, and the crude mixture of syn- and anti-methyl esters was purified by flash chromatography. Reduction of the esters with lithium borohydride yielded a mixture of the corresponding alcohols which was then used to synthesize the inhibitors (Scheme 1).

These compounds can exist in two distinct conformations (syn and anti), where the R₄ group is oriented in space in two different ways and therefore each series can access different regions of space in S₄ subsite. The two series can be isolated separately, although in the examples thus far, we generated them as a mixture of the syn and anti series (hence these were screened as mixtures).

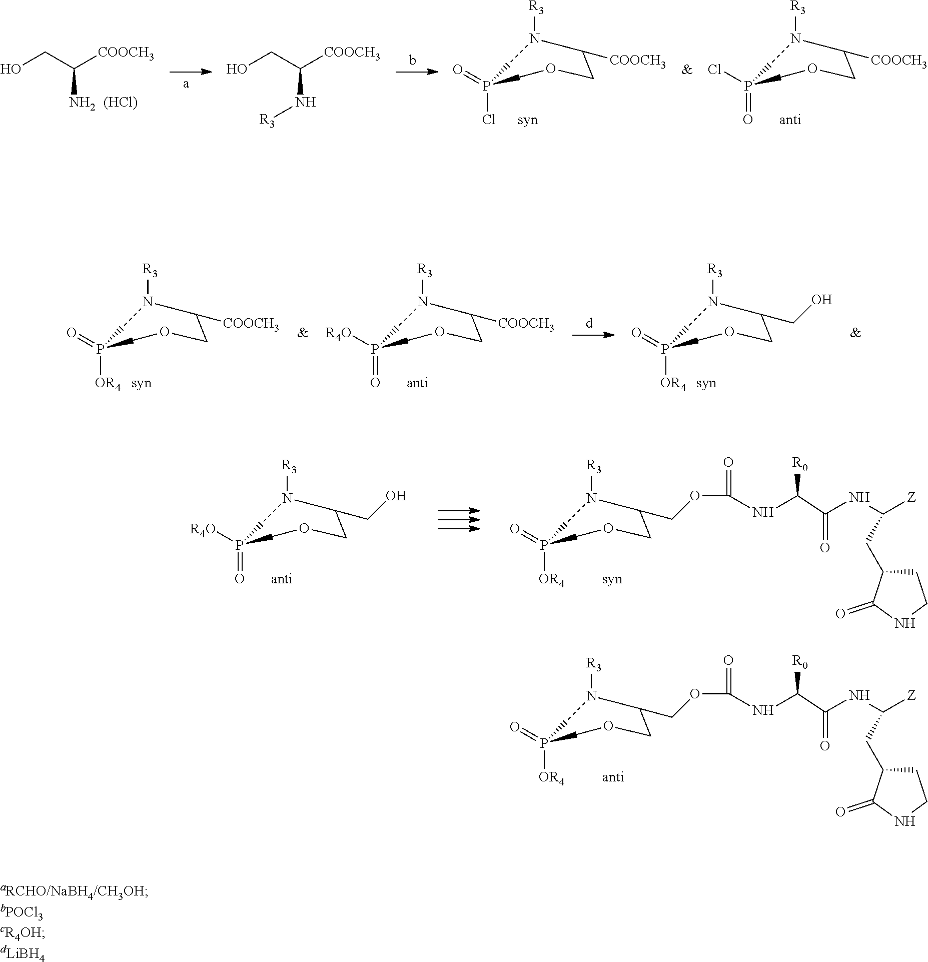

If the starting material used is threonine methyl ester hydrochloride, then the structure will have a methyl group which will be the R₅ group (R₅=methyl)

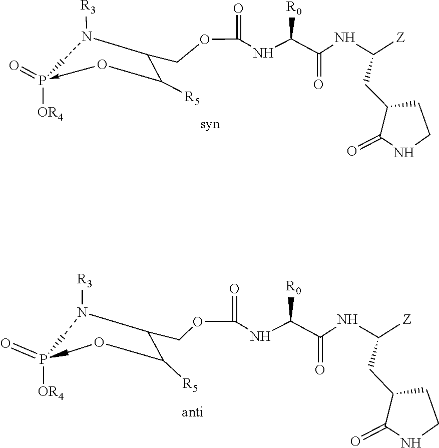

TABLE 9
Alcohol Input

| | | | | (F4) |
|---|---|---|---|---|
| 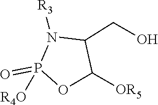 | | | | |
| Compound | | | IC50 (μM) | CC50 |
| Code | Structure | Z | SARS-CoV-2 | (μM) |
| 1b | R₃ = benzyl | CHO | 0.81 | >100 |
| Ic | R₄ = Isopropyl | CH(OH)SO₃Na | 0.845 | |
| | R₅ = H | | | >100 |
| 2b | R₃ = Isobutyl | CHO | 0.6 | |
| | R₄ = 4-Fluorobenzyl | | | |
| | R₅ = H | | | |

### Series F5 Synthesis

TABLE 10
Alcohol Inputs

| | |
|---|---|
| | (F5) |
| 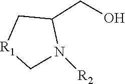 | |

| | | | IC₅₀ (μM) |
|---|---|---|---|
| Compound | | | SARS- |
| Code | [X] | Z | CoV-2 |
| 1b 1c | 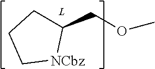 | CHO CH(OH)SO₃Na | 1.1 0.9 |
| 2b 2c | 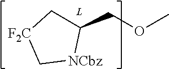 | CHO CH(OH)SO₃Na | 0.36 0.38 |
| 3b 3c | 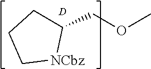 | CH(OH)SO₃Na | 0.27 0.24 |

### Example 3

### Series D (Azetidines) and E (Spirocycles)

We report herein the results of preliminary studies related to the structure-guided design of potent and permeable inhibitors of SARS-CoV-2 3CLpro that incorporate in their structure a spirocyclic component as a design element to optimally exploit new chemical space in the active site of the protease. Finally, for comparative purposes, a series of azetidine-derived inhibitors were also synthesized and evaluated in biochemical and cell-based assays.

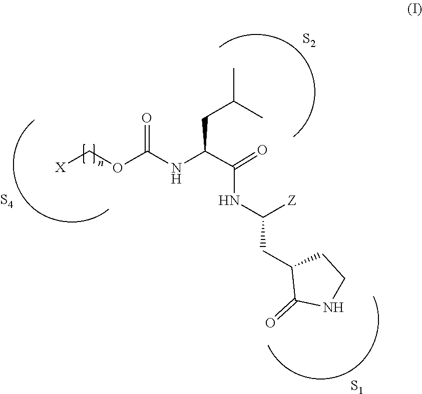

### Results and Discussion

Inhibitor design rationale. There are an array of advantages accrued from the judicious use of spirocycles in drug design, including improved physicochemical and PK characteristics, structural novelty, reduced conformational flexibility, and the capture of favorable binding interactions by probing and exploiting poorly-explored regions of chemical space. Importantly, the structural motifs embodied in spirocycles make possible the rigorous control of the spatial disposition of exit vectors; consequently, it was envisaged that the attachment of a suitably-decorated spirocycle capable of engaging in favorable binding interactions with the S₄ subsite region of SARS-CoV-2 3CLpro to a recognition element that is congruent with the known substrate specificity of the enzyme (in the case of SARS-CoV-2 3CLpro, a Leu-Gln surrogate fragment), would yield a molecule with high inhibitory prowess. The validity of the approach and the design of the inhibitors was further facilitated by the use of high resolution cocrystal structures. Lastly, we sought to harness the benefits accrued through deuteration, particularly the potential improvement of pharmacokinetics and physicochemical properties, consequently, a select number of deuterated inhibitors are also made. For the general spirocyclic and azetidine alcohol inputs for these Series see Table 1.

Chemistry. The inhibitors were synthesized by attaching an azetidine-based spirocyclic alcohol to a Leu-Gln surrogate fragment incorporating an aldehyde warhead or latent aldehyde bisulfite adduct. The spirocyclic and azetidine-based precursor alcohols were either commercially available or readily synthesized using commercially available ketone or carboxylic acid precursors.

The appropriate spirocyclic and azetidine alcohol inputs (Tables 11 and 12) were treated with N, N′-disuccinimidyl carbonate (DSC), followed by coupling of the resulting mixed carbonate to amino alcohol A. Dess-Martin periodinane oxidation of dipeptidyl alcohol a generated the desired aldehydes b which were subsequently transformed into the corresponding aldehyde bisulfite adducts c (see Scheme 1).

Biochemical studies. The inhibitory activity of the compounds toward SARS-CoV-2 3CL protease in biochemical and cell-based assays, as well as the cytotoxicity of the compounds, are determined and the results are listed in Tables 11 and 12. For comparative purposes, the interaction of a select number of compounds with MERS-CoV-2 3CL protease is also investigated. Selected compounds were tested in a cell-based assay against SARS-CoV-2 as described in the experimental section. The IC₅₀ values, EC₅₀ values for a select number of inhibitors, and the CC₅₀ values in CRFK cells are summarized in Tables 11 and 12 and they are the average of at least two determinations

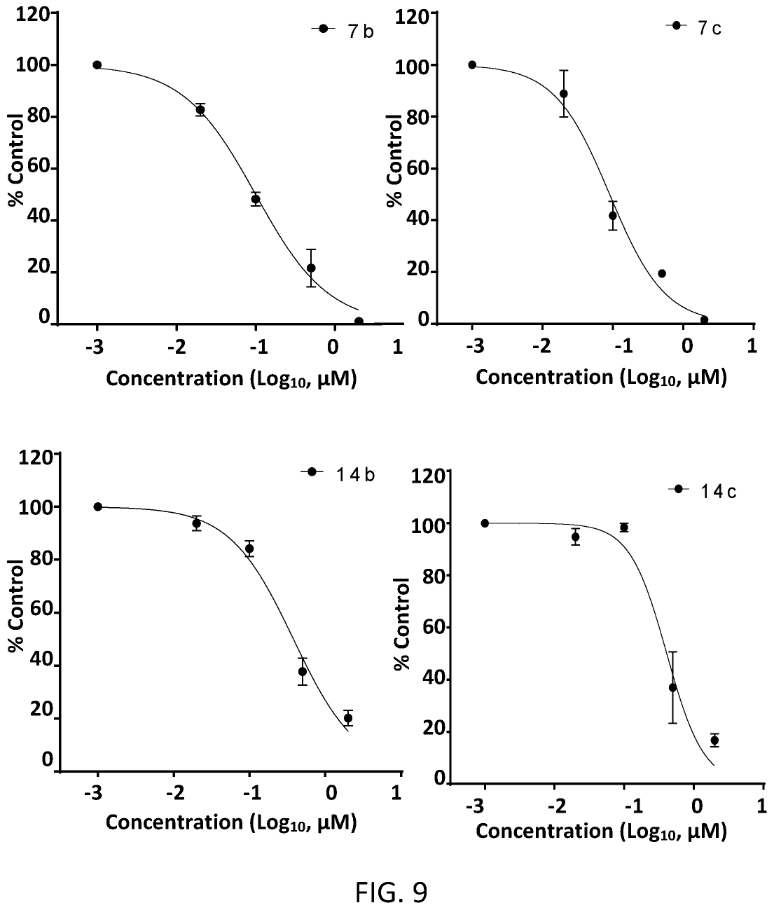

In this study, we used another BSL2 cell-based replicon assay in 293T cells, mimicking the natural cycle of SARS-CoV-2 replication. As a control, we used GC376 and the EC₅₀ was calculated at 0.037±0.01 μM in the assay. The EC₅₀ is comparable to the value (0.02 μM in 293T cells) previously reported with the same system. Four compounds were selected for the determination of EC₅₀s, and inhibition curves by each compound were consistent with a dose-dependent mode and R²>0.9 (FIG. 9). The selected compounds were potent SARS-CoV-2 inhibitors with EC₅₀ values ranging from 0.08 to 0.43 μM (Tables 11 and 12). These were correlated well with IC₅₀ values.

TABLE 11
IC₅₀ values of spirocyclic inhibitors 1-11b/c against SARS-CoV-2 3CL^{pro} and MERS-CoV
3CL^{pro}, and CC₅₀ values.

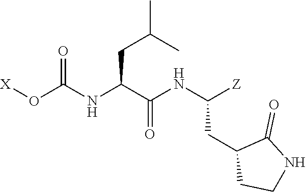

| | | | IC₅₀ (μM) | | |
|---|---|---|---|---|---|
| Compound | | | SARS-CoV-2 | MERS-CoV | CC₅₀ |
| Code | X— | Z | 3-CL^{pro} | 3CL^{pro} | (μM) |
| 1b 1c | 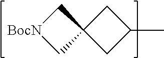 | —CHO —CH(OH)SO₃ | 3.30 ± 0.28 0.85 ± 0.07 | 1.50 ± 0.42 0.28 ± 0.11 | >100 >100 |
| 2b 2c | 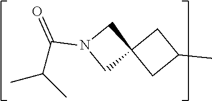 | —CHO —CH(OH)SO₃ | 1.15 ± 0.49 0.65 ± 0.07 | 0.35 ± 0.07 0.55 ± 0.07 | >100 >100 |
| 3b 3c | 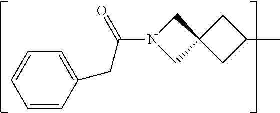 | —CHO —CH(OH)SO₃ | 0.26 ± 0.04 0.26 ± 0.05 | 0.21 ± 0.04 0.13 ± 0.03 | >100 >100 |
| 4b 4c | 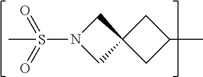 | —CHO —CH(OH)SO₃ | 0.68 ± 0.04 0.76 ± 0.08 | 0.22 ± 0.03 0.16 ± 0.01 | >100 >100 |
| 5b 5c | 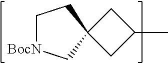 | —CHO —CH(OH)SO₃ | 0.45 ± 0.05 0.75 ± 0.07 | 0.70 ± 0.14 0.75 ± 0.07 | >100 >100 |
| 6b 6c | 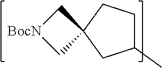 | —CHO —CH(OH)SO₃ | 1.25 ± 0.07 1.20 ± 0.14 | 1.01 ± 0.27 1.30 ± 0.14 | >100 >100 |
| 7b^{#} 7c^{#} | 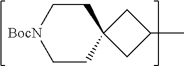 | —CHO —CH(OH)SO₃ | 0.36 ± 0.06 0.29 ± 0.02 | 1.20 ± 0.14 0.95 ± 0.21 | >100 >100 |
| 8b 8c | 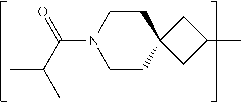 | —CHO —CH(OH)SO₃Na | 0.38 ± 0.04 0.41 ± 0.01 | 0.65 ± 0.21 0.52 ± 0.12 | >100 >100 |
| 9b 9c | 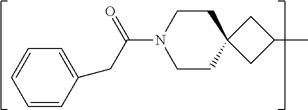 | —CHO —CH(OH)SO₃Na | 0.35 ± 0.07 0.29 ± 0.06 | 0.70 ± 0.14 0.61 ± 0.13 | >100 >100 |
| 10b 10c | 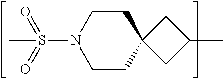 | —CHO —CH(OH)SO₃Na | 0.24 ± 0.01 0.24 ± 0.03 | 0.37 ± 0.05 0.33 ± 0.04 | >100 >100 |
| 11b 11c | 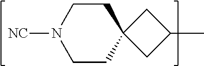 | —CHO —CH(OH)SO₃Na | 0.32 ± 0.05 0.39 ± 0.03 | 0.56 ± 0.06 0.63 ± 0.18 | >100 >100 |

^{#}The EC₅₀ values of the aldehyde and bisulfite salt adduct were determined to be 0.09 ± 0.01 and 0.08 ± 0.02, respectively.

TABLE 12
IC₅₀ values of azetidine inhibitors 12-17b/c against SARS-CoV-2 3CL^{pro} and MERS-CoV 3CL^{pro}, and CC₅₀ values.

| | | | IC₅₀ (μM) | | |
|---|---|---|---|---|---|
| Compound | | | SARS-CoV-2 | MERS- | |
| Code | Structure | Z | 3CL^{pro} | CoV 3CL^{pro} | CC₅₀ (μM) |
| 12b 12c | 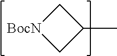 | —CHO —CH(OH)SO₃N | 2.50 ± 0.28 3.05 ± 0.35 | 1.65 ± 0.64 2.55 ± 0.92 | >100 >100 |
| 13b 13c | 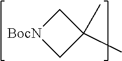 | —CHO —CH(OH)SO₃N | 3.65 ± 0.64 2.50 ± 0.57 | 3.45 ± 1.20 4.30 ± 0.28 | >100 >100 |
| 14b* 14c* | 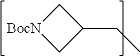 | —CHO —CH(OH)SO₃N | 0.41 ± 0.04 0.05 ± 0.14 | 0.49 ± 0.04 0.44 ± 0.06 | >100 >100 |
| 15b 15c | 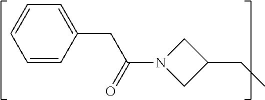 | —CHO —CH(OH)SO₃Na | 0.83 ± 0.04 0.76 ± 0.08 | 0.28 ± 0.11 0.18 ± 0.01 | >100 >100 |
| 16b 16c | 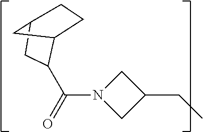 | —CHO —CH(OH)SO₃Na | 0.52 ± 0.14 0.49 ± 0.02 | 0.19 ± 0.04 0.17 ± 0.03 | >100 >100 |
| 17b 17c | 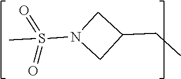 | —CHO —CH(OH)SO₃Na | 4.95 ± 0.49 4.05 ± 0.78 | 1.40 ± 0.14 1.35 ± 0.21 | >100 >100 |

^{#}The EC₅₀ values of the aldehyde and bisulfite salt adduct were determined to be 0.38 ± 0.07 and 0.43 ± 0.16, respectively.

### X-Ray Crystallographic Studies

In order to gain insight and understanding into the binding of the spirocyclic inhibitors to the active site of the protease, as well as identify the structural determinants associated with binding, high-resolution cocrystal structures of SARS-CoV-2 3CL^{pro} and MERS-CoV 3CL^{pro} were obtained in complex with spirocyclic and azetidine-derived inhibitors. For all structures described below, the electron density was consistent both the R and S enantiomers at the stereocenter formed by covalent attachment of the Sγ atom of Cys 145 or Cys 148 in SARS-CoV-2 3CL^{pro} and MVERS-CoV 3CL^{pro}, respectively. Therefore, the alternate conformations were modeled as each enantiomer with 0.5 occupancy.

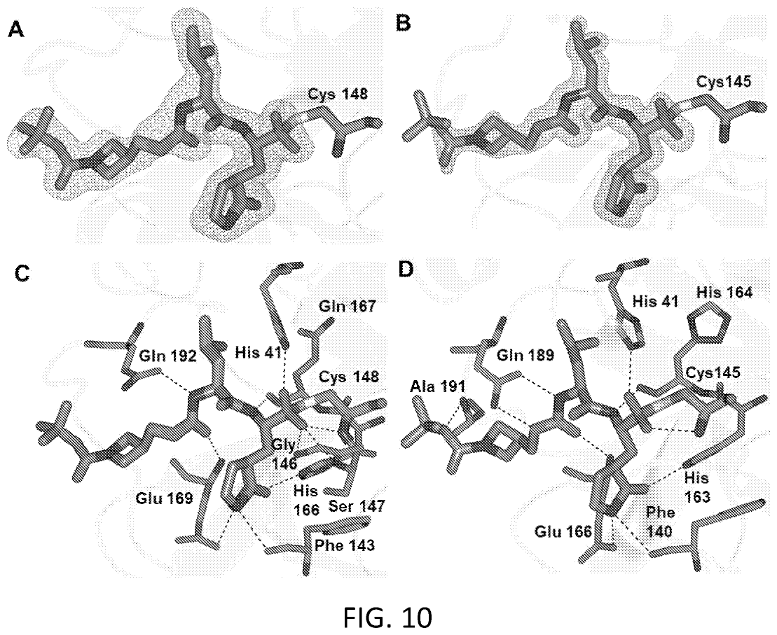

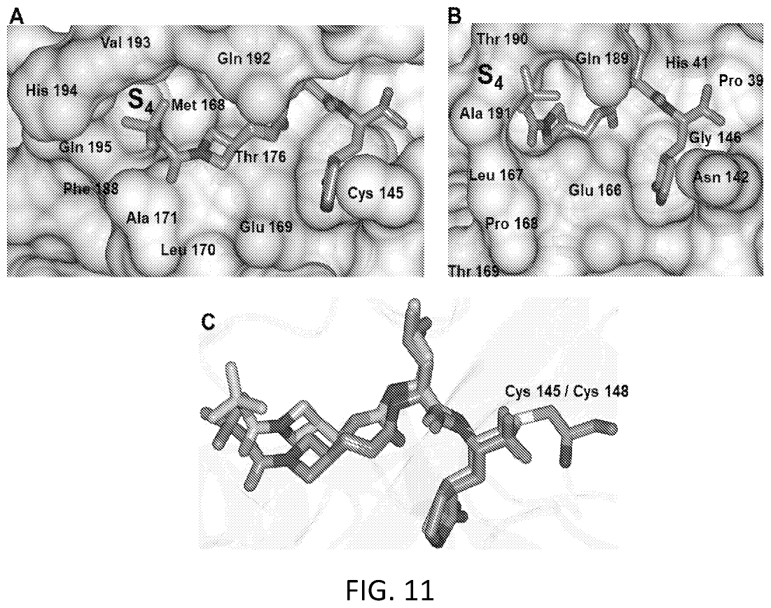

Azetidine-derived inhibitor bound structures. In the case of the azetidine inhibitor 14c, the active site contained prominent difference electron density consistent with the inhibitor covalently bound to Cys 148 and Cys 145 in each subunit (FIGS. 10A and 10B). Inhibitor 14c forms the typical hydrogen bonds to MERS-CoV 3CLP^{pro} and SARS-CoV-2 3CLpro (FIGS. 4C and 4D) along with an additional contact to the backbone nitrogen atom of Ala 191 in the case of SARS-CoV-2 3CLpro. This places the inhibitor deep within the S₄ subsites as shown in FIGS. 11A and 11B. Superposition of the two structures revealed similar binding modes although the azetidine rings are rotated in the S₄ subsite approximately 900 relative to one another (FIG. 11C).

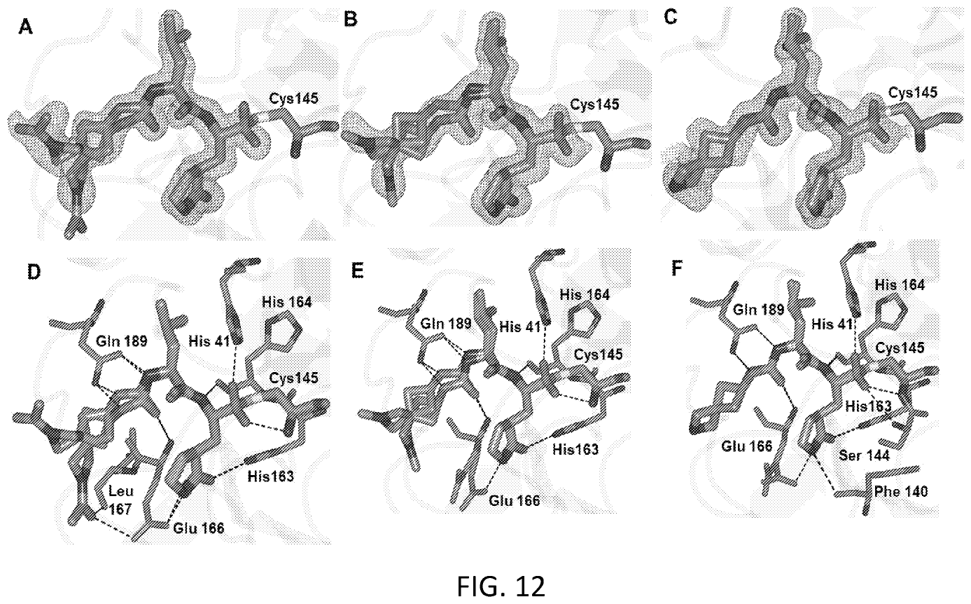

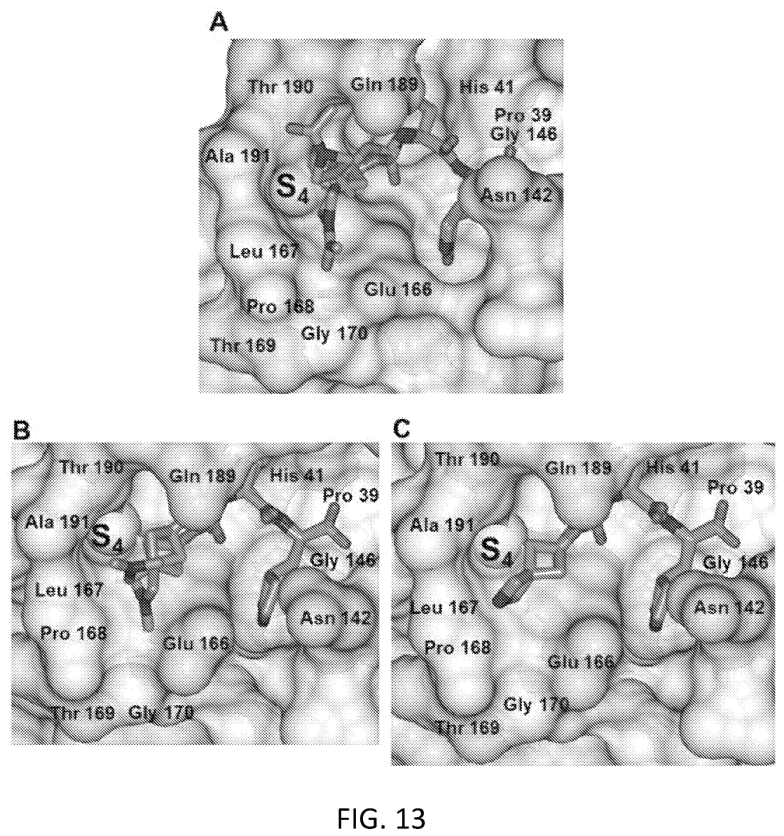

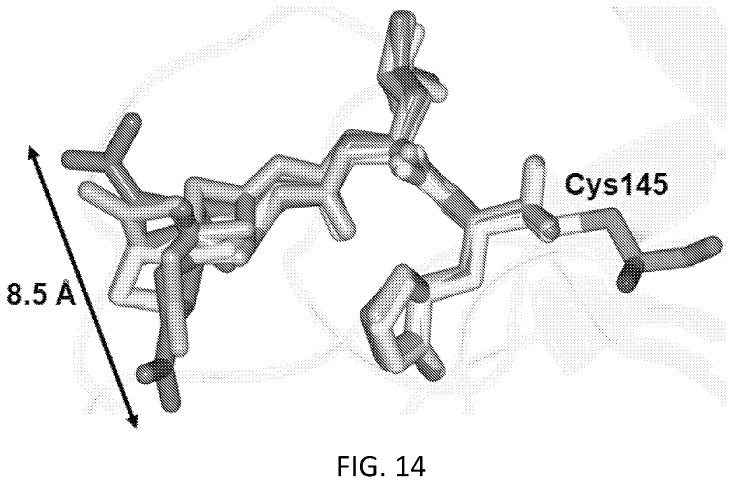

2-Azaspiro [3.3]-derived inhibitor bound structures. Similar to the azetidine inhibitors above, difference electron density consistent with inhibitors 2c, 3c and 4c bound in the SARS-CoV-2 3CLpro active site covalently to Cys 145 (FIG. 12A-C). For 2c, the spirocyclic portion of the inhibitor that binds in the S4 subsite appears to adopt two conformations based on the electron density (FIG. 12A). However, the isopropyl groups were disordered in both conformations. Inhibitor 3c, also adopted two conformations (FIG. 12B) but the benzyl ring at the terminal end was disordered and could not be modeled. Interestingly, 4c appeared to adopt one conformation in the spirocyclic region of the inhibitor although electron density for the methyl sulfonyl group was not present, which indicated a certain degree of disorder in this region. The inhibitors form the typical hydrogen bonds to the protein (FIG. 12B) with an additional polar contact observed between the carbonyls of 2c and Leu 167. The wide conformational differences in these inhibitors positions the spirocyclic regions over a wide region of space within the S4 subsite as shown in FIG. 13. Overall, superposition of these structures revealed a high degree of similarity in the ligand conformations. However, as evident in FIG. 14, a large degree of motion is present in the spirocyclic region of the compounds with the largest span covering 8.5 Å in the case of inhibitor 2c.

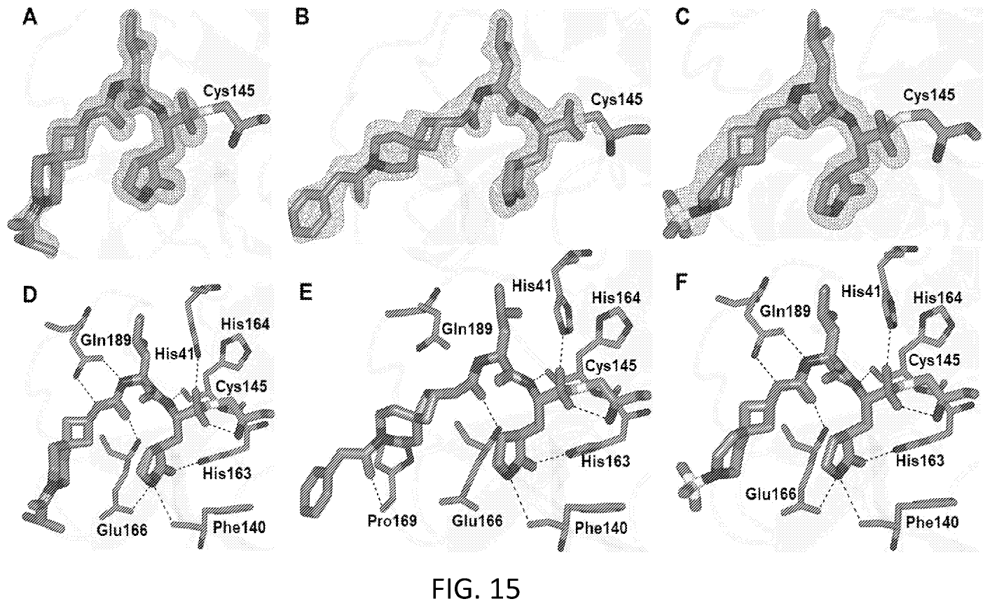

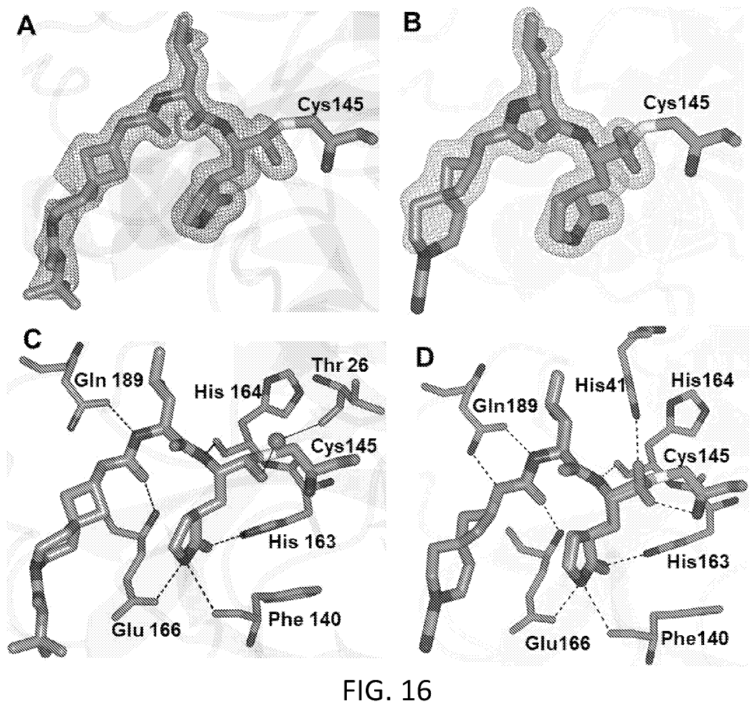

6-Azaspiro [3.5]-derived inhibitor bound structures. Interestingly, the spirocyclic inhibitors that contained the larger 6-membered nitrogen heterocycle did not display the same degree of disorder observed for 2c, 3c and 4c, which contain the 4-membered rings. This was revealed by the structure determination of 7c, 8c, 9c, 10c and 11c in complex with SARS-CoV-2 3CL^{Pro} in which the electron density was well-defined for the majority of these inhibitors (FIG. 15A-C and FIG. 16 A-B). These inhibitors form similar hydrogen bond interactions with the protein that are typically observed which include His 41, His 163, His 164, Glu 166, Gln 189 and bifurcated H-bonds between Glu 166 and Phe 140 and the NH of the S-lactam ring (FIG. 15 D-F and FIG. 16 C-D). However, the structure with 9c adopts an additional polar contact (2.81 Å) between the carbonyl and the backbone carbonyl of Pro 168 (FIG. 15E).

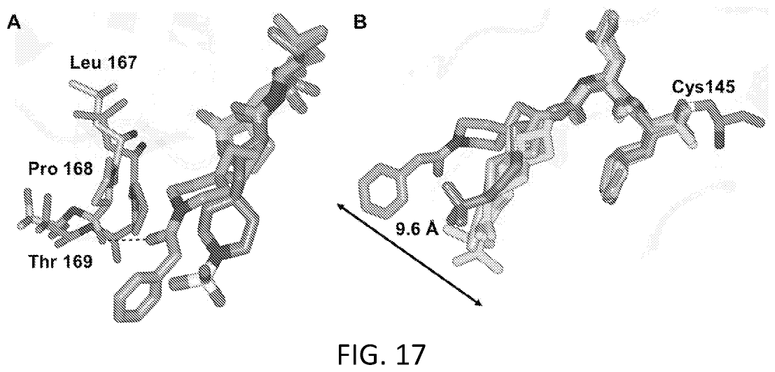

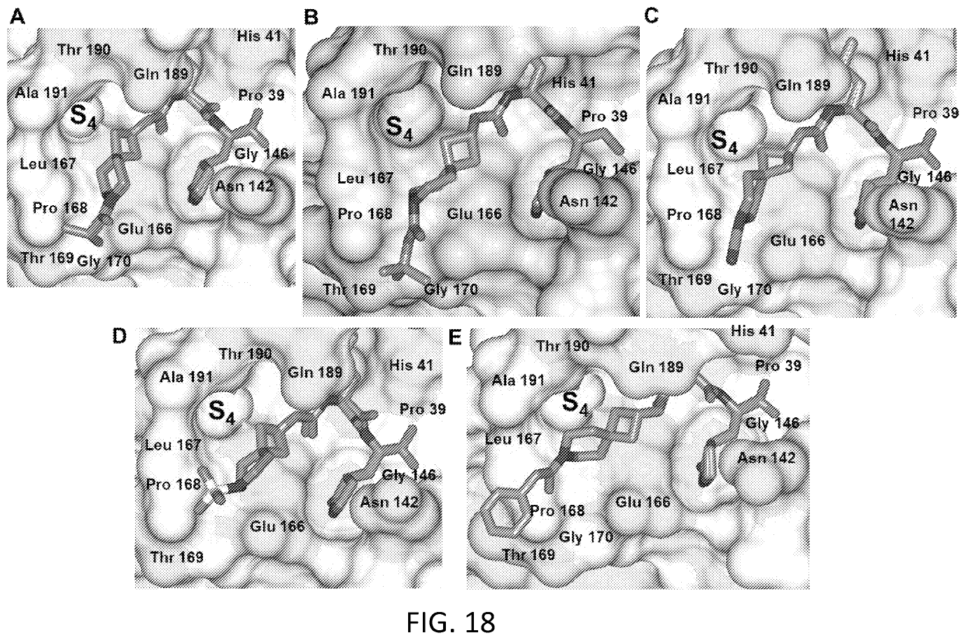

Notably, the methyl sulfonyl group of 10c is in proximity to Pro 168 but too far to form an interaction (3.4 Å). The interaction between Pro 168 and 9c results in the movement (˜2.6 Å) of a nearby loop that includes Leu 167, Pro 168 and Thr 169 relative to the other structures, such as 10c (FIG. 17A). Overall, the structures with 7c, 8c and 11c adopt very similar binding modes (FIG. 17B) in which the terminal ends of the inhibitors are positioned between a cleft formed by Glu 166 and Pro 168 (FIG. 18 A-C). Inhibitor 10c is in an intermediate position as it is closer to Pro 168 within the hydrophobic ridge of the S4 subsite and 9c is the extreme case in which the benzyl ring is located on top of this ridge (FIG. 18 D-E). As a whole, these inhibitors occupy a wide range of space within the S4 subsite spanning approximately 9.5 Å (FIG. 17B).

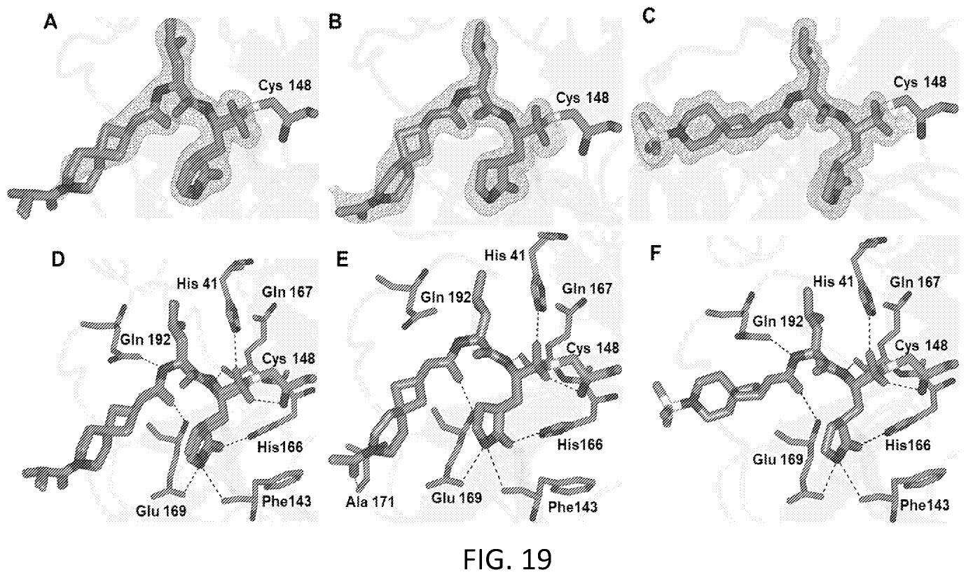

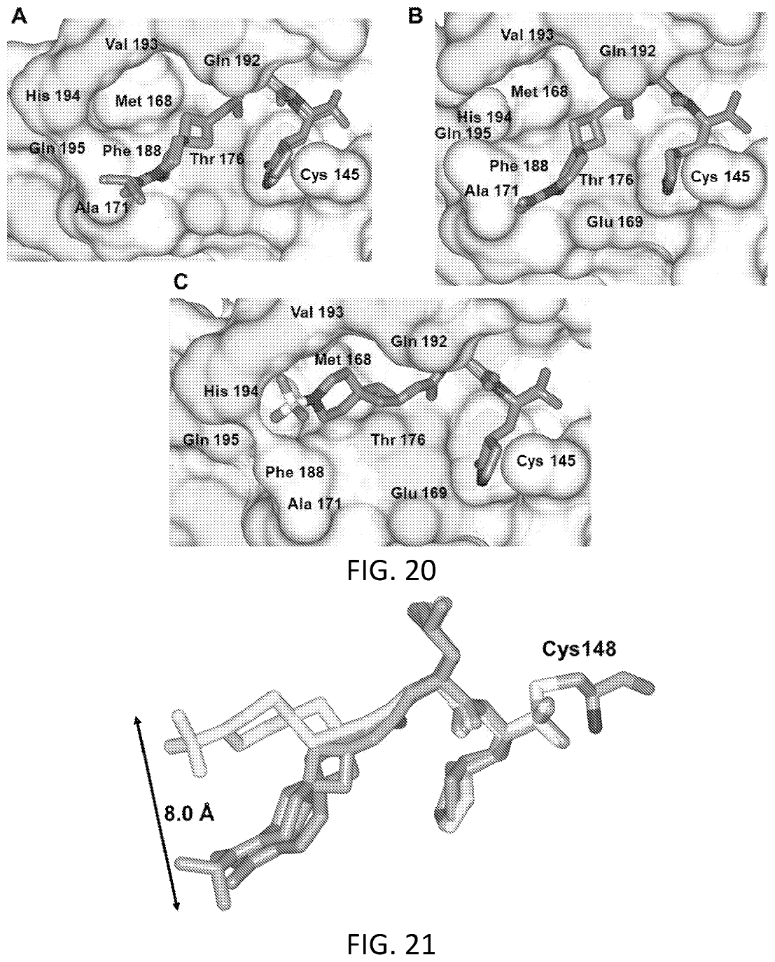

Similarly, the structures of MERS-CoV 3CL^{pro} with 8c, 9c and 10c yielded well-defined electron density overall (FIG. 19 A-C) although the benzyl ring was disordered in 9c. The inhibitors form the typical array of hydrogen bond interactions with the protein, including Glu 169, His 41, His 166 and bifurcated H-bonds between Glu 169 and Phe 143 and the NH of the S-lactam ring of the inhibitor (FIG. 19 D-F). For the structure with 9c, an additional polar contact with the backbone carbonyl of Ala 171 (3.07 Å) positions the molecule in the S₄ subsite in a similar pose as observed for 8c (FIG. 20 A-B). Although the carbonyl in the structure of 8c is in a similar orientation as 9c, the distance to the backbone carbonyl of Ala 171 is much larger (4.07 Å). The binding mode of 10c differs from 8c and 9c in that the methyl sulfonyl group is positioned deeper within the S4 subsite (FIG. 20 C) and is positioned 3.4 Å from His 194 potentially forming a salt bridge like interaction. The superimposed structures of MERS-CoV 3CLP^{pro} in complex with 8c, 9c and 10c are shown in FIG. 21 show that these inhibitors span a space within the S₄ subsite of approximately 8.0 Å. Collectively, the structural studies suggest that the use of spirocycles with different exit vectors are well-suited to exploiting new chemical space in and around the S₄ subsite.

Structure-Activity Relationships. A representative series of spirocyclic inhibitors derived from 2-azaspiro[3.3]-, 2-azaspiro[3.4]-, 6-azaspiro[3.4]-, and 6-azaspiro[3.5]-spirocycles displaying different exit vectors were synthesized and evaluated in biochemical and cell-based assays. It is evident from the results shown in Table 11 that the synthesized compounds generally display high inhibitory activity toward SARS-CoV-2 3CL^{pro} and MERS-CoV 3CL^{pro}, with the IC₅₀ values of most of the inhibitors in the submicromolar range. Furthermore, the compounds are devoid of cytotoxic effects. The IC₅₀ values of spirocycles 7b and 3b were found to be >9-fold and nearly 13-fold lower than that of compound 1b, respectively, suggesting that directional and recognition effects associated with the nature of the spirocycle and X group, respectively, are important in enhancing potency. The importance of exit vectors is also evident in comparing the relative potency of aldehyde inhibitors 1b, 5b and 6b which are derived from different spirocycles. The potency of compounds 8b, 9b, 10b and 11b was high and remained invariant to the nature of the R group. Several of the inhibitors were found to be broadly active against both SARS-CoV-2 3CLpro and MERS-CoV 3CL^{pro}, suggesting a high likelihood for identifying a broad-spectrum pre-clinical candidate. The EC₅₀ values of the aldehyde and corresponding bisulfite adduct pairs tested were comparable, and one pair was in the nM range (Table 11, compounds 7b/7c) The Safety Index (SI), defined as CC₅₀/EC₅₀, for the compounds was very high (˜1250). The results shown in Table 11 are congruent with the crystallographic studies (vide supra) and validate the use of spirocyclic inhibitors in exploring and exploiting new chemical space in the S₄ region of SARS-CoV-2 3CL^{pro}.

In the azetidine series, biochemical evaluation of the synthesized azetidine inhibitors revealed that the compounds were fairly potent against both SARS-CoV 3CLP^{pro} and MERS-CoV 3CLpro (Table 12). The IC₅₀ values of compounds 14b/14c having an extra methylene group were >6-fold better than those of the 12b/12c pair. Furthermore, in the series of compounds 14b, 15b, 16b and 17b, potency was found to be sensitive to the nature of the group attached to the azetidine nitrogen, with compound 14b being 12-fold more potent than 17b and with an EC₅₀ value of 0.38 μM.

### CONCLUSIONS

There is currently a need for the development of direct-acting antivirals to complement the use of vaccines and biologics for the treatment of COVID-19. In this study we have sought to exploit the directional and stereochemical control afforded by spirocycles to optimize potency. The results indicate that the incorporation of spirocyclic elements embellished with appropriate recognition moieties, combined with structural information gained from cocrystal structures, into the design of process, has resulted in the identification of highly effective broad-spectrum inhibitors of SARS-CoV-2 3CLpro and MERS-CoV 3CL^{pro}, with EC₅₀ values and Safety Indices in the 0.08-0.43 μM and >2000 range, respectively. The structural determinants associated with binding and the mechanism of action involving participation of the catalytic dyad Cys145 and His41 and the formation of a tetrahedral adduct, were elucidated using X-ray crystallography. These studies provide a solid foundation for conducting further preclinical studies.

### EXPERIMENTAL SECTION

### General

Reagents and dry solvents were purchased from various chemical suppliers (Advanced ChemBlocks, Sigma-Aldrich, Acros Organics, Chem-Impex, TCI America, Oakwood chemical, APExBIO, SynQuest, Fisher and Bachem) and were used as obtained. The synthesized compounds were purified using flash chromatography and silica gel (230-450 mesh) (Sorbent Technologies, Atlanta, GA). Normal phase chromatography was performed on a Teledyne ISCO CombiFlash system using RediSep normal phase silica cartridges (35-70 μm particle size range). Thin layer chromatography was performed using Analtech silica gel plates. Visualization was accomplished using UV light and/or iodine. ¹H NMR spectra were recorded in CDCl₃ or DMSO-d₆ using a Varian XL-400 spectrometer. Chemical shifts and coupling constants are reported in parts per million and hertz, respectively. The following abbreviations are used to describe splitting patterns: s, singlet; d, doublet; t, triplet; q, quartet; m, multiplet; br, broad. The purity of the final compounds was found to be ≥90%, as determined by absolute qNMR analysis using a Bruker AV III 500 NMR spectrometer equipped with a CPDUL CRYOprobe and CASE autosampler (the University of Kansas Nuclear Magnetic Resonance Laboratory). Dimethyl sulfone TraceCERT® was used as the internal calibrant. High resolution mass spectrometry (HRMS) was performed at the Wichita State University Mass Spectrometry lab using Orbitrap Velos Pro mass spectrometer (ThermoFisher, Waltham, MA) equipped with an electrospray ion source.

### Synthesis of Compounds

Preparation of compounds 1-17a. General procedure. To a solution of alcohol (1 eq) (Table 11) in anhydrous acetonitrile (10 mL/g alcohol) was added N,N′-disuccinimidyl carbonate (1.2 eq) and TEA (3.0 eq) and the reaction mixture was stirred for 4 h at room temperature. The solvent was removed in vacuo and the residue was dissolved in ethyl acetate (40 mL/g alcohol). The organic phase was washed with saturated aqueous NaHCO₃(2×20 mL/g alcohol), followed by brine (20 mL/g alcohol). The organic layers were combined and dried over anhydrous Na₂SO₄, filtered and concentrated in vacuo to yield the mixed carbonate which was used in the next step without further purification.

To a solution of Leu-Gln surrogate amino alcohol A (1.0 eq) in dry methylene chloride (10 mL/g of amino alcohol) was added TEA (1.5 eq) and the reaction mixture was stirred for 20 min at room temperature (solution 1). In a separate flask, the mixed carbonate was dissolved in dry methylene chloride (10 mL/g of carbonate) (solution 2). Solution 1 was added to solution 2 and the reaction mixture was stirred 3 h at room temperature. Methylene chloride was added to the organic phase (40 mL/g of carbonate) and then washed with saturated aqueous NaHCO₃(2×20 mL/g alcohol), followed by brine (20 mL/g alcohol). The organic phase was dried over anhydrous Na₂SO₄, filtered and concentrated in vacuo. The resultant crude product was purified by flash chromatography (hexane/ethyl acetate) to yield dipeptidyl alcohol a as a white solid.

Preparation of compounds 1-17b. General procedure. To a solution of dipeptidyl alcohol a (1 eq) in anhydrous dichloromethane (300 mL/g dipeptidyl alcohol) kept at 0-5° C. under a N₂ atmosphere was added Dess-Martin periodinane reagent (3.0 eq) and the reaction mixture was stirred for 3 h at 15-20° C. The organic phase was washed with 10% aq Na₂S₂O₃ (2×100 mL/g dipeptidyl alcohol), followed by saturated aqueous NaHCO₃(2×100 mL/g dipeptidyl alcohol), distilled water (2×100 mL/g dipeptidyl alcohol), and brine (100 mL/g dipeptidyl alcohol). The organic phase was dried over anhydrous Na₂SO₄, filtered and concentrated in vacuo. The resulting crude product was purified by flash chromatography (hexane/ethyl acetate) to yield aldehyde b as a white solid.

Tert-butyl 6-((((S)-4-methyl-1-oxo-1-(((S)-1-oxo-3-((R)-2-oxopyrrolidin-3-yl)propan-2-yl)amino)pentan-2-yl)carbamoyl)oxy)-2-azaspiro[3.3]heptane-2-carboxylate (1b). ¹H NMR (500 MHz, DMSO-d6) δ 9.38 (d, J=6.9 Hz, 1H), 8.44 (d, J=7.6 Hz, 1H), 7.53 (s, 1H), 7.33 (d, J=8.1 Hz, 1H), 4.74-4.60 (m, 1H), 4.08-3.89 (m, 2H), 3.81 (d, J=26.2 Hz, 4H), 3.19-3.04 (m, 2H), 2.30-2.02 (m, 7H), 1.98-1.74 (m, 2H), 1.71-1.38 (m, 3H), 1.36 (s, 9H), 0.86 (ddd, J=14.0, 10.5, 6.4 Hz, 6H). Yield (74%). HRMS m/z: [M+Na]⁺ Calculated for C₂₅H₄₀N₄NaO₇ 531.2795; Found 531.2776.

2-Isobutyryl-2-azaspiro[3.3]heptan-6-yl ((S)-4-methyl-1-oxo-1-(((S)-1-oxo-3-((R)-2-oxopyrrolidin-3-yl)propan-2-yl)amino)pentan-2-yl)carbamate (2b). Yield (24%). ¹H NMR (400 MHz, cdcl₃) δ 9.58 (s, 1H), 6.69 (s, 1H), 5.88 (s, 1H), 5.68 (s, 1H), 5.23-4.79 (m, 2H), 4.38-4.09 (m, 2H), 4.02-3.89 (m, 2H), 3.78-3.66 (m, 2H), 3.63-3.54 (m, 2H), 3.51-3.24 (m, 4H), 2.69-2.19 (m, 2H), 2.19-1.98 (m, 1H), 1.98-1.37 (m, 5H), 1.19-1.10 (m, 6H), 1.03-0.79 (m, 6H). HRMS m/z: [M+Na]⁺ Calculated for C₂₄H₃₈N₄NaO₆ 501.2689; Found 501.2672.

2-(2-Phenylacetyl)-2-azaspiro[3.3]heptan-6-yl ((S)-4-methyl-1-oxo-1-(((S)-1-oxo-3-((R)-2-oxopyrrolidin-3-yl)propan-2-yl)amino)pentan-2-yl)carbamate (3b). Yield (69%). ¹H NMR (400 MHz, cdcl₃) δ 9.46 (s, 1H), 8.95 (d, J=5.1 Hz, 1H), 7.40-7.22 (m, 5H), 6.61 (s, 1H), 5.87 (s, 1H), 5.17 (d, J=8.5 Hz, 1H), 4.94-4.86 (m, 1H), 4.35-4.25 (m, 1H), 4.25-4.17 (m, 1H), 3.63-3.53 (m, 2H), 3.53-3.39 (m, 4H), 3.37-3.29 (m, 2H), 2.51-2.35 (m, 2H), 2.33-2.10 (m, 2H), 2.09-1.96 (m, 1H), 1.94-1.62 (m, 5H), 1.57-1.44 (m, 1H), 1.01-0.89 (m, 6H). HRMS m/z: [M+Na]⁺ Calculated for C₂₈H₃₈N₄NaO₆ 549.2689; Found 549.2675.

2-(Methylsulfonyl)-2-azaspiro[3.3]heptan-6-yl ((S)-4-methyl-1-oxo-1-(((S)-1-oxo-3-((R)-2-oxopyrrolidin-3-yl)propan-2-yl)amino)pentan-2-yl)carbamate (4b). Yield (16%). ¹H NMR (400 MHz, cdcl₃) δ 9.45 (s, 1H), 8.12 (s, 1H), 6.67 (s, 1H), 6.24 (s, 1H), 5.03-4.79 (m, 1H), 4.23 (t, J=11.4 Hz, 1H), 4.00-3.87 (m, 1H), 3.71-3.54 (m, 4H), 3.44-3.16 (m, 6H), 2.99 (s, 3H), 2.52-2.28 (m, 2H), 2.26-1.71 (m, 3H), 1.68-1.45 (m, 3H), 1.05-0.78 (m, 6H). HRMS m/z: [M+Na]⁺ Calculated for C₂₁H₃₄N₄NaO₇S 509.2046; Found 509.1988.

Tert-butyl 2-((((S)-4-methyl-1-oxo-1-(((S)-1-oxo-3-((R)-2-oxopyrrolidin-3-yl)propan-2-yl)amino)pentan-2-yl)carbamoyl)oxy)-6-azaspiro[3.4]octane-6-carboxylate (5b). Yield (88%). 1H NMR (400 MHz, cdcl₃) δ 9.49 (s, 1H), 8.34 (s, 1H), 6.06 (s, 1H), 5.28-5.17 (m, 1H), 5.02-4.89 (m, 1H), 4.38-4.12 (m, 2H), 3.47-3.19 (m, 6H), 2.55-2.29 (m, 4H), 2.19-1.80 (m, 7H), 1.80-1.61 (m, 2H), 1.61-1.49 (m, 1H), 1.45 (s, 9H), 1.01-0.89 (m, 6H). HRMS m/z: [M+Na]⁺ Calculated for C₂₆H₄₂N₄NaO₇ 545.2951; Found 545.2931.

Tert-butyl 6-((((S)-4-methyl-1-oxo-1-(((S)-1-oxo-3-((R)-2-oxopyrrolidin-3-yl)propan-2-yl)amino)pentan-2-yl)carbamoyl)oxy)-2-azaspiro[3.4]octane-2-carboxylate (6b). Yield (67%). ¹H NMR (400 MHz, DMSO-d6) δ 9.40 (d, J=2.0 Hz, 1H), 7.71-7.44 (m, 2H), 7.16 (dt, J=51.0, 7.3 Hz, 1H), 5.01-4.83 (m, 1H), 4.65 (t, J=5.6 Hz, 1H), 4.19 (td, J=7.7, 4.0 Hz, 1H), 4.07-3.84 (m, 1H), 3.87-3.49 (m, 5H), 3.39-3.03 (m, 3H), 2.75-2.70 (m, 1H), 2.32-1.94 (m, 3H), 1.94-1.69 (m, 4H), 1.69-1.57 (m, 3H), 1.37 (s, 9H), 0.95-0.81 (m, 6H). HRMS m/z: [M+Na]⁺ Calculated for C₂₆H₄₂N₄NaO₇ 545.2951; Found 545.2928.

Tert-butyl 2-((((S)-4-methyl-1-oxo-1-(((S)-1-oxo-3-((R)-2-oxopyrrolidin-3-yl)propan-2-yl)amino)pentan-2-yl)carbamoyl)oxy)-7-azaspiro[3.5]nonane-7-carboxylate (7b). Yield (67%). ¹H NMR (500 MHz, DMSO-d6) δ 7.63 (s, 1H), 7.51 (d, J=6.9 Hz, 1H), 7.22-7.16 (m, 1H), 4.86-4.78 (m, 1H), 4.07-3.91 (m, 2H), 3.28-3.03 (m, 6H), 2.30-2.02 (m, 4H), 1.89-1.77 (m, 2H), 1.75-1.50 (m, 4H), 1.49-1.40 (m, 7H), 1.40 (s, 9H), 0.93-0.80 (m, 6H). HRMS m/z: [M+H]⁺ Calculated for C₂₇H₄₅N₄O₇ 537.3288; Found 537.3257.

7-Isobutyryl-7-azaspiro[3.5]nonan-2-yl ((S)-4-methyl-1-oxo-1-(((S)-1-oxo-3-((R)-2-oxopyrrolidin-3-yl)propan-2-yl)amino)pentan-2-yl)carbamate (8b). Yield (55%). ¹H NMR (400 MHz, cdcl₃) δ 9.49 (s, 1H), 8.33 (s, 1H), 6.21-6.13 (m, 1H), 5.29-5.22 (m, 1H), 5.01-4.93 (m, 1H), 4.32 (s, 2H), 3.61-3.45 (m, 2H), 3.44-3.28 (m, 4H), 2.82-2.69 (m, 1H), 2.54-2.27 (m, 5H), 2.12-1.93 (m, 2H), 1.92-1.81 (m, 3H), 1.79-1.63 (m, 1H), 1.56 (s, 5H), 1.10 (d, J=6.7 Hz, 6H), 0.97 (d, J=6.2 Hz, 6H). HRMS m/z: [M+Na]⁺ Calculated for C₂₆H₄₂N₄NaO₆ 529.3002; Found 529.2985.

7-(2-Phenylacetyl)-7-azaspiro[3.5]nonan-2-yl ((S)-4-methyl-1-oxo-1-(((S)-1-oxo-3-((R)-2-oxopyrrolidin-3-yl)propan-2-yl)amino)pentan-2-yl)carbamate (9b). Yield (87%). ¹H NMR (400 MHz, cdcl₃) δ 9.48 (s, 1H), 8.32 (s, 1H), 7.35-7.15 (m, 5H), 6.19 (d, J=13.8 Hz, 1H), 5.28-5.21 (m, 1H), 5.00-4.86 (m, 1H), 4.37-4.10 (m, 2H), 3.72 (s, 2H), 3.59-3.44 (m, 2H), 3.42-3.23 (m, 4H), 2.52-2.34 (m, 2H), 2.34-2.18 (m, 2H), 2.12-1.90 (m, 1H), 1.90-1.76 (m, 3H), 1.74-1.60 (m, 1H), 1.58-1.43 (m, 4H), 1.41-1.32 (m, 3H), 0.96 (d, J=6.1 Hz, 6H). HRMS m/z: [M+H]⁺ Calculated for C₃₀H₄₃N₄O₆ 555.3182; Found 555.3156.

7-(Methylsulfonyl)-7-azaspiro[3.5]nonan-2-yl ((S)-4-methyl-1-oxo-1-(((S)-1-oxo-3-((R)-2-oxopyrrolidin-3-yl)propan-2-yl)amino)pentan-2-yl)carbamate (10b). Yield (62%). ¹H NMR (400 MHz, cdcl₃) δ 9.49 (s, 1H), 8.33 (d, J=5.8 Hz, 1H), 6.10 (s, 1H), 5.25 (d, J=8.6 Hz, 1H), 5.03-4.89 (m, 1H), 4.31 (s, 2H), 3.44-3.29 (m, 2H), 3.21-3.00 (m, 4H), 2.76 (s, 3H), 2.55-2.20 (m, 4H), 2.09-1.80 (m, 4H), 1.69 (td, J=12.3, 7.5 Hz, 7H), 1.54 (t, J=8.8 Hz, 1H), 0.97 (d, J=6.2 Hz, 6H). HRMS m/z: [M+Na]⁺ Calculated for C₂₃H₃₈N₄NaO₇S 537.2359; Found 537.2341. 7-Cyano-7-azaspiro[3.5]nonan-2-yl ((S)-4-methyl-1-oxo-1-(((S)-1-oxo-3-((R)-2-oxopyrrolidin-3-yl)propan-2-yl)amino)pentan-2-yl)carbamate (Jib). Yield (53%). ¹H NMR (400 MHz, cdcl₃) δ 9.49 (s, 1H), 8.36 (d, J=5.7 Hz, 1H), 5.95 (s, 1H), 5.21 (d, J=8.3 Hz, 1H), 5.04-4.89 (m, 1H), 4.38-4.25 (m, 2H), 3.45-3.30 (m, 2H), 3.19-3.08 (m, 4H), 2.56-2.22 (m, 4H), 2.01-1.81 (m, 4H), 1.77-1.62 (m, 7H), 1.61-1.48 (m, 1H), 0.97 (d, J=5.8 Hz, 6H). HRMS m/z: [M+Na]⁺ Calculated for C₂₃H₃₅N₅NaO₅ 484.2536; Found 484.2522.

Tert-butyl 3-((((S)-4-methyl-1-oxo-1-(((S)-1-oxo-3-((R)-2-oxopyrrolidin-3-yl)propan-2-yl)amino)pentan-2-yl)carbamoyl)oxy)azetidine-1-carboxylate (12b). Yield (74%). ¹H NMR (500 MHz, DMSO-d6) δ 7.78 (s, 1H), 7.68-7.61 (m, 1H), 7.54-7.47 (m, 1H), 5.01-4.90 (m, 1H), 4.19-4.05 (m, 2H), 4.05-3.61 (m, 4H), 3.26-3.04 (m, 2H), 2.27-2.02 (m, 3H), 1.86-1.71 (m, 2H), 1.70-1.39 (m, 4H), 1.38-1.34 (m, 9H), 0.92-0.79 (m, 6H). HRMS m/z: [M+Na]⁺ Calculated for C₂₂H₃₆N₄NaO₇ 491.2482; Found 491.2461.

Tert-butyl 3-methyl-3-((((S)-4-methyl-1-oxo-1-(((S)-1-oxo-3-((R)-2-oxopyrrolidin-3-yl)propan-2-yl)amino)pentan-2-yl)carbamoyl)oxy)azetidine-1-carboxylate (13b). Yield (76%). ¹H NMR (400 MHz, DMSO-d6) δ 9.40 (d, J=4.9 Hz, 1H), 8.45 (d, J=7.8 Hz, 1H), 7.63 (s, 1H), 7.50 (d, J=7.7 Hz, 1H), 4.22 (ddd, J=11.6, 7.7, 3.9 Hz, 1H), 4.08-3.93 (m, 1H), 3.88 (d, J=9.3 Hz, 2H), 3.78 (d, J=9.4 Hz, 2H), 3.23-3.02 (m, 2H), 2.34-2.07 (m, 2H), 1.96-1.84 (m, 1H), 1.63 (ddt, J=16.1, 11.8, 6.3 Hz, 3H), 1.55 (s, 3H), 1.46 (qd, J=8.4, 3.9 Hz, 2H), 1.37 (s, 9H), 0.93-0.82 (m, 6H). HRMS m/z: [M+Na]⁺ Calculated for C₂₃H₃₈N₄NaO₇ 505.2638; Found 505.2621.

Tert-butyl 3-(((((S)-4-methyl-1-oxo-1-(((S)-1-oxo-3-((R)-2-oxopyrrolidin-3-yl)propan-2-yl)amino)pentan-2-yl)carbamoyl)oxy)methyl)azetidine-1-carboxylate (14b). Yield (90%). ¹H NMR (400 MHz, DMSO-d6) δ 9.40 (s, 1H), 8.45 (d, J=7.5 Hz, 1H), 7.63 (s, 1H), 7.40 (d, J=7.9 Hz, 1H), 4.21-3.99 (m, 3H), 3.92-3.82 (m, 2H), 3.62-3.52 (m, 2H), 3.21-3.05 (m, 2H), 2.83-2.72 (m, 2H), 2.34-2.06 (m, 2H), 1.95-1.83 (m, 2H), 1.70-1.56 (m, 3H), 1.52-1.42 (m, 1H), 1.37 (s, 9H), 0.92-0.83 (m, 6H). HRMS m/z: [M+Na]⁺ Calculated for C₂₃H₃₈N₄NaO₇ 505.2638; Found 505.2609.

(1-(2-Phenylacetyl)azetidin-3-yl)methyl ((S)-4-methyl-1-oxo-1-(((S)-1-oxo-3-((R)-2-oxopyrrolidin-3-yl)propan-2-yl)amino)pentan-2-yl)carbamate (15b). Yield (63%). ¹H NMR (400 MHz, cdcl₃) δ 9.46 (s, 1H), 8.73-8.66 (m, 1H), 7.40-7.16 (m, 5H), 6.38 (d, J=32.7 Hz, 1H), 6.14 (d, J=22.3 Hz, 1H), 5.32 (d, J=16.0 Hz, 1H), 4.35-3.89 (m, 4H), 3.81-3.65 (m, 2H), 3.60-3.43 (m, 2H), 3.40-3.13 (m, 4H), 2.57-2.19 (m, 2H), 2.06 (s, 1H), 1.98-1.77 (m, 2H), 1.75-1.61 (m, 2H), 1.57-1.45 (m, 1H), 1.03-0.82 (m, 6H). HRMS m/z: [M+Na]⁺ Calculated for C₂₆H₃₆N₄NaO₆ 523.2533; Found 523.2518.

(1-(Bicyclo[2.2.1]heptane-2-carbonyl)azetidin-3-yl)methyl ((S)-4-methyl-1-oxo-1-(((S)-1-oxo-3-((R)-2-oxopyrrolidin-3-yl)propan-2-yl)amino)pentan-2-yl)carbamate (16b). Yield (75%). ¹H NMR (400 MHz, cdcl₃) δ 9.47 (s, 1H), 8.85 (s, 1H), 6.28 (d, J=42.6 Hz, 1H), 6.10 (d, J=32.7 Hz, 1H), 5.32-5.28 (m, 1H), 4.45-3.98 (m, 5H), 3.94-3.70 (m, 1H), 3.69-3.52 (m, 2H), 3.51-3.16 (m, 3H), 2.69-2.56 (m, 1H), 2.56-2.21 (m, 5H), 1.96-1.67 (m, 6H), 1.66-1.46 (m, 2H), 1.43-1.23 (m, 3H), 1.18 (q, J=8.3 Hz, 1H), 0.97 (d, J=5.6 Hz, 6H). HRMS m/z: [M+Na]⁺ Calculated for C₂₆H₄₀N₄NaO₆ 527.2846; Found 527.2837.

(1-(Methylsulfonyl)azetidin-3-yl)methyl ((S)-4-methyl-1-oxo-1-(((S)-1-oxo-3-((R)-2-oxopyrrolidin-3-yl)propan-2-yl)amino)pentan-2-yl)carbamate (17b). Yield (14%). ¹H NMR (400 MHz, cdcl₃) δ 9.48 (s, 1H), 8.28 (d, J=7.5 Hz, 1H), 6.56 (s, 1H), 5.54 (s, 1H), 4.46-3.91 (m, 2H), 3.90-3.73 (m, 2H), 3.70-3.10 (m, 4H), 3.02-2.71 (m, 2H), 2.57-2.16 (m, 3H), 2.16-1.78 (m, 1H), 1.75-1.48 (m, 3H), 1.46-1.36 (m, 2H), 1.26 (s, 3H), 1.08-0.78 (m, 6H). HRMS m/z: [M+Na]⁺ Calculated for C₁₉H₃₂N₄NaO₇S 483.1890; Found 483.1832.

Preparation of compounds 1-17c. General procedure. To a solution of dipeptidyl aldehyde b (1 eq) in ethyl acetate (10 mL/g of dipeptidyl aldehyde) was added absolute ethanol (5 mL/g of dipeptidyl aldehyde) with stirring, followed by a solution of sodium bisulfite (1 eq) in water (1 mL/g of dipeptidyl aldehyde). The reaction mixture was stirred for 3 h at 50° C. The reaction mixture was allowed to cool to room temperature and then vacuum filtered. The solid was thoroughly washed with absolute ethanol and the filtrate was dried over anhydrous sodium sulfate, filtered, and concentrated to yield a white solid. The white solid was stirred with dry ethyl ether (3×10 mL/g of dipeptidyl aldehyde), followed by careful removal of the solvent using a pipette and dried using a vacuum pump for 2 h to yield dipeptidyl bisulfite adduct c as a white solid.

Sodium (2S)-2-((S)-2-((((2-(tert-butoxycarbonyl)-2-azaspiro[3.3]heptan-6-yl)oxy) carbonyl)amino)-4-methylpentanamido)-1-hydroxy-3-((R)-2-oxopyrrolidin-3-yl)propane-1-sulfonate (1c). Yield (56%). ¹H NMR (400 MHz, DMSO-d6) δ 7.52 (d, J=9.3 Hz, 1H), 7.44 (s, 1H), 7.18 (d, J=8.2 Hz, 1H), 5.71 (d, J=5.9 Hz, 1H), 4.74-4.59 (m, 2H), 4.08-3.58 (m, 5H), 3.23-2.99 (m, 2H), 2.29-1.94 (m, 4H), 1.91-1.71 (m, 1H), 1.69-1.38 (m, 7H), 1.35 (s, 9H), 0.91-0.79 (m, 6H). HRMS m/z: [M+Na]⁺ Calculated for C₂₅H₄₁N₄Na₂O₁₀S 635.2339; Found 635.2379.

Sodium (2S)-1-hydroxy-2-((S)-2-((((2-isobutyryl-2-azaspiro[3.3]heptan-6-yl)oxy) carbonyl)amino)-4-methylpentanamido)-3-((R)-2-oxopyrrolidin-3-yl)propane-1-sulfonate (2c). Yield (69%). ¹H NMR (400 MHz, DMSO-d₆) δ 7.85 (s, 1H), 7.64 (s, 1H), 7.19 (s, 1H), 5.80-5.66 (m, 1H), 4.90-4.60 (m, 2H), 4.28-3.81 (m, 2H), 3.81-3.59 (m, 2H), 3.25-2.98 (m, 4H), 2.47-2.34 (m, 1H), 2.34-1.75 (m, 7H), 1.75-1.29 (m, 4H), 1.01 (d, J=6.8 Hz, 6H), 0.95-0.77 (m, 6H). HRMS m/z: [M+H]⁺ Calculated for C₂₄H₄₀N₄NaO₉S 583.2413; Found 583.2675.

Sodium (2S)-1-hydroxy-2-((S)-4-methyl-2-((((2-(2-phenylacetyl)-2-azaspiro[3.3]heptan-6-yl)oxy)carbonyl)amino)pentanamido)-3-((R)-2-oxopyrrolidin-3-yl)propane-1-sulfonate (3c). Yield (97%). ¹H NMR (400 MHz, DMSO-d6) δ 8.41-8.32 (m, 1H), 8.21 (dd, J=13.7, 7.3 Hz, 1H), 7.47 (d, J=3.9 Hz, 1H), 7.35-7.14 (m, 5H), 5.55 (dd, J=188.2, 6.3 Hz, 1H), 4.86-4.70 (m, 1H), 4.07-3.87 (m, 2H), 3.86-3.54 (m, 2H), 3.49-3.42 (m, 2H), 3.42-3.31 (m, 4H), 3.29-2.99 (m, 2H), 2.32-1.85 (m, 6H), 1.70-1.49 (m, 2H), 1.49-1.39 (m, 2H), 0.93-0.79 (m, 6H). HRMS m/z: [M+H]⁺ Calculated for C₂₈H₄₀N₄NaO₉S 631.2853; Found 631.2413.

Sodium (2S)-1-hydroxy-2-((S)-4-methyl-2-((((2-(methylsulfonyl)-2-azaspiro[3.3]heptan-6-yl)oxy)carbonyl)amino)pentanamido)-3-((R)-2-oxopyrrolidin-3-yl)propane-1-sulfonate (4c). Yield (90%). ¹H NMR (400 MHz, DMSO-d6) δ 7.63 (s, 1H), 7.37 (d, J=8.0 Hz, 1H), 7.19-7.15 (m, 1H), 5.73-5.67 (m, 1H), 5.01-4.78 (m, 2H), 4.78-4.59 (m, 1H), 4.09-3.67 (m, 6H), 3.23-2.98 (m, 4H), 2.91 (s, 3H), 2.38-2.06 (m, 4H), 2.06-1.76 (m, 2H), 1.73-1.53 (m, 1H), 1.53-1.33 (m, 1H), 0.98-0.78 (m, 6H). HRMS m/z: [M+H]⁺ Calculated for C₂₁H₃₆N₄NaO₁₀S₂ 591.1770; Found 591.1647.

Sodium (2S)-2-((S)-2-((((6-(tert-butoxycarbonyl)-6-azaspiro[3.4]octan-2-yl)oxy)carbonyl)amino)-4-methylpentanamido)-1-hydroxy-3-((R)-2-oxopyrrolidin-3-yl)propane-1-sulfonate (5c). Yield (22%). ¹H NMR (400 MHz, DMSO-d6) δ 7.63 (s, 1H), 7.53 (s, 1H), 7.24-7.20 (m, 1H), 5.73-5.68 (m, 1H), 4.86-4.77 (m, 1H), 4.07-3.77 (m, 2H), 3.67-3.38 (m, 4H), 3.28-2.95 (m, 6H), 2.37-2.20 (m, 2H), 2.20-2.05 (m, 1H), 2.05-1.88 (m, 2H), 1.88-1.74 (m, 3H), 1.74-1.45 (m, 2H), 1.39 (s, 9H), 0.92-0.81 (m, 6H). HRMS m/z: [M+Na]⁺ Calculated for C₂₆H₄₃N₄Na₂O₁₀S 649.2496; Found 649.2458.

Sodium (2S)-2-((2S)-2-((((2-(tert-butoxycarbonyl)-2-azaspiro[3.4]octan-6-yl)oxy)carbonyl)amino)-4-methylpentanamido)-1-hydroxy-3-((R)-2-oxopyrrolidin-3-yl)propane-1-sulfonate (6c). Yield (7%). ¹H NMR (400 MHz, DMSO-d6) δ 7.63-7.38 (m, 2H), 7.30-7.03 (m, 1H), 5.30 (dt, J=54.1, 5.9 Hz, 1H), 5.00-4.81 (m, 1H), 4.66 (t, J=5.6 Hz, 1H), 4.02-3.86 (m, 2H), 3.83-3.48 (m, 4H), 3.37-2.97 (m, 3H), 2.29-1.96 (m, 3H), 1.96-1.67 (m, 5H), 1.67-1.48 (m, 4H), 1.37 (s, 9H), 0.94-0.77 (m, 6H). HRMS m/z: [M+Na]⁺ Calculated for C₂₆H₄₃N₄Na₂O₁₀S 649.2496; Found 649.2454.

Sodium (2S)-2-((S)-2-((((7-(tert-butoxycarbonyl)-7-azaspiro[3.5]nonan-2-yl)oxy) carbonyl)amino)-4-methylpentanamido)-1-hydroxy-3-((R)-2-oxopyrrolidin-3-yl)propane-1-sulfonate (7c). Yield (87%). ¹H NMR (400 MHz, DMSO-d6) δ 7.57-7.50 (m, 1H), 7.45 (s, 1H), 7.28 (dd, J=35.4, 8.4 Hz, 1H), 5.33 (dd, J=56.7, 6.1 Hz, 1H), 4.88-4.77 (m, 2H), 4.42-4.10 (m, 1H), 4.07-3.76 (m, 4H), 3.27-3.00 (m, 6H), 2.36-1.85 (m, 4H), 1.85-1.66 (m, 1H), 1.65-1.50 (m, 1H), 1.43 (d, J=14.3 Hz, 6H), 1.38 (s, 9H), 0.89-0.79 (m, 6H). HRMS m/z: [M+Na]⁺ Calculated for C₂₇H₄₅N₄Na₂O₁₀S 663.2652; Found 663.2690.

Sodium (2S)-1-hydroxy-2-((S)-2-((((7-isobutyryl-7-azaspiro[3.5]nonan-2-yl)oxy) carbonyl)amino)-4-methylpentanamido)-3-((R)-2-oxopyrrolidin-3-yl)propane-1-sulfonate (8c). Yield (80%). ¹H NMR (400 MHz, DMSO-d6) δ 7.63 (s, 1H), 7.46 (s, 1H), 7.36-7.28 (m, 1H), 5.42 (dd, J=64.4, 6.1 Hz, 1H), 4.87-4.81 (m, 1H), 4.52-4.12 (m, 2H), 4.09-3.80 (m, 2H), 3.22-2.97 (m, 4H), 2.88-2.79 (m, 2H), 2.37-2.18 (m, 3H), 2.18-1.96 (m, 1H), 1.96-1.68 (m, 3H), 1.68-1.32 (m, 8H), 1.00-0.94 (m, 6H), 0.92-0.80 (m, 6H). HRMS m/z: [M+Na]⁺ Calculated for C₂₆H₄₃N₄Na₂O₉S 633.2546; Found 633.2526.

Sodium (2S)-1-hydroxy-2-((S)-4-methyl-2-((((7-(2-phenylacetyl)-7-azaspiro[3.5]nonan-2-yl)oxy)carbonyl)amino)pentanamido)-3-((R)-2-oxopyrrolidin-3-yl)propane-1-sulfonate (9c). Yield (68%). ¹H NMR (400 MHz, DMSO-d6) δ 7.60-7.49 (m, 2H), 7.45 (s, 1H), 7.35-7.11 (m, 5H), 5.38 (dd, J=60.0, 6.1 Hz, 1H), 4.86-4.73 (m, 2H), 4.44-4.12 (m, 1H), 4.06-3.77 (m, 4H), 3.71-3.61 (m, 4H), 3.22-2.99 (m, 2H), 2.35-2.03 (m, 4H), 2.03-1.79 (m, 1H), 1.78-1.65 (m, 1H), 1.63-1.49 (m, 1H), 1.48-1.27 (m, 7H), 0.91-0.79 (m, 6H). HRMS m/z: [M+Na]⁺ Calculated for C₃₀H₄₃N₄Na₂O₉S 681.2546; Found 681.2522.

Sodium (2S)-1-hydroxy-2-((S)-4-methyl-2-((((7-(methylsulfonyl)-7-azaspiro[3.5]nonan-2-yl)oxy)carbonyl)amino)pentanamido)-3-((R)-2-oxopyrrolidin-3-yl)propane-1-sulfonate (10c). Yield (71%). ¹H NMR (400 MHz, DMSO-d6) δ 7.62 (d, J=9.3 Hz, 1H), 7.45 (s, 1H), 7.38-7.31 (m, 1H), 5.41 (dd, J=73.2, 6.1 Hz, 1H), 4.88-4.76 (m, 1H), 4.28-3.76 (m, 2H), 3.21-2.91 (m, 6H), 2.83 (s, 3H), 2.35-1.98 (m, 3H), 1.96-1.68 (m, 4H), 1.67-1.50 (m, 6H), 1.49-1.32 (m, 2H), 1.14-1.01 (m, 1H), 0.91-0.78 (m, 6H). HRMS m/z: [M+Na]⁺ Calculated for C₂₃H₃₉N₄Na₂O₁₀S₂ 641.1903; Found 641.1874.

Sodium (2S)-2-((S)-2-((((7-cyano-7-azaspiro[3.5]nonan-2-yl)oxy)carbonyl)amino)-4-methylpentanamido)-1-hydroxy-3-((R)-2-oxopyrrolidin-3-yl)propane-1-sulfonate (11c). Yield (74%). ¹H NMR (400 MHz, DMSO-d6) δ 8.43 (d, J=7.6 Hz, 1H), 7.63 (s, 1H), 7.30 (d, J=8.0 Hz, 1H), 4.86-4.76 (m, 1H), 4.26-4.08 (m, 1H), 4.06-3.80 (m, 1H), 3.40-3.24 (m, 2H), 3.22-3.00 (m, 4H), 2.36-2.02 (m, 4H), 1.95-1.63 (m, 2H), 1.62-1.49 (m, 7H), 1.49-1.30 (m, 2H), 1.15-1.02 (m, 2H), 0.96-0.76 (m, 6H). HRMS m/z: [M+H]⁺ Calculated for C₂₃H₃₇N₅NaO₈S 566.2260; Found 566.2238.

Sodium (2S)-2-((S)-2-((((1-(tert-butoxycarbonyl)azetidin-3-yl)oxy)carbonyl)amino)-4-methylpentanamido)-1-hydroxy-3-((R)-2-oxopyrrolidin-3-yl)propane-1-sulfonate (12c). Yield (64%). ¹H NMR (400 MHz, DMSO-d6) δ 7.66 (d, J=11.1 Hz, 2H), 7.58-7.42 (m, 1H), 5.01-4.90 (m, 2H), 4.71-4.64 (m, 1H), 4.23-3.84 (m, 3H), 3.84-3.51 (m, 2H), 3.19-3.04 (m, 2H), 2.34-2.01 (m, 2H), 2.00-1.73 (m, 1H), 1.71-1.43 (m, 5H), 1.38 (s, 9H), 0.92-0.81 (m, 6H). HRMS m/z: [M+Na]⁺ Calculated for C₂₂H₃₇N₄Na₂O₁₀S 595.2026; Found 595.1995.

Sodium (2S)-2-((S)-2-((((1-(tert-butoxycarbonyl)-3-methylazetidin-3-yl)oxy)carbonyl) amino)-4-methylpentanamido)-1-hydroxy-3-((R)-2-oxopyrrolidin-3-yl)propane-1-sulfonate (13c). Yield (33%). ¹H NMR (400 MHz, DMSO-d6) δ 7.64 (d, J=10.0 Hz, 1H), 7.58-7.35 (m, 2H), 4.29-4.10 (m, 1H), 4.08-3.86 (m, 3H), 3.77-3.69 (m, 3H), 3.18-2.98 (m, 2H), 2.37-2.04 (m, 2H), 2.02-1.77 (m, 1H), 1.77-1.50 (m, 6H), 1.48-1.34 (m, 11H), 0.93-0.80 (m, 6H). HRMS m/z: [M+Na]⁺ Calculated for C₂₃H₃₉N₄Na₂O₁₀S 609.2183; Found 609.2160.

Sodium (2S)-2-((S)-2-((((1-(tert-butoxycarbonyl)azetidin-3-yl)methoxy)carbonyl)amino)-4-methylpentanamido)-1-hydroxy-3-((R)-2-oxopyrrolidin-3-yl)propane-1-sulfonate (14c). Yield (57%). ¹H NMR (400 MHz, DMSO-d6) δ 7.59 (dd, J=9.2, 5.5 Hz, 1H), 7.43 (s, 1H), 7.36-7.23 (m, 1H), 5.34 (dd, J=69.8, 6.1 Hz, 1H), 4.14-4.01 (m, 2H), 4.01-3.76 (m, 3H), 3.62-3.47 (m, 2H), 3.20-2.98 (m, 3H), 2.87-2.67 (m, 1H), 2.24-2.06 (m, 3H), 2.04-1.80 (m, 1H), 1.72-1.48 (m, 3H), 1.46-1.39 (m, 1H), 1.37 (s, 9H), 0.92-0.80 (m, 6H). HRMS m/z: [M+Na]⁺ Calculated for C₂₃H₃₉N₄Na₂O₁₀S 609.2183; Found 609.2205.

Sodium (2S)-1-hydroxy-2-((S)-4-methyl-2-((((1-(2-phenylacetyl)azetidin-3-yl)methoxy) carbonyl)amino)pentanamido)-3-((R)-2-oxopyrrolidin-3-yl)propane-1-sulfonate (15c). Yield (92%). ¹H NMR (400 MHz, DMSO-d6) δ 8.16 (s, 1H), 7.64 (s, 1H), 7.52-7.44 (m, 1H), 7.34-7.14 (m, 5H), 4.22 (d, J=6.5 Hz, 2H), 4.14-3.79 (m, 4H), 3.72-3.54 (m, 2H), 3.50-3.38 (m, 2H), 3.23-3.00 (m, 4H), 2.38-1.95 (m, 3H), 1.93-1.72 (m, 1H), 1.72-1.53 (m, 2H), 1.53-1.30 (m, 2H), 0.92-0.80 (m, 6H). HRMS m/z: [M+H]⁺ Calculated for C₂₆H₃₈N₄NaO₉S 605.2257; Found 605.2698.

Sodium (2S)-2-((2S)-2-((((1-(bicyclo[2.2.1]heptane-2-carbonyl)azetidin-3-yl)methoxy) carbonyl)amino)-4-methylpentanamido)-1-hydroxy-3-((R)-2-oxopyrrolidin-3-yl)propane-1-sulfonate (16c). Yield (71%). ¹H NMR (400 MHz, DMSO-d6) δ 7.78 (s, 1H), 7.64 (s, 1H), 7.38 (s, 1H), 4.18 (s, 1H), 4.13-3.79 (m, 3H), 3.72-3.54 (m, 2H), 3.26-2.96 (m, 4H), 2.70-2.55 (m, 1H), 2.36-2.02 (m, 4H), 2.00-1.76 (m, 1H), 1.75-1.32 (m, 9H), 1.29-1.19 (m, 4H), 1.19-1.00 (m, 2H), 0.93-0.77 (m, 6H). HRMS m/z: [M+H]⁺ Calculated for C₂₆H₄₂N₄NaO₉S 609.2570; Found 609.3013.

Sodium (2S)-1-hydroxy-2-((S)-4-methyl-2-((((1-(methylsulfonyl)azetidin-3-yl)methoxy) carbonyl)amino)pentanamido)-3-((R)-2-oxopyrrolidin-3-yl)propane-1-sulfonate (17c). Yield (88%). ¹H NMR (400 MHz, DMSO-d6) δ 7.64 (s, 1H), 7.16 (s, 1H), 6.96 (s, 1H), 4.67 (s, 2H), 4.29-3.83 (m, 5H), 3.81-3.52 (m, 3H), 3.24-2.96 (m, 2H), 2.36-2.02 (m, 2H), 1.95-1.73 (m, 1H), 1.59 (s, 4H), 1.37 (s, 3H), 1.24 (s, 1H), 0.97-0.77 (m, 6H). HRMS m/z: [M+H]⁺ Calculated for C₁₉H₃₄N₄NaO₁₀S₂ 565.1614; Found 565.1878.

### Biochemical Studies

Enzyme assays and inhibition studies. Cloning and expression of the 3CL protease of SARS-CoV-2 and FRET enzyme assays. The codon-optimized cDNA of full length of 3CLpro of SARS-CoV-2 (GenBank number MN908947.3) fused with sequences encoding 6 histidine at the N-terminal was synthesized by Integrated DNA (Coralville, IA). The synthesized gene was subcloned into the pET-28a(+) vector. The expression and purification of SARS-CoV-2 3CLpro were conducted following a standard procedure. Briefly, a stock solution of an inhibitor was prepared in DMSO and diluted in assay buffer comprised of 20 mM HEPES buffer, pH 8, containing NaCl (200 mM), EDTA (0.4 mM), glycerol (60%), and 6 mM dithiothreitol (DTT). The SARS-CoV-2 protease was mixed with serial dilutions of inhibitors 1-17b/c or with DMSO in 25 μL of assay buffer and incubated at 37° C. for 1 h, followed by the addition of 25 μL of assay buffer containing substrate (FAM-SAVLQ/SG-QXL®520, AnaSpec, Fremont, CA). The substrate was derived from the cleavage sites on the viral polyproteins of SARS-CoV. Fluorescence readings were obtained using an excitation wavelength of 480 nm and an emission wavelength of 520 nm on a fluorescence microplate reader (FLx800; Biotec, Winoosk, VT) 1 h following the addition of substrate. Relative fluorescence units (RFU) were determined by subtracting background values (substrate-containing well without protease) from the raw fluorescence values. The dose-dependent FRET inhibition curves were fitted with a variable slope by using GraphPad Prism software (GraphPad, La Jolla, CA) in order to determine the IC₅₀ values of the compounds.

Antiviral Assays/Cell-based inhibition assays. To assess antiviral effects of selected compounds (dissolved in DMSO) in cell culture, the SARS-CoV-2 replicon system with pSMART-T7-scv2-replicon (pSMART® BAC V2.0 Vector Containing the SARS-CoV-2, Wuhan-Hu-1 Non-Infectious Replicon) was used. The synthetic SARS-CoV-2 replicon RNA was prepared from the pSMART-T7-scv2-replicon, and the Neon Electroporation system (ThermoFisher, Chicago, IL) was used for the RNA electroporation to 293T cells. After the electroporation, cells were incubated with DMSO (0.1%) or each compound at 2, 0.5, 0.1 and 0.02 uM for 30 hr, and luciferase activities were measured for antiviral effects. The dose-dependent inhibition curve for each compound was prepared and the 50% effective concentration (EC₅₀) values were determined by GraphPad Prism software using a variable slope (GraphPad, La Jolla, CA).

Nonspecific cytotoxic effects/Measurement of in vitro cytotoxicity. Confluent cells grown in 96-well plates were incubated with various concentrations (1 to 100 μM) of each compound for 72 h. Cell cytotoxicity was measured by a CytoTox 96 nonradioactive cytotoxicity assay kit (Promega, Madison, WI), and the CC₅₀ values were calculated using a variable slope by GraphPad Prism software. The in vitro Safety Index was calculated by dividing the CC₅₀ by the EC₅₀.

### X-Ray Crystallographic Studies

Crystallization and Data Collection. Purified MERS-CoV 3CL^{pro} and SARS-CoV-2 3CL^{pro} in 100 mM NaCl, 20 mM Tris pH 8.0 were concentrated to 10 mg/mL (0.3 mM) for crystallization screening. Stock solutions of the inhibitors were prepared in DMSO at 100 mM and the complexes with the 3CL proteases were prepared by adding 2 mM of each compound and incubating the complexes on ice for 1 hour. All crystallization experiments were setup using an NT8 drop-setting robot (Formulatrix Inc.) and UVXPO MRC (Molecular Dimensions) sitting drop vapor diffusion plates at 18° C. 100 nL of protein and 100 nL crystallization solution were dispensed and equilibrated against 50 uL of the latter. Crystals of the MERS-CoV 3CL^{pro} complexes were obtained from the following conditions. Index HT screen (Hampton Research) 9c: condition E7 (30% (w/v) PEG 550 MME, 100 mM Hepes pH 7.5, 50 mM magnesium chloride), 8c: condition F7 (20% (w/v) PEG 3350, 100 mM Bis-Tris pH 6.5, 200 mM ammonium sulfate) and 10c: condition F5 (17% (w/v) PEG 10000, 100 mM Bis-Tris pH 5.5, 100 mM ammonium acetate). Proplex HT screen (Molecular Dimensions) 14c: condition E2 (25% (w/v) PEG 3350, 100 mM Hepes pH 7.5, 200 mM magnesium chloride). Crystals of the SARS-CoV-2 3CL^{pro} complexes were obtained from the following conditions. PACT screen (Molecular Dimensions) 2c: condition C2 (25% (w/v) PEG 1500, 100 mM PCTP pH 5.0), 3c: condition C1 (25% (w/v) PEG 1500, 100 mM PCTP pH 4.0), 11c: condition E1 (20% (w/v) PEG 3350, 20 mM sodium/potassium phosphate) and 10c: condition D4 (25% (w/v) PEG 1500, 100 MMT pH 7.0), Index HT screen (Hampton Research) 4c: condition F5 (17% (w/v) PEG 10000, 100 mM Bis-Tris pH 5.5, 100 mM ammonium acetate), 8c: condition F10 (25% (w/v) PEG 3350, 100 mM Bis-Tris pH 5.5, 200 mM NaCl), 14c: condition F11 (25% (w/v) PEG 3350, 100 mM Bis-Tris pH 6.5, 200 mM sodium chloride), 9c: condition G4 (20% (w/v) PEG 3350, 100 mM Hepes pH 7.5, 200 mM lithium sulfate) and Berkeley screen (Rigaku Reagents) 7c: condition B6 (20% (w/v) PEG 3350, 200 mM sodium fluoride). Cryoprotectants containing 80% crystallant and 20% (v/v) PEG 200 were layered onto the drop, samples were harvested and stored in liquid nitrogen. For MERS-CoV 3CL^{pro} in complex with 9c, the crystallization solution served as the cryoprotectant. X-ray diffraction data were collected at the Advanced Photon Source beamline 17-ID (IMCA-CAT) and National Synchrotron Light Source-II, beamline 19-ID (NYX).

Structure Solution and Refinement. Intensities were integrated using XDS via Autoproc and the Laue class analysis and data scaling were performed with Aimless. Structure solution was conducted by molecular replacement with Phaser using a previously determined inhibitor bound structures of MERS-CoV (SWKK) and SARS-CoV-2 3CL^{pro} (PDB 6XM1K) as the search models. Structure refinement and manual model building were conducted with Phenix and Coot, respectively. Disordered side chains were truncated to the point for which electron density could be observed. Structure validation was conducted with Molprobity and figures were prepared using the CCP4MG package. Crystallographic data are provided in Tables 13 and 14 below.

TABLE 13
Crystallographic data for MERS 3CLpro.

| | Compound Code | | | | | | | |
|---|---|---|---|---|---|---|---|---|
| | 8c | | 9c | | 10c | | 15c | |
| | PDB Code | | | | | | | |
| | 7T3Y | | 7T3Z | | 7T40 | | 7T41 | |
| Data Collection | | | | | | | | |
| Unit-cell parameters | a = 75.23 | | a = 49.66 | | a = 101.61 | | a = 101.34 | |
| (Å, °) | b = 91.33 | | b = 106.13, | | b = 58.06 | | b = 57.87 | |
| | c = 99.17 | | c = 58.70 | | c = 49.90 | | c = 49.73 | |
| | | | β = 108.9 | | β = 112.7 | | β = 112.1 | |
| Space group | P2₁2₁2₁ | | P2₁ | | C2 | | C2 | |
| Resolution (Å)¹ | 45.66-1.90 | | 46.99-1.95 | | 46.87-1.70 | | 46.96-2.10 | |
| | (1.94-1.90) | | (2.00-1.95) | | (1.73-1.70) | | (2.16-2.10) | |
| Wavelength (Å) | 1.0000 | | 1.0000 | | 1.0000 | | 0.9795 | |
| Temperature (K) | 100 | | 100 | | 100 | | 100 | |
| Observed reflections | 369,090 | | 268,646 | | 100,588 | | 50,969 | |
| Unique reflections | 54,463 | | 40,703 | | 29,415 | | 15,305 | |
| <I/σ(I)>¹ | 14.8 | (1.4) | 10.2 | (2.4) | 10.5 | (1.7) | 7.9 | (2.0) |
| Completeness (%)¹ | 99.9 | (99.6) | 97.7 | (97.7) | 99.5 | (99.7) | 97.6 | (99.3) |
| Multiplicity¹ | 6.8 | (7.0) | 6.6 | (6.8) | 3.4 | (3.1) | 3.3 | (3.4) |
| R_{merge} (%) ^{1, 2} | 6.0 | (175.0) | 11.3 | (80.9) | 6.5 | (67.3) | 12.2 | (72.0) |
| Rₘₑₐₛ (%) ^{1, 4} | 6.5 | (188.7) | 12.3 | (87.4) | 7.7 | (81.9) | 14.5 | (85.5) |
| Rₚᵢₘ (%) ^{1, 4} | 2.5 | (70.2) | 4.7 | (32.8) | 4.1 | (46.1) | 7.8 | (45.5) |
| CC_{1/2} ^{1, 5} | 0.999 | (0.723) | 0.997 | (0.776) | 0.998 | (0.749) | 0.993 | (0.599) |

| | | | | |
|---|---|---|---|---|
| Refinement | | | | |
| Resolution (Å) ¹ | 45.66-1.90 | 46.99-1.95 | 46.87-1.70 | 37.55-2.10 |
| Reflections | 51,603/2,736 | 38,631/2,039 | 27,953/1,448 | 14,602/698 |
| (working/test)¹ | | | | |
| R_{factor}/R_{free} (%)^{1, 3} | 20.5/25.8 | 15.7/20.6 | 15.6/19.5 | 16.5/23.7 |
| No. of atoms | 4,484/144/189 | 4,581/120/342 | 2,276/70/232 | 2,233/68/78 |
| (Protein/Ligand/Water) | | | | |
| Model Quality | | | | |
| R.m.s deviations | | | | |
| Bond lengths (Å) | 0.012 | 0.011 | 0.011 | 0.011 |
| Bond angles (°) | 1.121 | 1.071 | 0.995 | 1.115 |
| Average B-factor (Å²) | | | | |
| All Atoms | 51.2 | 30.5 | 25.2 | 33.6 |
| Protein | 51.1 | 30.0 | 24.4 | 33.3 |
| Ligand | 58.9 | 32.7 | 22.2 | 40.1 |
| Water | 48.7 | 35.9 | 33.2 | 34.6 |
| Coordinate | 0.29 | 0.21 | 0.18 | 0.26 |
| error(maximum | | | | |
| likelihood) (Å) | | | | |
| Ramachandran Plot | | | | |
| Most favored (%) | 98.2 | 98.5 | 99.3 | 97.7 |
| Additionally allowed | 1.8 | 1.5 | 0.7 | 1.7 |
| (%) | | | | |

Values in parenthesis are for the highest resolution shell.
R_{merge} = ΣₕₖₗΣᵢ |Iᵢ(hkl) − <I(hkl)>|/ΣₕₖₗΣᵢ Iᵢ(hkl), where Iᵢ(hkl) is the intensity measured for the ith reflection and <I(hkl)> is the average intensity of all reflections with indices hkl.
R_{factor} = Σₕₖₗ ||F_{obs} (hkl) | − |F_{calc} (hkl) ||/Σₕₖₗ |F_{obs} (hkl)|; R_{Free} is calculated in an identical manner using 5% of randomly selected reflections that were not included in the refinement.
Rₘₑₐₛ = redundancy-independent (multiplicity-weighted) R_{merge}. Rₚᵢₘ = precision−indicating (multiplicity-weighted) R_{merge}.
CC_{1/2} is the correlation coefficient of the mean intensities between two random half-sets of data.

TABLE 14
Crystallographic data for SARS-CoV-2 3CLpro.

| | Compound Code | | | | | | | |
|---|---|---|---|---|---|---|---|---|
| | 2c | | 3c | | 4c | | 7c | |
| | PDB Code | | | | | | | |
| | 7T42 | | 7T43 | | 7T44 | | 7T45 | |
| Data Collection | | | | | | | | |
| Unit-cell parameters | a = 55.10 | | a = 55.29 | | a = 55.55 | | a = 55.30 | |
| (Å, °) | b = 98.57 | | b = 98.84 | | b = 98.75 | | b = 98.72 | |
| | c = 58.69 | | c = 58.86 | | c = 59.32 | | c = 58.90 | |
| | β = 107.7 | | β = 107.9 | | β = 108.3 | | β = 107.9 | |
| Space group | P2₁ | | P2₁ | | P2₁ | | P2₁ | |
| Resolution (Å)¹ | 49.28-1.60 | | 48.74-1.70 | | 49.38-1.45 | | 49.36-1.65 | |
| | (1.63-1.60) | | (1.73-1.70) | | (1.47-1.45) | | (1.68-1.65) | |
| Wavelength (Å) | 0.9795 | | 0.9795 | | 0.9795 | | 1.0000 | |
| Temperature (K) | 100 | | 100 | | 100 | | 100 | |
| Observed reflections | 532,226 | | 213,050 | | 364,972 | | 246,835 | |
| Unique reflections | 78,397 | | 64,486 | | 105,771 | | 72,006 | |
| <I/σ(I)>¹ | 9.7 | (1.7) | 9.9 | (2.0) | 10.0 | (1.9) | 11.1 | (1.4) |
| Completeness (%)¹ | 99.7 | (99.6) | 97.6 | (98.7) | 98.7 | (96.4) | 99.6 | (97.8) |
| Multiplicity¹ | 6.8 | (6.9) | 3.3 | (3.3) | 3.5 | (3.1) | 3.4 | (2.7) |
| R_{merge} (%) ^{1, 2} | 8.8 | (111.0) | 4.9 | (53.5) | 6.5 | (58.6) | 5.3 | (69.6) |
| Rₘₑₐₛ (%) ^{1, 4} | 9.5 | (119.8) | 5.8 | (63.8) | 7.7 | (70.5) | 6.2 | (87.1) |
| Rₚᵢₘ (%) ^{1, 4} | 3.6 | (44.8) | 3.2 | (34.4) | 4.1 | (38.8) | 3.3 | (51.6) |
| CC_{1/2} ^{1, 5} | 0.998 | (0.796) | 0.999 | (0.831) | 0.995 | (0.746) | 0.998 | (0.592) |
| Refinement | | | | | | | | |
| Resolution (Å) ¹ | 36.97-1.60 | | 48.74-1.70 | | 37.12-1.45 | | 36.02-1.65 | |
| Reflections | 74,621/3,681 | | 61,337/3,104 | | 100,574/5,153 | | 68,413/3,534 | |
| (working/test)¹ | | | | | | | | |
| R_{factor}/R_{free} (%)^{1, 3} | 18.0/21.2 | | 17.7/21.3 | | 19.5/22.6 | | 18.5/22.1 | |
| No. of atoms | 4,563/122/257 | | 4,585/117/217 | | 4,598/116/358 | | 4,472/64/334 | |
| (Protein/Ligand/Water) | | | | | | | | |
| Model Quality | | | | | | | | |
| R.m.s deviations | | | | | | | | |
| Bond lengths (Å) | 0.008 | | 0.007 | | 0.004 | | 0.011 | |
| Bond angles (°) | 0.939 | | 0.878 | | 0.727 | | 1.050 | |
| Average B-factor (Å²) | | | | | | | | |
| All Atoms | 29.8 | | 30.6 | | 24.9 | | 32.8 | |
| Protein | 29.5 | | 30.4 | | 24.5 | | 32.2 | |
| Ligand | 28.8 | | 30.4 | | 25.1 | | 37.4 | |
| Water | 34.9 | | 34.6 | | 31.0 | | 37.6 | |
| Coordinate | 0.18 | | 0.17 | | 0.17 | | 0.22 | |
| error(maximum | | | | | | | | |
| likelihood) (Å) | | | | | | | | |
| Ramachandran Plot | | | | | | | | |
| Most favored (%) | 98.2 | | 98.5 | | 98.5 | | 96.5 | |
| Additionally allowed | 1.8 | | 1.5 | | 1.5 | | 3.5 | |
| (%) | | | | | | | | |
| | Compound Code | | | | | | | |
| | 9c | | 10c | | 11c | | 15c | |
| | PDB Code | | | | | | | |
| | 7T48 | | 7T49 | | 7T4A | | 7T4B | |
| Data Collection | | | | | | | | |
| Unit-cell parameters | a = 113.66 | | a = 55.31 | | a = 55.56 | | a = 55.42 | |
| (Å, °) | b = 52.59 | | b = 98.33 | | b = 98.81 | | b = 99.03 | |
| | c = 46.31 | | c = 58.79 | | c = 58.81 | | c = 59.11 | |
| | β = 102.9 | | b = 108.1 | | β = 107.7 | | β = 108.2 | |
| Space group | C2 | | P2₁ | | P2₁ | | P2₁ | |
| Resolution (Å)¹ | 47.51-1.90 | | 49.16-1.75 | | 48.73-1.80 | | 48.85-1.60 | |
| | (1.94-1.90) | | (1.78-1.75) | | (1.84-1.80) | | (1.63-1.60) | |
| Wavelength (Å) | 1.0000 | | 1.0000 | | 1.0000 | | 0.9795 | |
| Temperature (K) | 100 | | 100 | | 100 | | 100 | |
| Observed reflections | 70,653 | | 201,886 | | 192,466 | | 269,373 | |
| Unique reflections | 20,929 | | 59,451 | | 55,643 | | 79,076 | |
| <I/σ(I)>¹ | 8.4 | (2.0) | 10.1 | (1.8) | 12.7 | (1.7) | 11.5 | (1.7) |
| Completeness (%)¹ | 99.0 | (96.9) | 98.9 | (99.3) | 99.4 | (99.9) | 99.2 | (99.9) |
| Multiplicity¹ | 3.4 | (3.3) | 3.4 | (3.4) | 3.5 | (3.3) | 3.4 | (3.5) |
| R_{merge} (%) ^{1, 2} | 9.6 | (88.1) | 5.9 | (66.9) | 5.0 | (75.2) | 5.8 | (82.0) |
| Rₘₑₐₛ (%) ^{1, 4} | 11.4 | (106.2) | 7.0 | (79.2) | 5.9 | (87.5) | 6.9 | (97.1) |
| Rₚᵢₘ (%) ^{1, 4} | 6.1 | (58.3) | 3.7 | (42.2) | 3.1 | (49.0) | 3.7 | (51.4) |
| CC_{1/2} ^{1, 5} | 0.993 | (0.682) | 0.998 | (0.808) | 0.999 | (0.677) | 0.996 | (0.650) |

| | | | | |
|---|---|---|---|---|
| Refinement | | | | |
| Resolution (Å) ¹ | 39.60-1.90 | 33.44-1.75 | 31.89-1.80 | 48.85-1.60 |
| Reflections | 19,843/1,011 | 56,581/2,787 | 52,872/2,725 | 75,315/3,715 |
| (working/test)¹ | | | | |
| R_{factor}/R_{free} (%)^{1, 3} | 18.2/24.2 | 19.0/24.1 | 17.4/22.5 | 18.5/21.6 |
| No. of atoms | 2,261/80/150 | 4,475/122/248 | 4,396/118/306 | 4,499/122/369 |
| (Protein/Ligand/Water) | | | | |
| Model Quality | | | | |
| R.m.s deviations | | | | |
| Bond lengths (Å) | 0.012 | 0.010 | 0.011 | 0.009 |
| Bond angles (°) | 1.121 | 1.068 | 1.153 | 1.049 |
| Average B-factor (Å²) | | | | |
| All Atoms | 27.2 | 37.8 | 36.4 | 28.6 |
| Protein | 26.6 | 37.5 | 35.9 | 27.9 |
| Ligand | 32.5 | 41.6 | 40.1 | 31.6 |
| Water | 32.9 | 41.5 | 41.1 | 35.4 |
| Coordinate | 0.25 | 0.20 | 0.21 | 0.21 |
| error(maximum | | | | |
| likelihood) (Å) | | | | |
| Ramachandran Plot | | | | |
| Most favored (%) | 96.6 | 97.3 | 97.8 | 98.0 |
| Additionally allowed | 3.0 | 2.5 | 2.2 | 2.0 |
| (%) | | | | |

Values in parenthesis are for the highest resolution shell.
R_{merge} = ΣₕₖₗΣᵢ |Iᵢ(hkl) − <I(hkl)>|/ΣₕₖₗΣᵢ Iᵢ(hkl), where Iᵢ(hkl) is the intensity measured for the ith reflection and <I(hkl)> is the average intensity of all reflections with indices hkl.
R_{factor} = Eₕₖₗ ||F_{obs} (hkl) | − |F_{calc} (hkl) ||/Eₕₖₗ |F_{obs} (hkl)|; R_{Free} is calculated in an identical manner using 5% of randomly selected reflections that were not included in the refinement.
Rₘₑₐₛ = redundancy-independent (multiplicity-weighted) R_{merge}. Rₚᵢₘ = precision-indicating (multiplicity-weighted) R_{merge}.
CC_{1/2} is the correlation coefficient of the mean intensities between two random half-sets of data.

### Accession Codes

Coordinates and structure factors for complexes with the following with inhibitors were deposited to the Worldwide Protein Databank (wwPDB) with the accession codes: MERS-CoV 3CLpro complexes: 8c (7T3Y), 9c (7T3Z), 10c (7T40), 14c (7T41) and SARS-CoV-2 3CL^{pro} complexes: 2c (7T42), 3c (7T43), 4c (7T44), 7c (7T45), 8c (7T46), 9c (7T48), 10c (7T49), 11c (7T4A), 14c (7T4B). Atomic coordinates are available upon publication.

### Example 4

### Additional Compounds

Additional compounds have been synthesized and screened against SARS-CoV-2 and MERS-CoV 3C-like protease. Briefly, to a solution of benzyl chloromethyl ether (31.6 mmol; 2 eq) in anhydrous DMF (40 mL) kept at −40° C. was added dropwise a 2M solution of sodium cyclopentadienide in THE (16 mmol; 1 eq) with vigorous stirring for 20 minutes. The reaction mixture was poured into a cold (4° C.) mixture of pentane (120 mL) and water (60 mL). After extraction with cold pentane (2×60 mL) the combined pentane extracts were washed with icy water, dried over anhydrous sodium sulfate and filtered. The pentane was removed by distillation under reduced pressure at 0° C. to yield a cold yellow oily residue of 5-benzyloxymethyl cyclopentadiene which was used in the next step. Using essentially the same procedure as that described in Katsuaki M et al, EPA 0249953A1, Dec. 23, 1987), the acrylic acid ester (2.4 g; 13 mmol) was dissolved in a mixture of DCM/petroleum ether (24 mL of a 7/1 v/v mixture) and the solution was cooled to −15° C. A 1M solution of titanium tetrachloride in petroleum ether (6.5 mL) was added dropwise and stirred for 30 minutes at 15° C. The reaction mixture was cooled to −60° C. and 5-benzyloxymethyl cyclopentadiene (31.9 mmol) was added and the mixture was stirred at −60° C. for 10 h. The temperature was raised to 0° C. and then sodium carbonate decahydrate (13.4 g) was added and the mixture was stirred at room temperature for 0.5 h. The precipitate was filtered off and the filtrate was concentrated. The crude residue was purified by flash chromatography (silica gel/hexane/ethyl ether). The ester was hydrolyzed to the acid by stirring with lithium hydroxide in aqueous THE and the acid was treated with carbonyl diimidazole followed by NaBH₄ or NaBD₄ as described for other synthesis protocols to yield the nondeuterated and deuterated alcohols which were elaborated further to yield the corresponding aldehydes ADR-VI-01 and ADR-VI-02 which were screened against SARS-CoV-2 and MERS-CoV as described in the Examples above. The IC₅₀ values are given in the table below.

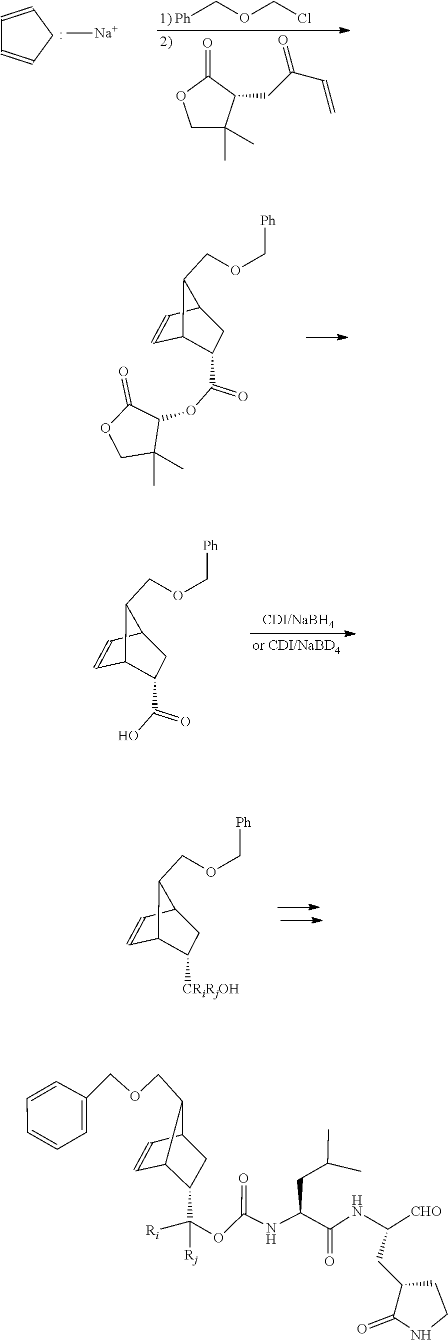

IC₅₀ uM

| | SARS-CoV-2 | MERS-CoV |
|---|---|---|
| | 3CLpro | 3CLpro |
| ADR-VI-01 (Rᵢ = Rⱼ = H) | 0.22 | 0.10 |
| ADR-VI-02 (Rᵢ = Rⱼ = D) | 0.45 | 0.23 |

1. A viral inhibitor compound comprising formula I, or a prodrug, deuterated form, or pharmaceutically-acceptable salt thereof: 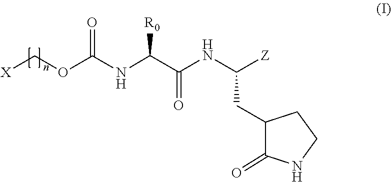
wherein,
n is 0-6;
each R₀ is a branched or unbranched alkyl, cycloalkyl, aryl, arylalkyl, alkenyl, alkynyl, natural or unnatural amino acid side chain, or a combination thereof,
each X is a polycyclic cycloalkane, bridged polycycle, nitrogen-, oxygen-, phosphorous-, or sulfur-containing heterocycle or polycycle, azetidine, or spirocycle; and
each Z is selected from the group consisting of C₁-C₆ hydroxyalkyl, aldehydes, alpha-ketoamides, bisulfite salts, and bisulfite adducts of alpha-ketoamides and alpha-ketoheterocycles.

2. The compound of claim 1, wherein X is a polycyclic cycloalkane, bridged polycycle, nitrogen-containing heterocycle or polycycle, azetidine, or spirocycle.

3. The compound of claim 2, wherein the nitrogen-containing heterocycle comprises at least one ring nitrogen atom and 0-3 additional ring heteroatoms selected from nitrogen, oxygen, phosphorous, and sulfur.

4. The compound of claim 1 or claim 2, wherein X is selected from the group consisting of 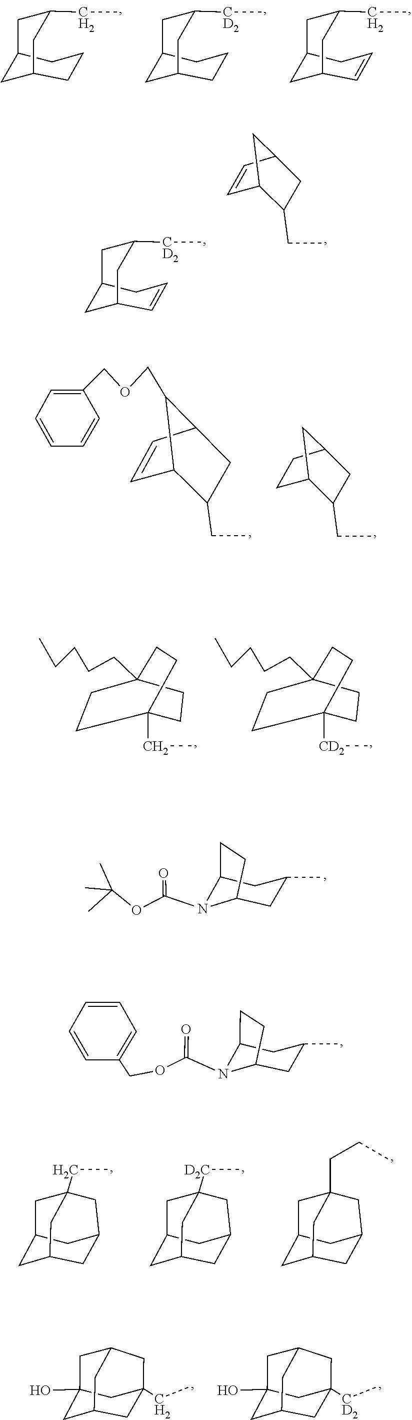 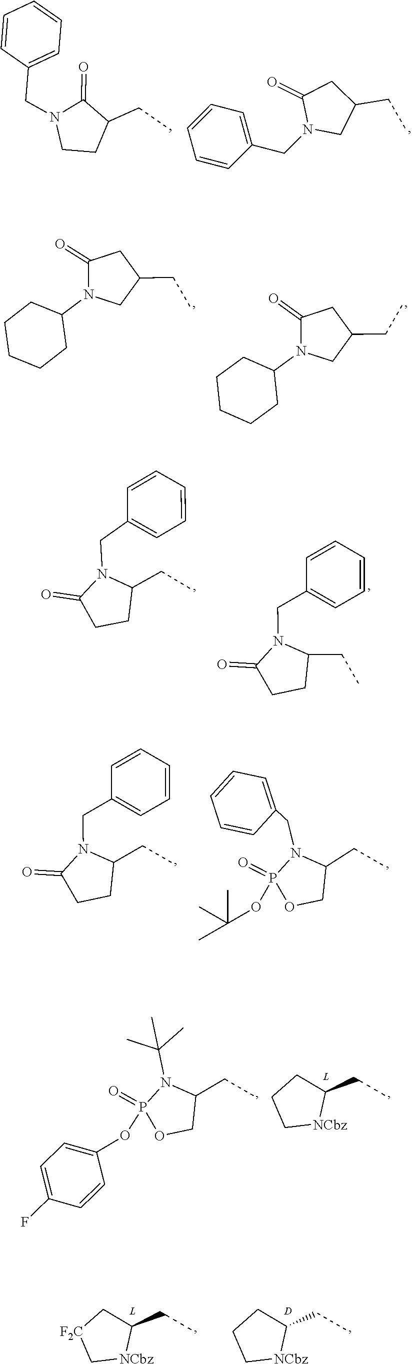 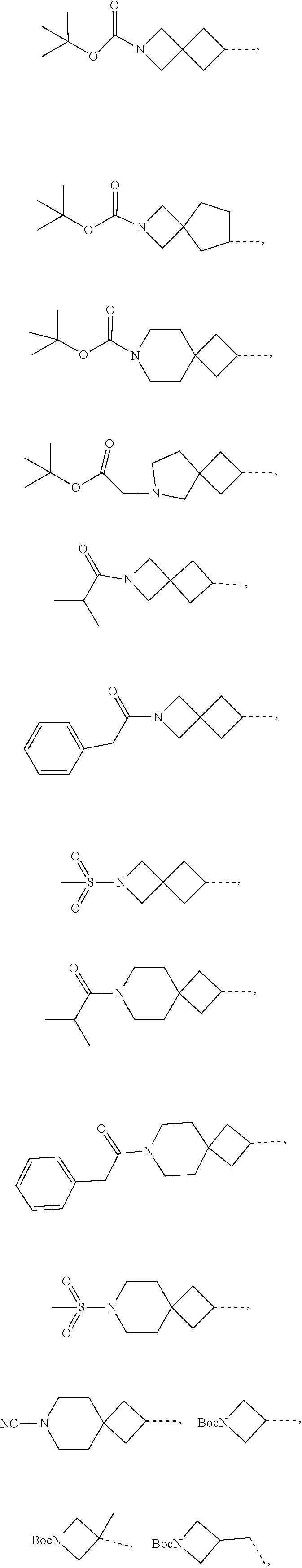
and deuterated forms thereof, prodrugs thereof, or pharmaceutically-acceptable salts thereof.

5. A compound of Table 2, Table 6, Table 7, Table 8, Table 9, Table 10, Table 11, or Table 12, or a pharmaceutically acceptable salt thereof.

6. The compound of claim 5, wherein the compound comprises 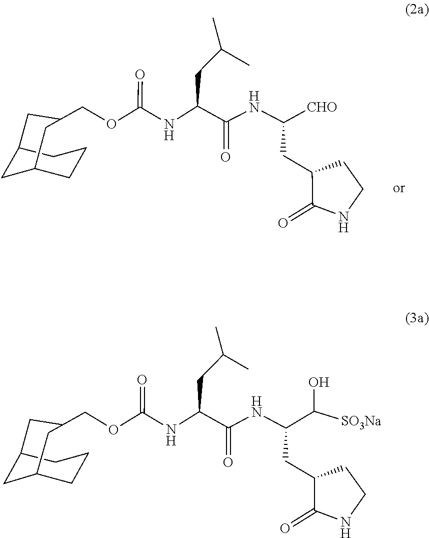
or the deuterated forms thereof, prodrugs thereof, or pharmaceutically-acceptable salts thereof.

7. The compound of any one of claims 1-6, wherein said compound inhibits viral replication of coronavirus as well as against other viruses that belong to the picornavirus-like supercluster, including caliciviruses and picornaviruses.

8. The compound of any one of claims 1-7, wherein said compound inhibits 3C-like protease activity of said virus.

9. The compound of any one of claims 1-8, wherein said compound has broad spectrum activity effective against multiple viruses, including Severe Acute Respiratory Syndrome coronavirus (SARS-CoV), Middle East respiratory syndrome coronavirus (MERS-CoV), and SARS-CoV-2.

10. A method of treating or preventing viral infection from one or more coronaviruses, caliciviruses, or picornaviruses in a subject, said method comprising administering to said subject a therapeutically-effective amount of a first viral inhibitor compound according to any one of claims 1-9.

11. The method of claim 10, wherein said compound is dispersed in a pharmaceutically-acceptable carrier.

12. The method of claim 10 or 11, further comprising providing a unit dosage form of said compound dispersed in said pharmaceutically-acceptable carrier prior to said administering.

13. The method of any one of claims 10 to 12, further comprising administering a second viral inhibitor compound to said subject.

14. The method of claim 13, wherein said second compound is a viral inhibitor compound according to any one of claims 1 to 8, said first compound being different from said second compound.

15. The method of claim 13 or 14, wherein said first and second compounds are co-administered.

16. The method of any one of claims 13 to 15, both of said compounds being dispersed or dissolved in a pharmaceutically-acceptable carrier.

17. The method of any one of claims 10 to 16, wherein said subject is suffering from a viral infection from coronaviruses, caliciviruses, or picornaviruses prior to said administering.

18. The method of any one of claims 10 to 16, wherein said subject is free from a viral infection from coronaviruses, caliciviruses, or picornaviruses prior to said administering.

19. The method of any one of claims 10 to 18, wherein said compound is administered intramuscularly, subcutaneously, intradermally, intranasally, intravenously, orally, or via a transdermal patch.

20. The method of any one of claims 10 to 19, wherein said subject is a human.

21. The method of any one of claims 10 to 19, wherein said subject is a non-human animal.

22. A broad spectrum antiviral composition comprising a first viral inhibitor compound according to any one of claims 1 to 9 dispersed in a pharmaceutically-acceptable carrier.

23. The composition of claim 22, wherein said carrier is selected from the group consisting of sterile isotonic aqueous buffer, normal saline, phosphate buffered saline, DMSO, sterile water, oil-in-water emulsion, water-in-oil emulsion, and mixtures thereof.

24. The composition of claim 22 or 23, said composition comprising from about 5% to about 95% by weight of said viral inhibitor compound, based upon the total weight of said composition taken as 100% by weight.

25. The composition of any one of claims 22 to 24, further comprising a second compound, both of said compounds being dispersed in said pharmaceutically-acceptable carrier.

26. The composition of claim 25, wherein said second compound is a viral inhibitor compound according to any one of claims 1 to 9, said first compound being different from said second compound.

27. The composition of any one of claims 22 to 26, further comprising adjuvants, other active agents, preservatives, buffering agents, salts, and mixtures thereof.

28. A kit comprising: a viral inhibitor compound according to any one of claims 1 to 9; and instructions for administering said compound to a subject in need thereof.

29. The kit of claim 28, wherein said compound is provided in unit dosage form.

30. The kit of claim 28 or 29, wherein said compound is provided in a first container, said kit further comprising a carrier in a second container; and instructions for preparing said antiviral compound for administration to said subject.

31. A method of preventing or inhibiting replication of a virus in a cell, said method comprising contacting said cell with a viral inhibitor compound according to any one of claims 1 to 9, wherein said virus is a coronavirus, calicivirus, or picronavirus.

32. The method of claim 31, wherein said virus is selected from the group consisting of MERS-CoV, SARS-CoV, and SARS-CoV-2.

33. Use of a viral inhibitor compound according to any one of claims 1 to 9 to prepare a therapeutic or prophylactic medicament for the treatment or prevention of a viral infection from coronaviruses, caliciviruses, or picronaviruses in a subject.

34. A method of synthesizing a viral inhibitor compound that inhibits 3C-like protease activity of coronavirus as well as against other viruses that belong to the picornavirus-like supercluster, including caliciviruses and picornaviruses, said method comprising: activating a polycyclic cycloalkane, bridged polycycle, nitrogen-, oxygen-, phosphorous-, or sulfur-containing heterocycle or polycycle, azetidine, or spirocycle compound with a reactive primary or secondary alcohol group with N,N′-disuccinimidyl carbonate (DSC) to yield a mixed carbonate; coupling said mixed carbonate with an amino alcohol precursor of formula I to yield an intermediate alcohol compound; and oxidizing said intermediate alcohol with Dess-Martin periodinane (DMP) to generate the corresponding aldehyde of said intermediate alcohol, wherein said aldehyde is then optionally converted to a ketoamide, bisulfite salt, bisulfite adduct of an alpha-ketoamide, or bisulfite adduct of an alpha-ketoheterocycle.
